(19) United States
(12) Patent Application Publication
(10) Pub. No.: US 2016/0303138 A1
(43) Pub. Date: Oct. 20, 2016

(54) Combination Therapy Of Substituted Oxazolidinones

(71) Applicant: Bayer Intellectual Property GMBH, MONHEIM (DE)

(72) Inventor: Elisabeth Perzborn, Wuppertal (DE)

(73) Assignee: Bayer Intellectual Property GMBH, MONHEIM (DE)

(21) Appl. No.: 15/190,944

(22) Filed: Jun. 23, 2016

Related U.S. Application Data
(62) Division of application No. 12/513,363, filed on Jan. 8, 2010.

(30) Foreign Application Priority Data
Nov. 2, 2006 (DE) 10 2006 051 625.7

(51) Int. Cl. A61K 31/5377; A61K 31/4365; A61K 31/616

(52) U.S. Cl. CPC A61K 31/5377; A61K 31/616; A61K 31/4365

(57) ABSTRACT

The present invention relates to combinations of A) oxazolidinones of the formula (I) with B) acetylsalicylic acid (aspirin) and C) an ADP receptor antagonist, in particular P₂Y₁₂ purinoreceptor blocker, to a process for producing these combinations and to the use thereof as medicaments, in particular for the prophylaxis and/or treatment of thromboembolic disorders.

The present invention relates to combinations of A) oxazolidinones of the formula (I) with B) acetylsalicylic acid (aspirin) and C) an ADP receptor antagonist, in particular P₂Y₁₂ purinoreceptor blocker, to a process for producing these combinations and to the use thereof as medicaments, in particular for the prophylaxis and/or treatment of thromboembolic disorders.

Oxazolidinones of the formula (I) act in particular as selective inhibitors of coagulation factor Xa (FXa) and as anticoagulants (cf. WO 01/47919). Combinations of FXa inhibitors with platelet aggregation inhibitors, anticoagulants, fibrinolytics, lipid-lowering agents, coronary therapeutic agents and/or vasodilators are described in WO 03/000256.

It has been possible to demonstrate an antithrombotic effect of factor Xa inhibitors in numerous animal models (cf. U. Sinha, P. Ku. J. Malinowski, B. Yan Zhu, R M. Scarborough, C K. Marlowe, P W. Wong, P. Hua Lin, S J. Hollenbach. Antithrombotic and hemostatic capacity of factor Xa versus thrombin inhibitors in models of venous and arteriovenous thrombosis. European Journal of Pharmacology 2000, 395, 51-59; A. Betz, Recent advances in Factor Xa inhibitors. Expert Opin. Ther. Patents 2001, 11, 1007; K. Tsong Tan, A. Makin, G. Y H Lip, Factor X inhibitors, Exp. Opin. Investig. Drugs 2003, 12, 799; J. Ruef, H A. Kalus, New antithrombotic drugs on the horizon, Expert Opin. Investig. Drugs 2003, 12, 781; M M. Samama. Synthetic direct and indirect factor Xa inhibitors, Thrombosis Research 2002, 106, V267; M L. Quan, J M. Smallheer, The race to an orally active Factor Xa inhibitor, Recent advances, J. Current Opinion in Drug Discovery& Development 2004, 7, 460-469) and in clinical studies on patients (The Ephesus Study, blood 2000, Vol 96, 490a; The Penthifra Study, blood 2000, Vol 96, 490a; The Pentamaks Study, blood 2000, Vol 96, 490a-491a; The Pentathlon 2000 Study, blood 2000, Vol 96, 491a). Factor Xa inhibitors can therefore preferably be employed in medicaments for the prophylaxis and/or treatment of thromboembolic disorders.

Selective FXa inhibitors show a wide therapeutic window. It was possible to show in numerous animal experimental investigations that FXa inhibitors show an antithrombotic effect in thrombosis models without, or with only a slight, prolonging effect on bleeding times (cf. R. J. Leadly, Coagulationfactor Xa inhibition: biological background and rationale. Curr Top Med Chem 2001; 1, 151-159).

Rivaroxaban (BAY 59-7939) is a novel type of direct factor Xa (FXa) inhibitor which is undergoing clinical development and is to be employed for the treatment and prevention of thromboembolic disorders. The antithrombotic effect of rivaroxaban was shown in animal experimental investigations (Perz orxn, E, Srassburger J, Wilmen A, Pohlmann J, Roehrig S, Schlemmer K H, Straub A, In vitro and in vivo studies of the novel antithrombotic agent BAY 59-7939—an oral, direct Factor Xa inhibitor. JTH 2005; 3: 514-521) and in clinical studies (Kubitza D. Haas S, Novel factor Xa inhibitors for prevention and treatment of thromboembolic diseases. Expert Opin Investig Drugs 2006; 15: 843-855).

It was possible to prove in clinical studies that therapy with inhibitors of platelet aggregation such as aspirin (acetylsalicylic acid) (Antithrombotic Trialists Collaboration; Collaborative meta-analysis of randomized trials of antiplatelet therapy for prevention of death, myocardial infarction. and stroke in high risk patients. BMJ 2002; 324: 71-86), clopidogrel (CAPRIE Steering Committee; A randomised, blinded, trial of clopidogrel versus aspirin in patients at risk of ischemic events (CAPRIE). Lancet 1996; 348: 1329-39) and in particular combination thereof (Peters R J, Mehta S R, Fox K A et al. Effects of aspirin dose when used alone or in combination with clopidogrel in patients with acute coronary syndromes: observations from the Clopidogrel in Unstable angina to prevent Recurrent Events (Cure) study. Circulation 2003; 108: 1682-7; Chen Z M, Jiang L X, Chen Y P et al. Addition of clopidogrel to aspirin in 45,852 patients with acute myocardial infarction: randomised placebo-controlled trial. Lancet 2005; 0.366: 1607-21) leads to a reduction in ischemic events (such as myocardial infarction, stroke). However, despite the evident improvement, therapy with platelet aggregation inhibitors has a limited effect.

It has now surprisingly been found that combinations of oxazolidinones of the formula (I) with acetylsalicylic acid and ADP receptor antagonists, especially P₂Y₁₂ purinoreceptor blockers, have interesting properties and are more suitable for prophylaxis and/or treatment of thromboembolic disorders than are the individual active ingredients alone, combination of oxazolidinones of the formula (I) with acetylsalicylic acid, combination of oxazolidinones of the formula (I) with an ADP receptor antagonist or combination of acetylsalicylic acid and ADP receptor antagonists.

The invention therefore relates to combinations of

A) oxazolidinones of the formula (I) with

B) acetylsalicylic acid and

C) an ADP receptor antagonist.

“Combinations” mean for the purposes of the invention not only dosage forms which comprise all the components (so-called fixed combinations), and combination packs which comprise the components separate from one another, but also components administered simultaneously or sequentially as long as they are employed for the prophylaxis and/or treatment of the same disease. Suitable oxazolidinones of the combination of the invention include, for example, compounds of the formula (I)

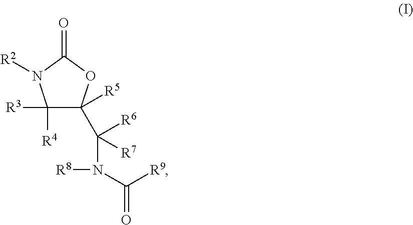

in which:
R¹ is optionally benzo-fused thiophene (thienyl) which may optionally be substituted one or more times;
R² is any organic radical;

R³, R⁴, R⁵, R⁶, R⁷ and R⁸ are identical or different and are hydrogen or (C₁-C₆)-alkyl,

and the pharmaceutically acceptable salts, hydrates and prodrugs thereof.

Preference is given in this connection to compounds of the formula (I) in which
R¹ is optionally benzo-fused thiophene (thienyl) which may optionally be substituted one or more times by a radical from the group of halogen; cyano; nitro; amino; aminomethyl; (C₁-C₈)-alkyl which may in turn be optionally substituted one or more times by halogen; (C₃-C₇)-cycloalkyl; (C₁-C₈)-alkoxy; imidazolinyl; —C(═NH)NH₂; carbamoyl; and mono- and di-(C₁-C₄)-alkylaminocarbonyl,
R² is one of the following groups:
   A-,
   A-M-,
   D-M-A-,
   B-M-A-,
   B-,
   B-M-,
   B-M-B-,
   D-M-B-,
   where:
   the radical “A” is (C₆-C₁₄)-aryl, preferably (C₆-C₁₀)-aryl, in particular phenyl or naphthyl, very particularly preferably phenyl;
   the radical “B” is a 5- or 6-membered aromatic heterocycle which comprises up to 3 heteroatoms and/or hetero chain members, in particular up to 2 heteroatoms and/or hetero chain members, from the series S, N, NO (N-oxide) and O;
   the radical “D” is a saturated or partially unsaturated, mono- or bicyclic, optionally benzo-fused 4- to 9-membered heterocycle which comprises up to three heteroatoms and/or hetero chain members from the series S, SO, SO₂, N, NO (N-oxide) and O;
   the radical “M” is —NH—, —CH₂—, —CH₂CH₂—, —O—, —NH—CH₂—, —CH₂—NH—, —OCH₂—, —CH₂O—, —CONH—, —NHCO—, —COO—, —OOC—, —S—, —SO₂— or a covalent bond;
   where
   the groups “A”, “B” and “D” defined above may in each case optionally be substituted one or more times by a radical from the group of halogen; trifluoromethyl; oxo; cyano; nitro; carbamoyl; pyridyl; (C₁-C₆)-alkanoyl; (C₃-C₇)-cycloalkanoyl; (C₆-C₁₄)-arylcarbonyl; (C₅-C₁₀)-heteroarylcarbonyl; (C₁-C₆)-alkanoyloxymethyloxy; (C₁-C₄)-hydroxyalkylcarbonyl; —COOR²⁷; —SO₂R²⁷; —C(NR²⁷R²⁸)═NR²⁹; —CONR²⁸R²⁹; —SO₂NR²⁸R²⁹; —OR³⁰; —N³⁰R³¹, (C₁-C₆)-alkyl and (C₃-C₇)-cycloalkyl,
   where (C₁-C₆)-alkyl and (C₃-C₇)-cycloalkyl in turn may optionally be substituted by a radical from the group of cyano; —OR²⁷; —NR²⁸R²⁹; —CO(NH)ᵥ(NR²⁷R²⁸) and —C(NR²⁷R²⁸)═NR²⁹,
   where:
   v is either 0 or 1 and
   R²⁷, R²⁸ and R²⁹ are identical or different and are, independently of one another, hydrogen, (C₁-C₄)-alkyl, (C₃-C₇)-cycloalkyl, (C₁-C₄)-alkanoyl, carbamoyl, trifluoromethyl, phenyl or pyridyl,
   and/or
   R²⁷ and R²⁸, or R²⁷ and R²⁹, form together with the nitrogen atom to which they are bonded a saturated or partially unsaturated 5- to 7-membered heterocycle having up to three, preferably up to two, identical or different heteroatoms from the group of N, O and S, and
   R³⁰ and R³¹ are identical or different and are, independently of one another, hydrogen, (C₁-C₄)-alkyl, (C₃-C₇)-cycloalkyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, —CH₂C(NR²⁷R²⁸)═NR²⁹ or —COR³³,
      where
      R³³ is (C₁-C₆)-alkoxy, (C₁-C₄)-alkoxy-(C₁-C₄)-alkyl, (C₁-C₄)-alkoxycarbonyl-(C₁-C₄)-alkyl, (C₁-C₄)-aminoalkyl, (C₁-C₄)-alkoxycarbonyl, (C₁-C₄)-alkanoyl-(C₁-C₄)-alkyl, (C₃-C₇)-cycloalkyl, (C₂-C₆)-alkenyl, (C₁-C₈)-alkyl which may optionally be substituted by phenyl or acetyl, or is (C₆-C₁₄)-aryl, (C₅-C₁₀)-heteroaryl, trifluoromethyl, tetrahydrofuranyl or butyrolactone,
R³, R⁴, R⁵, R⁶, R⁷ and R⁸ are identical or different and are hydrogen or (C₁-C₆)-alkyl,

and the pharmaceutically acceptable salts, hydrates and prodrugs thereof.

Preference is likewise given in this connection to compounds of the general formula (I)

in which
R¹ is thiophene (thienyl), in particular 2-thiophene, which may optionally be substituted one or more times by halogen, preferably chlorine or bromine, amino, aminomethyl or (C₁-C₈)-alkyl, preferably methyl, where the (C₁-C₈)-alkyl radical may optionally in turn be substituted one or more times by halogen, preferably fluorine,
R² is one of the following groups:
   A-,
   A-M-,
   D-M-A-,
   B-M-A-,
   B-,
   B-M-,
   B-M-B-,
   D-M-B-,
   where:
   the radical “A” is (C₆-C₁₄)-aryl, preferably (C₆-C₁₀)-aryl, in particular phenyl or naphthyl, very particularly preferably phenyl;
   the radical “B” is a 5- or 6-membered aromatic heterocycle which comprises up to 3 heteroatoms and/or hetero chain members, in particular up to 2 heteroatoms and/or hetero chain members, from the series S, N, NO (N-oxide) and O;
   the radical “D” is a saturated or partially unsaturated 4- to 7-membered heterocycle which comprises up to three heteroatoms and/or hetero chain members from the series S, SO, SO₂, N, NO (N-oxide) and O;
   the radical “M” is —NH—, —CH₂—, —CH₂CH₂—, —O—, —NH—CH₂—, —CH₂—NH—, —OCH₂—, —CH₂O—, —CONH—, —NHCO—, —COO—, —OOC—, —S— or a covalent bond;
   where
   the groups “A”, “B” and “D” defined above may in each case optionally be substituted one or more times by a radical from the group of halogen; trifluoromethyl; oxo; cyano; nitro; carbamoyl; pyridyl; (C₁-C₆)-alkanoyl; (C₃-C₇)-cycloalkanoyl; (C₆-C₁₄)-arylcarbonyl; (C₅-C₁₀)-heteroarylcarbonyl; (C₁-C₆)-alkanoyloxymethyloxy; —COOR²⁷; —SO₂R²⁷; —C(NR²⁷R²⁸)═NR²⁹; —CONR²⁸R²⁹; —SO₂NR²⁸R²⁹; —OR³⁰; —NR³⁰R³³, (C₁-C₆)-alkyl and (C₃-C₇)-cycloalkyl,
   where (C₁-C₆)-alkyl and (C₃-C₇)-cycloalkyl may in turn optionally be substituted by a radical from the group of cyano; —OR²⁷; —NR²⁸R²⁹; —CO(NH)ᵥ(NR²⁷R²⁸) and —C(NR²⁷R²⁸═NR²⁹,
   where:
   v is either 0 or 1, and
   R²⁷, R²⁸ and R²⁹ are identical or different and are, independently of one another, hydrogen, (C₁-C₄)-alkyl or (C₃-C₇)-cycloalkyl,
   and/or
   R²⁷ and R²⁸, or R²⁷ and R²⁹, form together with the nitrogen atom to which they are bonded a saturated or partially unsaturated 5- to 7-membered heterocycle having up to three, preferably up to two, identical or different heteroatoms from the group of N, O and S, and
   R³⁰ and R³¹ are identical or different and are, independently of one another, hydrogen, (C₁-C₄)-alkyl, (C₃-C₇)-cycloalkyl, (C₁-C₄-alkylsulfonyl, (C₁-C₄)-hydroxyalkyl, (C₁-C₄-aminoalkyl, di-(C₁-C₄)-alkylamino-(C₁-C₄)-alkyl, (C₁-C₄)-alkanoyl, (C₆-C₁₄)-arylcarbonyl, (C₅-C₁₀)-heteroarylcarbonyl, (C₁-C₄)-alkylaminocarbonyl or —CH₂C(NR²⁷R²⁸)═NR²⁹,

R³, R⁴, R⁵, R⁶, R⁷ and R⁸ are identical or different and are hydrogen or (C₁-C₆)-alkyl,

and the pharmaceutically acceptable salts, hydrates and prodrugs thereof.

Particular preference is given in this connection to compounds of the general formula (I)

in which
R¹ is thiophene (thienyl), in particular 2-thiophene, which may optionally be substituted one or more times by halogen, preferably chlorine or bromine, or (C₁-C₈)-alkyl, preferably methyl, where the (C₁-C₈)-alkyl radical may in turn optionally be substituted one or more times by halogen, preferably fluorine.
R² is one of the following groups:
   A-,
   A-M-,
   D-M-A-,
   B-M-A-,
   B-,
   B-M-,
   B-M-B-,
   D-M-B-,
   where:
   the radical “A” is phenyl or naphthyl, in particular phenyl;
   the radical “B” is a 5- or 6-membered aromatic heterocycle which comprises up to 2 heteroatoms from the series S, N, NO (N-oxide) and O;
   the radical “D” is a saturated or partially unsaturated 5- or 6-membered heterocycle which comprises up to two heteroatoms and/or hetero chain members from the series S, SO, SO₂, N, NO (N-oxide) and O;
   the radical “M” is —NH—, —O—, —NH—CH₂—, —CH₂—NH—, —OCH₂—, —CH₂O—, —CONH—, —NHCO— or a covalent bond;
   where
   the groups “A”, “B” and “D” defined above may in each case optionally be substituted one or more times by a radical from the group of halogen; trifluoromethyl; oxo; cyano; pyridyl; (C₁-C₃)-alkanoyl; (C₆-C₁₀)-arylcarbonyl; (C₅-C₆)-heteroarylcarbonyl; (C₁-C₃)-alkanoyloxymethyloxy; —C(NR²⁷R²⁸)═NR²⁹; —CONR²⁸R²⁹; —SO₂NR²⁸R²⁹; —OH; —NR³⁰R³¹; (C₁-C₄)-alkyl; and cyclopropyl, cyclopentyl or cyclohexyl,
   where (C₁-C₄)-alkyl and cyclopropyl, cyclopentyl or cyclohexyl may in turn optionally be substituted by a radical from the group of cyano; —OH; —OCH₃; —NR²⁸R²⁹; —CO(NH)ᵥ(NR²⁷R²⁸) and —C(NR²⁷R²⁸)═NR²⁹,
   where:
   v is either 0 or 1, preferably 0, and
   R²⁷, R²⁸ and R²⁹ are identical or different and are, independently of one another, hydrogen, (C₁-C₄)-alkyl or else cyclopropyl, cyclopentyl or cyclohexyl, and/or
   R²⁷ and R²⁸, or R²⁷ and R²⁹, may form together with the nitrogen atom to which they are bonded a saturated or partially unsaturated 5- to 7-membered heterocycle having up to two identical or different heteroatoms from the group of N, O and S, and
   R³⁰ and R³¹ are identical or different and are, independently of one another, hydrogen, (C₁-C₄)-alkyl, cyclopropyl, cyclopentyl, cyclohexyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₃)-alkanoyl or phenylcarbonyl,
R³, R⁴, R⁵, R⁶, R⁷ and R⁸ are identical or different and are hydrogen or (C₁-C₆)-alkyl,

and the pharmaceutically acceptable salts, hydrates and prodrugs thereof.

Especial preference is given in this connection to compounds of the general formula (I)

in which
R¹ is 2-thiophene which may optionally be substituted in position 5 by a radical from the group chlorine, bromine, methyl or trifluoromethyl,
R² is one of the following groups:
   A-,
   A-M-,
   D-M-A-,
   B-M-A-,
   B-,
   B-M-,
   B-M-B-,
   D-M-B-,
   where:
   the radical “A” is phenyl or naphthyl, in particular phenyl:
   the radical “B” is a 5- or 6-membered aromatic heterocycle which comprises up to 2 heteroatoms from the series S, N, NO (N-oxide) and O;
   the radical “D” is a saturated or partially unsaturated 5- or 6-membered heterocycle which comprises a nitrogen atom and optionally a further heteroatom and/or hetero chain member from the series S, SO, SO₂ and O; or up to two heteroatoms and/or hetero chain members
   from the series S, SO, SO₂ and O;
   the radical “M” is —NH₂, —O—, —NH—CH₂—, —CH₂—NH—, —OCH₂—, —CH₂O—, —CONH—, —NHCO— or a covalent bond;
   where
   the groups “A”, “B” and “D” defined above may in each case optionally be substituted one or more times by a radical from the group of halogen; trifluoromethyl; oxo; cyano; pyridyl; (C₁-C₃)-alkanoyl; (C₆-C₁₀)-arylcarbonyl; (C₅-C₆)-heteroarylcarbonyl; (C₁-C₃)-alkanoyloxymethyloxy; —CONR²⁸R²⁹; —SO₂NR²⁸R²⁹; —OH; —NR³⁰R³¹; (C₁-C₄)-alkyl; and cyclopropyl, cyclopentyl or cyclohexyl,
   where (C₃-C₄-alkyl and cyclopropyl, cyclopentyl or cyclohexyl may in turn optionally be substituted by a radical from the group of cyano; —OH; —OCH₃; —NR²⁸R²⁹; —CO(NH), (NR²⁷R²⁸) and —C(NR²⁷R²⁸)═NR²⁹,
   where:
   v is either 0 or 1, preferably 0, and
   R²⁷, R²⁸ and R²⁹ are identical or different and are, independently of one another, hydrogen, (C₁-C₄)-alkyl or else cyclopropyl, cyclopentyl or cyclohexyl, and/or
   R²⁷ and R²⁸, or R²⁷ and R²⁹, may form together with the nitrogen atom to which they are bonded a saturated or partially unsaturated 5- to 7-membered heterocycle having up to two identical or different heteroatoms from the group of N, O and S, and
   R³⁰ and R³¹ are identical or different and are, independently of one another, hydrogen, (C₁-C₄)-alkyl, cyclopropyl, cyclopentyl, cyclohexyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₃)-alkanoyl or phenylcarbonyl,
   R³, R⁴, R⁵, R⁶, R⁷ and R⁸ are identical or different and are hydrogen or (C₁-C₄)-alkyl,

and the pharmaceutically acceptable salts, hydrates and prodrugs thereof.

Very particular preference is given in this connection to compounds of the general formula (I)

in which
R¹ is 2-thiophene which is substituted in position 5 by a radical from the group of chlorine, bromine, methyl or trifluoromethyl.
R² is D-A-:
   where:
   the radical “A” is phenylene:
   the radical “D” is a saturated 5- or 6-membered heterocycle
   which is linked via a nitrogen atom to “A”,
   which has a carbonyl group in direct vicinity to the linking nitrogen atom, and
   in which a ring carbon member may be replaced by a heteroatom from the series S, N and O;
   where
   the group “A” defined above may optionally be substituted once or twice in the meta position relative to the linkage to the oxazolidinone by a radical from the group of fluorine, chlorine, nitro, amino, trifluoromethyl, methyl or cyano,
R³, R⁴, R⁵, R⁶, R⁷ and R⁸ are hydrogen,

and the pharmaceutically acceptable salts, hydrates and prodrugs thereof.

Very particular preference is likewise given in this connection to the compound having the following formula

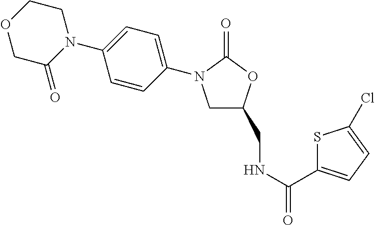 [SMILES: O=C(NC[C@H]1CN(c2ccc(N3CCOCC3=O)cc2)C(=O)O1)c1ccc(Cl)s1]

and the pharmaceutically acceptable salts, hydrates and prodrugs thereof.

To date, oxazolidinones have been described essentially only as antibiotics, and in a few cases also as MAO inhibitors and fibrinogen antagonists (Review: Riedl, B., Endermann, R., Exp. Opin. Ther. Patents 1999, 9 (5), 625), and a small 5-[acylaminomethyl] group (preferably 5-[acetylaminomethyl]) appears to be essential for the antibacterial effect.

Substituted aryl- and heteroarylphenyloxazolidinones in which a monosubstituted or polysubstituted phenyl radical may be bonded to the N atom of the oxazolidinone ring and which may have in position 5 of the oxazolidinone ring an unsubstituted N-methyl-2-thiophenecarboxamide residue, and their use as substances with antibacterial activity are disclosed in the U.S. Pat. No. 5,929,248, U.S. Pat. No. 5,801,246, U.S. Pat. No. 5,756,732, U.S. Pat. No. 5,654,435, U.S. Pat. No. 5,654,428 and U.S. Pat. No. 5,565,571.

In addition, benzamidine-containing oxazolidinones are known as synthetic intermediates in the synthesis of factor Xa inhibitors or fibrinogen antagonists (WO-A-99/31092, EP-A-623615).

The compounds of the formula (I) may, depending on the substitution pattern, exist in stereoisomeric forms which either are related as image and mirror image (enantiomers) or are not related as image and mirror image (diastereomers). Both the enantiomers or diastereomers and respective mixtures thereof are included. The racemic forms can, just like the diastereomers, be separated in a known manner into the stereoisomerically pure constituents.

Certain compounds of the formula (I) may also exist in tautomeric forms. This is known to the skilled worker, and such compounds are likewise included.

Physiologically acceptable, i.e. pharmaceutically acceptable, salts may be salts of the compounds of the invention with inorganic or organic acids. Preferred salts are those with inorganic acids such as, for example, hydrochloric acid, hydrobromic acid, phosphoric acid or sulfuric acid, or salts with organic carboxylic or sulfonic acids such as, for example, acetic acid, trifluoroacetic acid, propionic acid, maleic acid, fumaric acid, malic acid, citric acid, tartaric acid, lactic acid, benzoic acid, or methanesulfonic acid, ethanesulfonic acid, benzenesulfonic acid, toluenesulfonic acid or naphthalenedisulfonic acid.

Pharmaceutically acceptable salts which may also be mentioned are salts with conventional bases, such as, for example, alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylamine, procaine, dibenzylamine, N-methylmorpholine, dihydroabietylamine or methylpiperidine.

“Hydrates” refers to those forms of the compounds of the above formula (I) which form a molecular compound (solvate) in the solid or liquid state through hydration with water. In the hydrates, the water molecules are attached through secondary valencies by intermolecular forces, in particular hydrogen bonds. Solid hydrates contain water as so-called water of crystallization in stoichiometric ratios, and the water molecules do not have to be equivalent in terms of their binding state. Examples of hydrates are sesquihydrates, monohydrates, dihydrates or trihydrates. Equally suitable are also the hydrates of salts of the compounds of the invention.

“Prodrug” refers to those forms of the compounds of the above formula (I) which may themselves be biologically active or inactive but can be converted into the corresponding biologically active form (for example metabolically, solvolytically or in another way).

Halogen is fluorine, chlorine, bromine and iodine. Chlorine or fluorine are preferred.

(C₁-C₈)-Alkyl is a straight-chain or branched alkyl radical having 1 to 8 carbon atoms. Examples which may be mentioned are: methyl, ethyl, n-propyl, isopropyl, n-butyl, isobutyl, tert-butyl, n-pentyl and n-hexyl. The corresponding alkyl groups with fewer carbon atoms are derived analogously from this definition, such as, for example, (C₁-C₆)-alkyl and (C₁-C₄)-alkyl. It is generally true that (C₁-C₄)-alkyl is preferred.

The meaning of the corresponding constituent of other more complex substituents is also derived from this definition, such as, for example, in the case of alkylsulfonyl, hydroxyalkyl, hydroxyalkylcarbonyl, alkoxy-alkyl, alkoxycarbonyl-alkyl, alkanoylalkyl, aminoalkyl or alkylamino-alkyl.

(C₃-C₇)-Cycloalkyl is a cyclic alkyl radical having 3 to 7 carbon atoms. Examples which may be mentioned are: cyclopropyl, cyclobutyl, cyclopentyl, cyclohexyl or cycloheptyl. The corresponding cycloalkyl groups with fewer carbon atoms are derived analogously from this definition, such as, for example, (C₃-C₅)-cycloalkyl. Cyclopropyl, cyclopentyl and cyclohexyl are preferred.

The meaning of the corresponding constituent of other more complex substituent such as, for example, cycloalkanoyl is also derived from this definition.

(C₂-C₆)-Alkenyl is a straight-chain or branched alkenyl radical having 2 to 6 carbon atoms. A straight-chain or branched alkenyl radical having 2 to 4 carbon atoms is preferred. Examples which may be mentioned are: vinyl, allyl, isopropenyl and n-but-2-en-1-yl.

(C₁-C₈)-Alkoxy is a straight-chain or branched alkoxy radical having 1 to 8 carbon atoms. Examples which may be mentioned are: methoxy, ethoxy, n-propoxy, isopropoxy, n-butoxy, isobutoxy, tert-butoxy, n-pentoxy, n-hexoxy, n-heptoxy and n-octoxy. The corresponding alkoxy groups with fewer carbon atoms are derived analogously from this definition, such as, for example, (C₁-C₆)-alkoxy and (C₁-C₄)-alkoxy. It is generally true that (C₁-C₄)-alkoxy is preferred.

The meaning of the corresponding constituent of other more complex substituents such as, for example, alkoxy-alkyl, alkoxycarbonyl-alkyl and alkoxycarbonyl is also derived from this definition.

Mono- or di-(C₁-C₄)-alkylaminocarbonyl is an amino group which is linked via a carbonyl group and which has a straight-chain or branched or two identical or different straight-chain or branched alkyl substituents each having 1 to 4 carbon atoms. Examples which may be mentioned are: methylamino, ethylamino, n-propylamino, isopropylamino, t-butylamino, N,N-dimethylamino, N,N-diethylamino, N-ethyl-N-methylamino, N-methyl-N-n-propylamino, N-isopropyl-N-n-propylamino and N-t-butyl-N-methylamino.

(C₁-C₆)-Alkanoyl is a straight-chain or branched alkyl radical having 1 to 6 carbon atoms which has a double bonded oxygen atom in position 1 and is linked via position 1. Examples which may be mentioned are: formyl, acetyl, propionyl, n-butyryl, i-butyryl, pivaloyl, n-hexanoyl. The corresponding alkanoyl groups with fewer carbon atoms are derived analogously from this definition, such as, for example, (C₁-C₅)-alkanoyl, (C₁-C₄)-alkanoyl and (C₁-C₃)-alkanoyl. It is generally true that (C₁-C₃)-alkanoyl is preferred.

The meaning of the corresponding constituent of other more complex substituents such as, for example, cycloalkanoyl and alkanoylalkyl is also derived from this definition.

(C₃-C₇)-Cycloalkanoyl is a cycloalkyl radical as defined above which has 3 to 7 carbon atoms and which is linked via a carbonyl group.

(C₁-C₆)-Alkanoyloxymethloxy is a straight-chain or branched alkanoyloxymethyloxy radical having 1 to 6 carbon atoms. Examples which may be mentioned are: acetoxymethyloxy, propionoxymethyloxy, n-butyroxymethyloxy, i-butyroxymethyloxy, pivaloyloxymethyloxy, n-hexanoyloxymethyloxy. The corresponding alkanoyloxymethyloxy groups with fewer carbon atoms, such as, for example, (C₁-C₃)-alkanoyloxymethyloxy, are derived analogously from this definition. It is generally true that (C₁-C₃)-alkanoyloxymethyloxy is preferred.

(C₆-C₁₄)-Aryl is an aromatic radical having 6 to 14 carbon atoms. Examples which may be mentioned are: phenyl, naphthyl, phenanthrenyl and anthracenyl. The corresponding aryl groups with fewer carbon atoms, such as, for example, (C₆-C₁₀)-aryl, are derived analogously from this definition. It is generally true that (C₆-C₁₀)-aryl is preferred.

The meaning of the corresponding constituent of other more complex substituents such as, for example, arylcarbonyl is also derived from this definition.

(C₅-C₁₀)-Heteroaryl or a 5- to 10-membered aromatic heterocycle having up to 3 heteroatoms and/or hetero chain members from the series S, O, N and/or NO (N-oxide) is a mono- or bicyclic heteroaromatic system which is linked via a ring carbon atom of the heteroaromatic system, optionally also via a ring nitrogen atom of the heteroaromatic system. Examples which may be mentioned are: pyridyl, pyridyl N-oxide, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl. The corresponding heterocycles with a smaller ring size such as, for example, 5- or 6-membered aromatic heterocycles are derived analogously from this definition. It is generally true that 5- or 6-membered aromatic heterocycles such as, for example, pyridyl, pyridyl N-oxide, pyrimidyl, pyridazinyl, furyl and thienyl are preferred.

The meaning of the corresponding constituent of other more complex substituent such as, for example, (C₅-C₁₀)-heteroarylcarbonyl is also derived from this definition.

A 3- to 9-membered saturated or partially unsaturated, mono- or bicyclic, optionally benzo-fused heterocycle having up to 3 heteroatoms and/or hetero chain members from the series S, SO, SO₂, N, NO (N-oxide) and/or O is a heterocycle which may comprise on or more double bonds, which may be mono- or bicyclic, in which a benzene ring may be fused to two adjacent ring carbon atoms, and which is linked via a ring carbon atom or a ring nitrogen atom. Examples which may be mentioned are: tetrahydrofuryl, pyrrolidinyl, pyrrolinyl, piperidinyl, 1,2-dihydropyridinyl, 1,4-dihydropyridinyl, piperazinyl, morpholinyl, morpholinyl N-oxide, thiomorpholinyl, azepinyl, 1,4-diazepinyl and cyclohexyl, Piperidinyl, morpholinyl and pyrrolidinyl are preferred.

The corresponding cyclic systems with a smaller ring size, such as, for example, 5- to 7-membered cyclic systems, are derived analogously from this definition.

The compounds of the formula (I) can be prepared as described in WO 01/47919.

A preferred compound A) of the formula (I) for use in combinations is 5-chloro-N-({5S)-2-oxo-3-[4-(3-oxo-4-morpholinyl)phenyl]-1,3-oxazolidin-5-yl}methyl)-2-thiophenecarboxamide (rivaroxaban), the compound of Example 44.

A preferred compound C) of an ADP receptor antagonist is a P₂Y₁₂ purinoreceptor blocker.

A preferred compound C) of an ADP receptor antagonist, in particular of a P₂Y₁₂ receptor blocker, is a thienopyridine such as, for example, clopidogrel (Plavix) or prasugrel, or an adenine nucleotide analog such as, for example, cangrelor.

A particularly preferred compound C) of an ADP receptor antagonist, in particular of a P₂Y₁₂ receptor blocker, is clopidogrel (Plavix).

The invention therefore preferably relates to combinations of

A) an oxazolidinone of the formula (I) with

B) acetylsalicylic acid and

C) clopidogrel, prasugrel or cangrelor.

The invention therefore preferably also relates to combinations of

A) an oxazolidinone of the formula (I) with

B) acetylsalicylic acid and

C) clopidogrel.

The invention therefore very particularly preferably relates to the combination of
A) 5-chloro-N-({(5S)-2-oxo-3-[4-(3-oxo-4-morpholinyl)phenyl]-1,3-oxazolidin-5-yl}methyl)-2-thiophenecarboxamide (rivaroxaban) of the formula

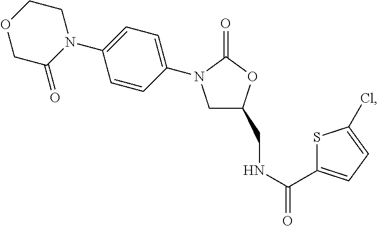 [SMILES: O=C(NC[C@H]1CN(c2ccc(N3CCOCC3=O)cc2)C(=O)O1)c1ccc(Cl)s1]

with
B) acetylsalicylic acid and
C) clopidogrel.

A low-dose FXa inhibitor such as rivaroxaban (Example 44) combined with acetylsalicylic acid and an ADP receptor antagonist such as clopidogrel leads to a potent synergistic antithrombotic effect and is superior to the effect of the combination of oxazolidinones of the formula (F) with acetylsalicylic acid or the combination of oxazolidinones of the formula (I) with an ADP receptor antagonist, and the combination of acetylsalicylic acid and an ADP receptor antagonist alone. Rivaroxaban, given in dosages which show no antithrombotic effect when used alone, leads in combination with acetylsalicylic acid and clopidogrel, a P₂Y₁₂ receptor blocker (ADP receptor antagonist), to a considerable increase in the potency of the antithrombotic effect of the platelet aggregation inhibitors in a thrombosis model.

The combinations of the invention are particularly suitable for the treatment and/or prophylaxis of thromboembolic disorders.

“Thromboembolic disorders” include in the context of the present invention in particular disorders such as myocardial infarction with ST segment elevation (STEMI) and without ST segment elevation (non-STEMI), stable angina pectoris, unstable angina pectoris, reocclusions and restenoses following coronary interventions such as angioplasty or aortocoronary bypass, peripheral arterial occlusive diseases, pulmonary embolisms, deep vein thromboses and renal vein thromboses, transient ischemic attacks, and thrombotic and thromboembolic stroke.

The combinations of the invention are therefore also suitable for the prevention and treatment of cardiogenic thromboembolisms such as, for example, cerebral ischemias, stroke and systemic thromboembolisms and ischemias, in patients with acute, intermittent or persistant cardiac arrhythmias, such as, for example, atrial fibrillation, and those undergoing cardioversion, and for the prevention and treatment of cardiogenic thromboembolisms such as, for example, cerebral ischemias, stroke and systemic thromboembolisms and ischemias in patients with heart valve disorders or with artificial heart valves.

The combinations of the invention are additionally suitable for the treatment of disseminated intravascular coagulation (DIC).

Thromboembolic complications also occur in association with microangiopathic hemolytic anemias, extracorporeal circulations, such as hemodialysis, and heart valve prostheses.

The combinations of the invention are additionally suitable also for the prophylaxis and/or treatment of atherosclerotic vascular disorders and inflammatory disorders such as rheumatic disorders of the muscular skeletal system, furthermore likewise for the prophylaxis and/or treatment of Alzheimer's disease.

The combinations of the invention can additionally be employed for inhibiting tumor growth and metastasis formation, for microangiopathies, age-related macular degeneration, diabetic retinopathy, diabetic nephropathy and other microvascular disorders, and for the prevention and treatment of thromboembolic complications such as, for example, venous thromboembolism in tumor patients, especially those undergoing major surgical procedures or chemotherapy or radiotherapy.

The individual active ingredients of the combinations are disclosed in the literature and, for the most part, commercially available. They can where appropriate, just like oxazolidinones of the formula (I), be employed in subtherapeutically effective doses.

All usual administration forms are suitable for administering the combinations of the invention. Administration preferably takes place orally, lingually, sublingually, buccally, rectally, topically or parenterally (i.e. avoiding the intestinal tract, i.e. intravenous, intraarterial, intracardiac, intracutaneous, subcutaneous, transdermal, intraperitoneal or intramuscular).

The present invention includes pharmaceutical preparations which, besides non-toxic, inert pharmaceutically suitable excipients and/or carriers, comprise one or more combinations of the invention or which consist of a combination of the invention, and processes for producing these preparations.

The combinations of the invention are intended to be present in the abovementioned pharmaceutical preparations in a concentration of about 0.1 to 99.5, preferably about 0.5 to 95, % by weight of the complete mixture.

The abovementioned pharmaceutical preparations may, besides the combinations of the invention, also comprise further active pharmaceutical ingredients.

The abovementioned pharmaceutical preparations can be produced in a conventional way by known methods, e.g. by mixing the active ingredient or active ingredients with the carrier(s).

It has generally proved advantageous to administer the combinations of the invention in total amounts of from about 0.001 to 100 mg/kg, preferably about 0.01 to 100 mg/kg, in particular about 0.1 to 10 mg/kg, of body weight every 24 hours, where appropriate in the form of a plurality of single doses, to achieve the desired results.

It may nevertheless be necessary where appropriate to depart from the aforementioned amounts, in particular depending on the body weight, on the nature of the administration route, the type and severity of the disorder, on the individual behavior toward the medicament, on the nature of the formulation and on the time or interval over which administration takes place. Thus, it may be sufficient in some cases to make do with less than the aforementioned minimum amount, whereas in other cases the upper limit mentioned must be exceeded. It may be advisable, for example when relatively large amounts are administered, to distribute these over the day, in particular either in a plurality of single doses or as continuous infusion.

The invention therefore further relates to the combinations defined above for the prophylaxis and/or treatment of disorders.

The invention further relates to medicaments comprising at least one of the combinations defined above and, where appropriate, further active pharmaceutical ingredients.

The invention further relates to the use of the combinations defined above for producing medicaments for the prophylaxis and/or treatment of the disorders described above, preferably thromboembolic disorders, in particular myocardial infarction with ST segment elevation (STEMI) and without ST segment elevation (non-STEMI), stable angina pectoris, unstable angina pectoris, reocclusions and restenoses following coronary interventions such as angioplasty or aortocoronary bypass, peripheral arterial occlusive diseases, pulmonary embolisms, deep vein thromboses and renal vein thromboses, transient ischemic attacks, and thrombotic and thromboembolic stroke.

The percentage data in the following examples are based in each case on weight; parts are pats by weight.

### EXAMPLES

### A Assessment of the Physiological Activity

1. Physiological Activity of Compounds of the Formula (I)

The compounds of the formula (I) act in particular as selective inhibitors of coagulation factor Xa and do not inhibit, or also inhibit only at distinctly higher concentrations, other serine proteases such as thrombin, plasmin or trypsin.

Inhibitors of coagulation factor Xa are referred to as “selective” when their IC₅₀ values for factor Xa inhibition are 100-fold, preferably 500-fold, in particular 1000-fold, smaller than the IC₅₀ values for the inhibition of other serine proteases, in particular thrombin, plasmin and trypsin, reference being made concerning the test methods for the selectivity to the test methods of Examples A-1) a.1) and a.2) described below.

The particularly advantageous biological properties of the compounds of the formula (f) can be ascertained by the following methods.

a) Test Description (In Vitro)

a.1) Measurement of Factor Xa Inhibition

The enzymatic activity of human factor Xa (FXa) was measured via the conversion of an FXa-specific chromogenic substrate. In this case, factor Xa eliminates p-nitroaniline from the chromogenic substrate. The determinations were carried out in microtiter plates as follows.

The test substances were dissolved in various concentrations in DMSO and incubated with human FXa (0.5 nmol/l dissolved in 50 mmol/l tris buffer [C,C,C-tris(hydroxymethyl)aminomethane], 150 nmol/l NaCl, 0.1% BSA (bovine serum albumine), pH=8.3) at 25° C. for 10 minutes. Pure DMSO serves as control. The chromogenic substrate (150 μmol/l Pefachrome® FXa from Pentapharm) was then added. After incubation at 25° C. for 20 minutes, the extinction at 405 nm was determined. The extinctions of the test mixtures with test substance were compared with the control mixtures without test substance, and the IC₅₀ values were calculated therefrom.

a.2) Selectivity Determination

Selective FXa inhibition was demonstrated by investigating the inhibition by the test substances of other human serine proteases such as thrombin, trypsin, plasmin. The enzymatic activity of thrombin (75 mU/ml), trypsin (500 mU/ml) and plasmin (3.2 nmol/l) was determined by dissolving these enzymes in tris buffer (100 nmol/l, 20 mmol/l CaCl₂, pH=8.0) and incubating with test substance or solvent for 10 minutes. The enzymatic reaction was then started by adding the appropriate specific chromogenic substrates (Chromozym Thrombin® from Boehringer Mannheim, Chromozym Trypsin® from Boehringer Mannheim, Chromozym Plasmin® from Boehringer Mannheim), and the extinction was determined at 405 nm after 20 minutes. All determinations were carried out at 37° C. The extinctions of the test mixtures with test substance were compared with the control samples without test substance, and the IC₅₀ values were calculated therefrom.

a.3) Determination of the Anticoagulant Effect

The anticoagulant effect of the test substances was determined in vitro in human plasma. For this purpose, human blood was collected in a 0.11 molar sodium citrate solution in the sodium citrate/blood mixing ratio of 1/9. The blood was thoroughly mixed after collection and centrifuged at about 2000 g for 10 minutes. The supernatant was removed by pipette. The prothrombin time (PT, synonym: Quick's test) was determined in the presence of varying concentrations of test substance or the appropriate solvent using a commercially available test kit (Neoplastin® from Boehringer Mannheim). The test compounds were incubated with the plasma at 37° C. for 10 minutes. Coagulation was then induced by adding thromboplastin, and the time of onset of coagulation was determined. The concentration of test substance which brings about a doubling of the prothrombin time was found.

2. Physiological Activity of the Combinations of Compounds of the Formula (I)

a) Determination of the Antithrombotic Effect in an Arteriovenous Shunt Model in Rats

Fasting male rats (strain: HSD CPB:WU) were anesthetized by intraperitoneal administration of a Rompun/Ketavet solution (12 mg/kg/50 mg/kg). Thrombi were generated in an arteriovenous shunt in which a thrombogenic thread was secured, based on the method described by P C Wong et al. (Wong P C, Crain E J, Nguan O, Watson C A, Racanelli A. Antithrombotic actions of selective inhibitors of blood coagulation factor Xa in rat models of thrombosis Thrombosis Research 1996; 83: 117-126). For this purpose, the left jugular vein and the right carotid artery were exposed. An 8 cm-long polyethylene catheter (PE60, Becton-Dickinson), followed by a 6 cm-long Tygon tube (R-3606, ID 3.2 mm, Kronlab), which contained a roughened, thrombogenic nylon thread (60×0.26 mm, Berkley Trilene) made into a double loop, was secured in the artery. A 2 cm-long polyethylene catheter (PE60, Becton-Dickinson) was secured in the jugular vein and connected via a 6 cm-long polyethylene catheter (PE160, Becton-Dickinson) to the Tygon tube. The tubes were filled with physiological saline solution before the shunt was opened. The extracorporeal circulation was maintained for 15 minutes. The shunt was then removed, and the nylon thread with the thrombus was immediately weighed. The empty weight of nylon thread has been determined before the start of the test. The FXa inhibitor (such as rivaroxaban) was administered to the animals intravenously, and acetylsalicylic acid (ASA) and/or ADP receptor antagonist (such as clopidogrel) was administered orally by gavage before setting up the extracorporeal circulation.

TABLE 1
Synergistic antithrombotic effect of the combination of an
oxazolidinone of the formula (I) with acetylsalicylic acid
and an ADP receptor antagonist

| | Example | | Example 44 + |
|---|---|---|---|
| Substance | 44 | Clopi + ASA | Clopi + ASA |
| [mg/kg] | 0.01 iv | 1 po + 3 po | 0.01 iv + 1 po + |
| | | | 3 po |
| Thrombus reduction | 18 | 20 | 43 |
| [%] | | | |
| | p > 0.05 | p > 0.05 | P < 0.001 |
| | No effect | No effect | Effect |
| [mg/kg] | 0.03 iv | 1 po + 3 po | 0.03 iv + 1 po + |
| | | | 3 po |
| Thrombus weight | 20 | 26 | 43 |
| reduction [%] | | | |
| | p > 0.05 | p < 0.05 | P < 0.001 |
| | No effect | Weak effect | Effect |

Clopi = clopidogrel,
ASA = acetylsalicylic acid (aspirin)

As shown in Table 1, a synergistic effect is achieved with the combination of rivaroxaban (Example 44) with acetylsalicylic acid an ADP receptor blocker such as clopidogrel, i.e. the three components have a mutually potentiating effect. Rivaroxaban (Example 44) in the individual dose is ineffective, and the combination of the two aggregation inhibitors is also ineffective or has only a very weak effect. By contrast, combination of the three compounds leads to a highly significant reduction in the thrombus weight. It is therefore possible by combining an oxazolidinone of the formula (I) with acetylsalicylic acid and an ADP receptor blocker to considerably improve the antithrombotic therapy.

### B Preparation Examples

### Starting Compounds

The preparation of 3-morpholinone is described in U.S. Pat. No. 5,349,045.

The preparation of N-(2,3-epoxypropyl)phthalimide is described in J.-W. Chern et al. Tetrahedron Lett. 1998, 39, 8483.

The substituted anilines can be obtained by reacting, for example, 4-fluoronitrobenzene, 2,4-difluoronitrobenzene or 4-chloronitrobenzene with the appropriate amines or amides in the presence of a base. This can also take place with use of Pd catalysts such as Pd(OAc)₂/DPPF/NaOt-Bu (Tetrahedron Lett. 1999, 40, 2035) or copper (Renger, Synthesis 1985, 856; Aebischer et al., Heterocycles 1998, 48, 2225). Haloaromatic compounds without a nitro group can initially be converted into the corresponding amides in exactly the same way in order to be subsequently nitrated in position 4 (U.S. Pat. No. 3,279,880).

### I. 4-(4-Morpholin-3-onyl)nitrobenzene

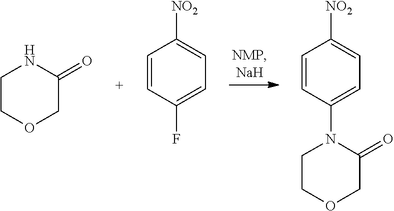 [SMILES: O=C1COCCN1 ; O=[N+]([O-])c1ccc(F)cc1 ; O=C1COCCN1c1ccc([N+](=O)[O-])cc1]

2 mol (202 g) of morpholin-3-one (E. Pfeil, U. Harder, Angew. Chem. 79, 1967, 188) are dissolved in 2 l of N-methylpyrrolidone (NMP). 88 g (2.2 mol) of sodium hydride (60% in paraffin) are then added in portions over a period of 2 h. After hydrogen evolution ceases, 282 g (2 mol) of 4-fluoronitrobenzene are added dropwise while cooling at room temperature over the course of 1 h, and the reaction mixture is then stirred overnight. Subsequently, 1.7 l of the liquid volume are distilled out at 12 mhar and 76° C., the residue is poured into 2 l of water, and this mixture is extracted twice with 1 l of ethyl acetate each time. The combined organic phases are washed with water and then dried over sodium sulfate, and the solvent is distilled off in vacuo. Purification takes place by chromatography on silica gel with hexane/ethyl acetate (1:1) and subsequent crystallization from ethyl acetate. The product is obtained as 78 g of a colorless to brownish solid in 17.6% of theory.

¹H-NMR (300 MHz, CDCl₃): 3.86 (m, 2H, CH₂CH₂), 4.08 (m, 2H, CH₂CH₂), 4.49 (s, 2H, CH₂CO), 7.61 (d, 2H, ³J=8.95 Hz, CHCH), 8.28 (d, 2H, ³J=8.95 Hz, CHCH)

MS (r.I. %)=222 (74, M⁺), 193 (100), 164 (28), 150 (21), 136 (61), 117 (22), 106 (24), 90 (37), 76 (38), 63 (32), 50 (25)

The following compounds were synthesized analogously:
3-fluor-4-(4-morpholin-3-onyl)nitrobenzene
4-(N-piperidonyl)nitrobenzene
3-fluoro-4-(N-piperidonyl)nitrobenzene
4-(N-pyrrolidonyl)nitrobenzene
3-fluoro-4-N-pyrrolidonyl)nitrobenzene

### II. 4-(4-Morpholin-3-onyl)aniline

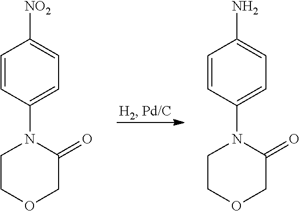 [SMILES: O=C1COCCN1c1ccc([N+](=O)[O-])cc1 ; Nc1ccc(N2CCOCC2=O)cc1]

63 g (0.275 mol) of 4-(4-morpholin-3-onyl)nitrobenzene are dissolved in 200 ml of tetrahydrofuran in an autoclave, 3.1 g of Pd/C (5%) are added, and the mixture is hydrogenated under a hydrogen pressure of 50 bar at 70° C. for 8 h. After filtration of the catalyst, the solvent is distilled out in vacuo and the product is purified by crystallization from ethyl acetate. The product is obtained as 20 g of a colorless to blueish solid in 37.6% of theory.

Purification can also take place by chromatography on silica gel with hexane/ethyl acetate.

¹H-NMR (300 MHz, CDCl₃): 3.67 (m, 2H, CH₂CH₂), 3.99 (m, 2H, CH₂CH₂), 4.27 (s, 2H, CH₂CO), 6.68 (d, 2H, ³J=8.71 Hz, CHCH), 7.03 (d, 2H, ³J=8.71 Hz, CHCH)

MS (r.I. %)=192 (100, M⁺), 163 (48), 133 (26), 119 (76), 106 (49), 92 (38), 67 (27), 65 (45), 52 (22), 28 (22)

The following compounds were synthesized analogously:
3-fluoro-4-(4-morpholin-3-onyl)aniline
4-(N-piperidonyl)aniline
3-fluoro-4-(N-piperidonyl)aniline
4-(N-pyrrolidonyl)aniline
3-fluoro-4-(N-pyrrolidonyl)aniline

General Method for Preparing 4-Substituted Anilines by Reacting 1-fluoro-4-nitrobenzenes and 1-chloro-4-nitrobenzenes with Primary or Secondary Amines and Subsequent Reduction

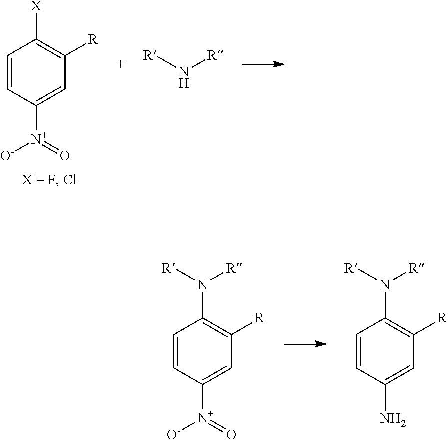

Equimolar amounts of the fluoronitrobenzene or chloronitrobenzene and of the amine are dissolved in dimethyl sulfoxide or acetonitrile (0.1 M to 1 M solution) and stirred at 100° C. overnight. After cooling to RT, the reaction mixture is diluted with ether and washed with water. The organic phase is dried over MgSO₄ filtered and concentrated. If a precipitate is obtained in the reaction mixture, it is filtered off and washed with ether or acetonitrile. If product is also to be found in the mother liquor, this is worked up with ether and water as described. The crude products can be purified by chromatography on silica gel (dichloromethane/cyclohexane and dichloromethane/ethanol mixtures).

For the subsequent reduction, the nitro compound is dissolved in methanol, ethanol or ethanol/dichloromethane mixtures (0.01 M to 0.5 M solution), mixed with palladium on carbon (10%) and stirred under hydrogen of atmospheric pressure overnight. This is followed by filtration and concentration. The crude product can be purified by chromatography on silica gel (dichloromethane/ethanol mixtures) or preparative reversed phase HPLC (acetonitrile/water mixtures).

Alternatively, iron powder can also be used as reducing agent. For this purpose, the nitro compound is dissolved in acetic acid (0.1 M to 0.5 M solution) and, at 90° C., six equivalents of iron powder and water (0.3 to 0.5 times the volume of acetic acid) are added in portions over the course of 10-15 min. After a further 30 min at 90° C., the mixture is filtered and the filtrate is concentrated. The residue is worked up by extraction with ethyl acetate and 2N sodium hydroxide solution. The organic phase is dried over magnesium sulfate, filtered and concentrated. The crude product can be purified by chromatography on silica gel (dichlormethane/ethanol mixtures) or preparative reversed phase HPLC (acetonitrile/water mixtures).

The following starting compounds were prepared in an analogous manner:

### III-1. Tert-butyl 1-(4-aminophenyl)-L-prolinate

MS (ESI): m/z (%)=304 (M+H+MeCN, 100), 263 (M+H, 20);

HPLC (method 4): rt=2.79 min.

### III-2. 1-(4-Aminophenyl)-3-piperidinecarboxamide

MS (ESI): m/z (%)=220 (M+H, 100);

HPLC (method 4): rt=0.59 min.

### III-3. 1-(4-Aminophenyl)-4-piperidinecarboxamide

MS (ESI): m/z (%)=220 (M+H, 100);

HPLC (method 4): rt=0.57 min.

### III-4. 1-(4-Aminophenyl)-4-piperidinone

MS (ESI): m/z (%)=191 (M+H, 100);

HPLC (method 4): rt=0.64 min.

### III-5. 1-(4-Aminophenyl)-L-prolinamide

MS (ESI): m/z (%)=206 (M+H, 100);

HPLC (method 4): rt=0.72 min.

### III-6. [1-(4-Aminophenyl)-3-piperidinyl]methanol

MS (ESI): m/z (%)=207 (M+H, 100);

HPLC (method 4): rt=0.60 min.

### III-.7. [1-(4-Aminophenyl)-2-piperidinyl]methanol

MS (ESI): m/z (%)=207 (M+H, 100);

HPLC (method 4): rt=0.59 min.

### III-8. Ethyl 1-(4-aminophenyl)-2-piperidinecarboxylate

MS (ESI): m/z (%)=249 (M+H, 35), 175 (100);

HPLC (method 4): t=2.43 min.

### III-9. [1-(4-Aminophenyl-2-pyrrolidinyl]methanol

MS (ESI): m/z (%)=193 (M+H, 45):

HPLC (method 4): rt=0.79 min.

### III-10. 4-(2-Methylhexahydro-5H-pyrrolo[3,4-d]isoxazol-5-yl)phenylamine

starting from 2-methylhexahydro-2H-pyrrolo[3,4-d]isoxazole (Ziegler, Carl B., et al.; J.

Heterocycl. Chem.; 25; 2; 1988: 719-723)

MS (ESI): m/z (%)=220 (M+H, 50), 171 (100);

HPLC (method 4): rt=0.54 min.

### III-11. 4-(1-Pyrrolidinyl)-3-(trifluoromethyl)aniline

MS (ESI): m/z (%)=231 (M+H, 100);

HPLC (method 7): rt=3.40 min.

### III-12. 3-Chloro-4-(1-pyrrolidinyl)aniline

MS (ESI): m/z (%)=197 (M+H, 100):

HPLC (method 4): rt=0.78 min.

### III-13. 5-Amino-2-(4-morpholinyl)benzamide

MS (ESI): m/z (%)=222 (M+H, 100);

HPLC (method 4): rt=0.77 min.

### III-14. 3-Methoxy-4-(4-morpholinyl)aniline

MS (ESI): m/z (%)=209 (M+H, 100);

HPLC (method 4): rt=0.67 min.

### III-15. 1-[5-Amino-2-(4-morpholinyl)phenyl]ethanone

MS (ESI): m/z (%)=221 (M+H, 100);

HPLC (method 4): rt=0.77 min.

General Method for Preparing 4-Substituted Anilines by Reacting 1-fluoro-4-nitrobenzenes with Amides and Subsequent Reduction

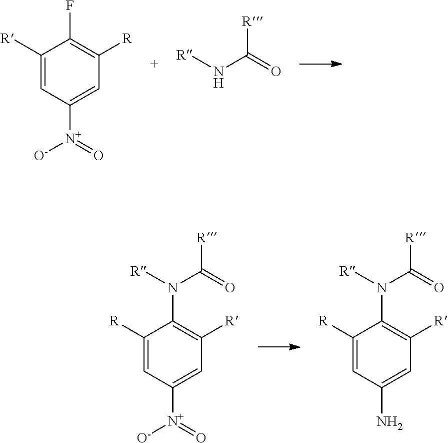

The amide is dissolved in DMF, and 1.5 equivalents of potassium tert-butoxide are added. The mixture is stirred at RT for 1 h, and then 1.2 equivalents of the 1-fluoro-4-nitrobenzene are added in portions. The reaction mixture is stirred at RT overnight, diluted with ether or ethyl acetate and washed with saturated aqueous sodium bicarbonate solution. The organic phase is dried over magnesium sulfate, filtered and concentrated. The crude product can be purified by chromatography on silica gel (dichloromethane/ethanol mixtures).

For the subsequent reduction, the nitro compound is dissolved in ethanol (0.01 M to 0.5 M solution), mixed with palladium on carbon (10%) and stirred under hydrogen of atmospheric pressure overnight. This is followed by filtration and concentration. The crude product can be purified by chromatography on silica gel (dichloromethane/ethanol mixtures) or preparative reversed phase HPLC (acetonitrile/water mixtures).

Alternatively, iron powder can also be used as reducing agent. For this purpose, the nitro compound is dissolved in acetic acid (0.1 M to 0.5 M solution) and, at 90° C., six equivalents of iron powder and water (0.3 to 0.5 limes the volume of acetic acid) are added in portions over the course of 10-15 min. After a further 30 min at 90° C., the mixture is filtered and the filtrate is concentrated. The residue is worked up by extraction with ethyl acetate and 2N sodium hydroxide solution. The organic phase is dried over magnesium sulfate, filtered and concentrated. The crude product can be purified by chromatography on silica gel (dichlormethane/ethanol mixtures) or preparative reversed phase HPLC (acetonitrile/water mixtures).

The following starting compounds were prepared in an analogous manner:

### IV-1. 1-[4-Amino-2-(trifluoromethyl)phenyl]-2-pyrrolidinone

MS (ESI): m/z (%)=245 (M+H, 100):

HPLC (method 4): rt=2.98 min

### IV-2. 4-[4-Amino-2-(trifluoromethyl)phenyl]-3-morpholinone

MS (ESI): m/z (%)=261 (M+H, 100);

HPLC (method 4): rt=2.54 min.

### IV-3, 4-(4-Amino-2-chlorophenyl)-3-morpholinone

MS (ESI): m/z (%)=227 (M+H, 100);

HPLC (method 4): rt=1.96 min.

### IV-4. 4-(4-Amino-2-methylphenyl)-3-morpholinone

MS (ESI): m/z (%)=207 (M+H, 100);

HPLC (method 4): rt=0.71 min.

### IV-5. 5-Amino-2-(3-oxo-4-morpholinyl)benzonitrile

MS (ESI): m/z (%)=218 (M+H, 100);

HPLC (method 4): rt=1.85 min.

### IV-6. 1-(4-Amino)-2-chlorophenyl-2-pyrrolidinone

MS (ESI): m/z (%)=211 (M+H, 100);

HPLC (method 4): rt=2.27 min.

### IV-7. 4-(4-Amino-2,6-dimethylphenyl)-3-morpholinone

starting from 2-fluoro-1,3-dimethyl-5-nitrobenzene (Bartoli et al., J. Org. Chem. 1975, 40, 872):

MS (ESI): m/z (%)=221 (M+11, 100):

HPLC (method 4): rt=0.77 min.

### IV-8. 4-(2,4-Diaminophenyl)-3-morpholinone

starting from 1-fluoro-2,4-dinitrobenzene:

MS (ESI): m/z (%)=208 (M+H, 100);

HPLC (method 4): rt=0.60 min.

### IV-9. 4-(4-Amino-2-chlorophenyl)-2-methyl-3-morpholine

starting from 2-methyl-3-morpholinone (Pfeil. E.; Harder, U.; Angew. Chem. 1967, 79, 188):

MS (ESI): m/z (%)=241 (M+1H, 100);

HPLC (method 4): rt=2.27 min.

### IV-10, 4-(4-Amino-2-chlorophenyl-6-methyl-3-morpholinone

starting from 6-methyl-3-morpholinone (EP 350 (2):

MS (ESI): m/z (%)=241 (M+H, 100);

HPLC (method 4): rt=2.43 min.

### SYNTHESIS EXAMPLES

The following Examples 1 to 13, 17 to 19 and 36 to 57 relate to process variant [A].

### Example 1

### Preparation of 5-chloro-N-{[(5S)-3-(3-fluoro-4-morpholinophenyl)-2-oxo-1,3-oxazolidin-5-yl]methyl}-2-thiophenecarboxamide

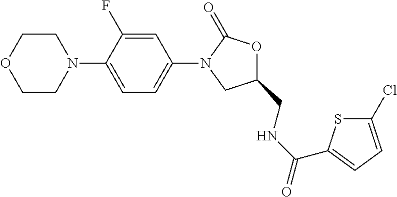 [SMILES: O=C(NC[C@H]1CN(c2ccc(N3CCOCC3)c(F)c2)C(=O)O1)c1ccc(Cl)s1]

(5S)-5-(Aminomethyl)-3-(3-fluoro-4-morpholinophenyl)-1,3-oxazolidin-2-one (for preparation, see S. J. Brickner et al., J. Med. Chem. 1996, 39, 673) (0.45 g, 1.52 mmol), 5-chlorothiophene-2-carboxylic acid (0.25 g, 1.52 mmol) and 1-hydroxy-1H-benzotriazole hydrate (HOBT) (0.3 g, 1.3 equivalents) are dissolved in 9.9 ml of DMF. 0.31 g (1.98 mmol, 1.3 equivalents) of N′-(3-dimethylaminopropyl)-N-ethylcarbodiimide (EDCI) is added and, at room temperature, 0.39 g (0.53 ml, 3.05 mmol, 2 equivalents) of diisopropylethylamine (DIEA) is added dropwise. The mixture is stirred at room temperature overnight. 2 g of silica gel are added and the mixture is evaporated to dryness in vacuo. The residue is chromatographed on silica gel with a toluene/ethyl acetate gradient. 0.412 g (61.5% of theory) of the target compound is obtained with a melting point (m.p.) of 197° C.

R_{f} (SiO₂, toluene/ethyl acetate 1:1)=0.29 (precursor=0.0);

MS (DCI) 440.2 (M+H), Cl pattern;

¹H-NMR (d₆-DMSO, 300 MHz) 2.95 (m, 4H), 3.6 (t, 2H), 3.72 (m, 4H), 3.8 (dd, 1H), 4.12 (t, 1H), 4.75-4.85 (m, 1H), 7.05 (t, 1H), 7.15-7.2 (m, 3H), 7.45 (dd, 1H), 7.68 (d, 1H), 8.95 (t, 1H).

### Example 2

### 5-Chloro-N-{[(5S)-3-(4-morpholinophenyl-2-oxo-1,3-oxazolidin-5-yl]methyl}-2-thiophenecarboxamide

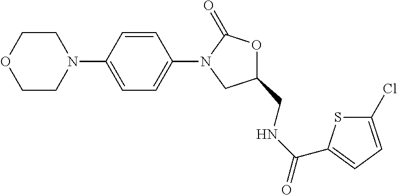 [SMILES: O=C(NC[C@H]1CN(c2ccc(N3CCOCC3)cc2)C(=O)O1)c1ccc(Cl)s1]

is obtained analogously from benzyl 4-morpholinophenylcarbamate via the stage of (5S)-5-(aminomethyl)-3-(3-fluoro-4-morpholinophenyl)-1,3-oxazolidin-2-one (see Example 1).

M.p.: 198° C.;

IC₅₀=43 nM;

Rₜ (SiO₂, toluene/ethyl acetate 1:1)=0.24.

### Example 3

### 5-Chloro-N-({(5S)-3-[3-fluoro 4-(1,4-thiazinan-4-yl)phenyl]-2-oxo-1,3-oxazolidin-5-yl}methyl)-2-thiophenecarboxamide

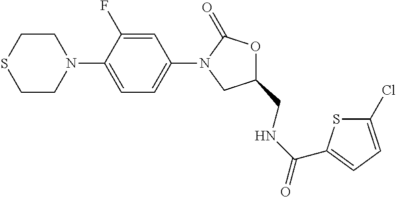 [SMILES: O=C(NC[C@H]1CN(c2ccc(N3CCSCC3)c(F)c2)C(=O)O1)c1ccc(Cl)s1]

is obtained analogously from (5S)-5-(aminomethyl)-3-[3-fluoro-4-(1,4-thiazinan-4-yl)phenyl]-1,3-oxazolidin-2-one (for preparation, see M. R. Barbachyn et al., J. Med. Chem. 1996, 39, 680).

M.p.: 193° C.;

Yield: 82%;

R_{f} (SiO₂, toluene/ethyl acetate 1:1)=0.47 (precursor=0.0).

### Example 4

### 5-Bromo-N-({(5S)-3-[3-fluoro-4-(1,4-thiazinan-4-yl)phenyl]-2-oxo-1,3-oxazolidin-5-yl}methyl)-2-thiophenecarboxamide

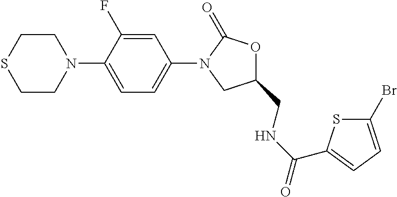 [SMILES: O=C(NC[C@H]1CN(c2ccc(N3CCSCC3)c(F)c2)C(=O)O1)c1ccc(Br)s1]

is obtained analogously from 5-bromothiophene-2-carboxylic acid.

M.p.: 200° C.

### Example 5

### N-({(5S)-3-[3-Fluoro-4-(1,4-thiazinan-4-yl)phenyl]-2-oxo-1,3-oxazolidin-5-yl}methyl)-5-methyl-2-thiophenecarboxamide

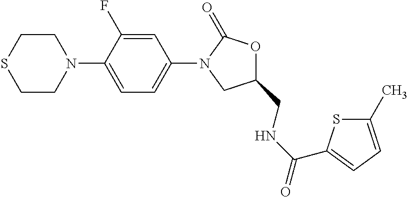 [SMILES: Cc1ccc(C(=O)NC[C@H]2CN(c3ccc(N4CCSCC4)c(F)c3)C(=O)O2)s1]

is obtained analogously from 5-methylthiophene-2-carboxylic acid.

M.p.: 167° C.

### Example 6

### 5-Chloro-N-{[(5S)-3-(6-methylthieno[2,3-b]pyridin-2-yl)-2-oxo-1,3-oxazolidin-5-yl]methyl}-2-thiophenecarboxamide

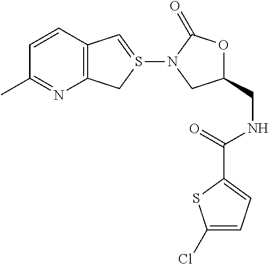 [SMILES: Cc1ccc2c(n1)CS(N1C[C@H](CNC(=O)c3ccc(Cl)s3)OC1=O)=C2]

is obtained analogously from (5S)-5-(aminomethyl)-3-(6-methylthieno[2,3-b]pyridin-2-yl)-1,3-oxazolidin-2-one (for preparation, see EP-A-785 200).

M.p.: 247° C.

### Example 7

### 5-Chloro-N-{[(5S)-3-(3-methyl-2-oxo-2,3-dihydro-1,3-benzothiazol-6-yl)-2-oxo-1,3-oxazolidin-3-yl]methyl}-2-thiophenecarboxamide

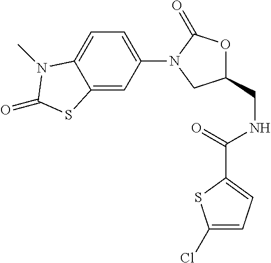 [SMILES: Cn1c(=O)sc2cc(N3C[C@H](CNC(=O)c4ccc(Cl)s4)OC3=O)ccc21]

is obtained analogously from 6-[(5S)-5 (aminomethyl)-2-oxo-1,3-oxazolidin-3-yl]-3-methyl-1,3-benzothiazol-2(3H)-one (for preparation, see EP-A-738 726).

M.p.: 217° C.

### Example 8

### 5-Chloro-N-[((5S)-3-{3-fluoro-4-[4-(4-pyridinyl)piperazino]phenyl}-2-oxo-1,3-oxazolidin-5-yl)methyl]-2-thiophenecarboxamide

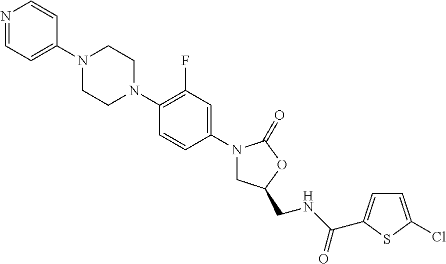 [SMILES: O=C(NC[C@H]1CN(c2ccc(N3CCN(c4ccncc4)CC3)c(F)c2)C(=O)O1)c1ccc(Cl)s1]

is obtained analogously from (5S)-5-(aminomethyl)-3-[3-fluoro-4-[4-(4 pyridinyl)piperazino]phenyl]-1,3-oxazolidin-2-one (preparation in analogy to J. A. Tucker et al., J. Med. Chem. 1998, 41, 3727).

MS (ESI) 516 (M+H), Cl pattern.

### Example 9

### 5-Chloro-N-({(5S)-3-[3-fluoro-4-(4-methylpiperazino)phenyl]-2-oxo-1,3-oxazolidin-5-yl}methyl)-2-thiophenecarboxamide

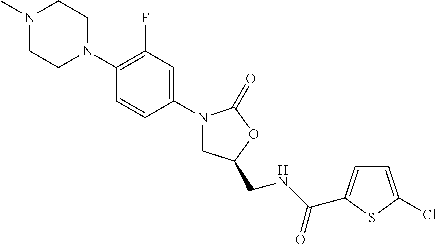 [SMILES: CN1CCN(c2ccc(N3C[C@H](CNC(=O)c4ccc(Cl)s4)OC3=O)cc2F)CC1]

is obtained analogously from (5S)-5-(aminomethyl)-3-[3-fluoro-4-(4-methylpiperazino)phenyl]-1,3-oxazolidin-2-one.

### Example 10

### 5-Chloro-N-({(5S)-3-[3-fluoro-4-(4-tert-butoxycarbonylpiperazin-1-yl)phenyl]-2-oxo-1,3-oxazolidin-5-yl}methyl)-2-thiophenecarboxamide

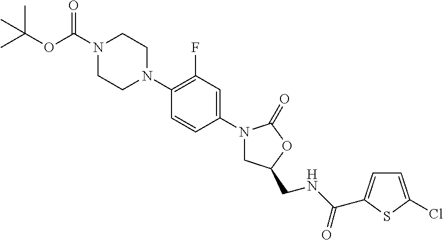 [SMILES: CC(C)(C)OC(=O)N1CCN(c2ccc(N3C[C@H](CNC(=O)c4ccc(Cl)s4)OC3=O)cc2F)CC1]

is obtained analogously from (5S)-5-(aminomethyl)-3-[3-fluoro-4-(4-tert-butoxycarbonylpiperazin-1-yl)phenyl]-1,3-oxazolidin-2-one (for preparation, see WO-A-93/23384 which has already been cited).

M.p.: 184° C.;

R_{f} (SiO₂, toluene/ethyl acetate 1:1)=0.42.

### Example 11

### 5-Chloro-N-({(5S)-3-[3-fluoro-4-(piperazin-1-yl)phenyl]-2-oxo-1,3-oxazolidin-5-yl}methyl)-2-thiophenecarboxamide

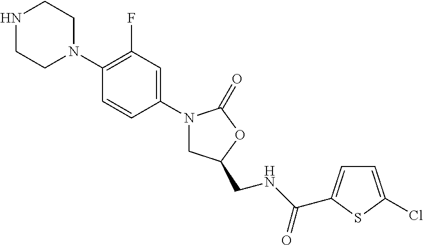 [SMILES: O=C(NC[C@H]1CN(c2ccc(N3CCNCC3)c(F)c2)C(=O)O1)c1ccc(Cl)s1]

is obtained by reacting Example 12 with trifluoroacetic acid in methylene chloride.

IC₅₀=140 nM;

¹H-NMR [d₆-DMSO]: 3.01-3.25 (m, 8H), 3.5-3.65 (m, 2H), 3.7-3.9 (m, 1H), 4.05-4.2 (m, 1H), 4.75-4.9 (m, 1H), 7.05-7.25 (m, 3H), 7.5 (dd, 1H), 7.7 (d, 1H), 8.4 (broad s, 1H), 9.0 (t, 1H).

### Example 12

### 5-Chloro-N-[((5S)-3-(2,4′-bipyridinyl-5-yl)-2-oxo-1,3-oxazolidin-5-yl)methyl]-2-thiophenecarboxamide

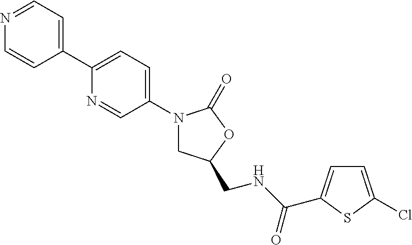 [SMILES: O=C(NC[C@H]1CN(c2ccc(-c3ccncc3)nc2)C(=O)O1)c1ccc(Cl)s1]

is obtained analogously from (5S)-5-aminomethyl-3-(2,4′-bipyridinyl-5-yl)-2-oxo-1, 3-oxazolidin-2-one (for preparation, see EP-A-789 026).

R_{f} (SiO₂, ethyl acetate/ethanol 1:2)=0.6;

MS (ESI) 515 (M+H), Cl pattern.

### Example 13

### 5-Chloro-N-{[(5S)-2-oxo-3-(4-piperidinophenyl)-1,3-oxazolidin-5-yl]methyl}-2-thiophenecarboxamide

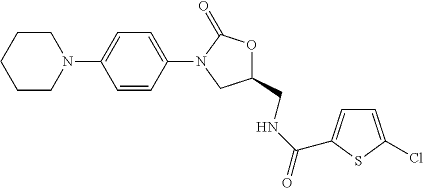 [SMILES: O=C(NC[C@H]1CN(c2ccc(N3CCCCC3)cc2)C(=O)O1)c1ccc(Cl)s1]

is obtained from 5-(hydroxymethyl)-3-(4-piperidinophenyl)-1,3-oxazolidin-2-one (for preparation, see DE 2708236) after mesylation, reaction with potassium phthalimide, hydrazinolysis and reaction with 5-chlorothiophene-2-carboxylic acid.

R_{f} (SiO₂, ethyl acetate/toluene 1:1)=0.31;

M.p. 205° C.

### Example 17

### 5-Chloro-N-({(5S)-2-oxo-3-[4-(2-oxo-1-pyrrolidinyl)phenyl]-1,3-oxazolidin-5-yl}methyl)-2-thiophenecarboxamide

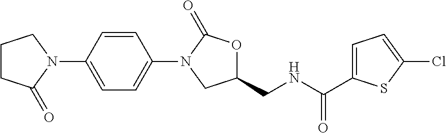 [SMILES: O=C(NC[C@H]1CN(c2ccc(N3CCCC3=O)cc2)C(=O)O1)c1ccc(Cl)s1]

5-Chloro-N-({(5S)-2-oxo-3-[4-(2-oxo-1-pyrrolidinyl)phenyl]-1,3-oxazolidin-5-yl}methyl)-2-thiophenecarboxamide is obtained from 1-(4-aminophenyl)pyrrolidin-2-one (for preparation, see Reppe et al., Justus Liebigs Ann. Chem.; 596; 1955; 209) in analogy to the known synthesis scheme (see S. J. Brickner et al., J. Med. Chem. 1996, 39, 673) after reaction with benzyloxycarbonyl chloride, subsequent reaction with R-glycidyl butyrate, mesylation, reaction with potassium phthalimide, hydrazinolysis in methanol and finally reaction with 5-chlorothiophene-2-carboxylic acid. The 5-chloro-N-({(5S)-2-oxo-3-[4-(2-oxo-1-pyrrolidinyl)phenyl]-1,3-oxazolidin-5-yl}methyl)-2-thiophenecarboxamide obtained in this way has an IC₅₀ of 4 nM (test method for the IC⁵⁰ according to Example A-1, a.1) “Measurement of factor Xa inhibition” described above).

M.p.: 229° C.:

R_{f} (SiO₂, toluene/ethyl acetate 1:1)=0.05 (precursor: =0.0);

MS (ESI): 442.0 (21%. M+Na, Cl pattern), 420.0 (72%, M+H, Cl pattern), 302.3 (12%), 215 (52%), 145 (100%);

¹H-NMR (d₆-DMSO, 300 MHz); 2.05 (m, 2H), 2.45 (m, 2H), 3.6 (t, 2H), 3.77-3.85 (m, 3H), 4.15 (t, 1H), 4.75-4.85 (m, 1H), 7.2 (d, 1H), 7.5 (d, 2H), 7.65 (d, 2H), 7.69 (d, 1H), 8.96 (t, 1H).

The individual stages in the synthesis of Example 17 described above, with the respective precursors, are as follows:

4.27 g (25.03 mmol) of benzyl chloroformate are slowly added to 4 g (22.7 mmol) of 1-(4-aminophenyl)pyrrolidin-2-one and 3.6 ml (28.4 mmol) of N,N-dimethylaniline in 107 ml of tetrahydrofuran at −20° C. The mixture is stirred at −20° C. for 30 minutes and then allowed to reach room temperature. 0.5 l of ethyl acetate is added, and the organic phase is washed with 0.5 l of saturated NaCl solution. The removed organic phase is dried with MgSO₄, and the solvent is evaporated in vacuo. The residue is triturated with diethyl ether and filtered off with suction. 5.2 g (73.8% of theory) of benzyl 4-(2-oxo-1-pyrrolidinyl)phenylcarbamate are obtained as pale beige crystals with a melting point of 174° C.

7.27 ml of a 2.5 M solution of n-butyllithium (BuLi) in hexane are added dropwise to 1.47 g (16.66 mmol) of isoamyl alcohol in 200 ml of tetrahydrofuran under argon at −10° C., a further 8 ml of the BuLi solution being necessary until the color of the added N-benzylidenbenzylamine indicator changed. The mixture is stirred at −10° C. for 10 minutes and cooled to −78° C., and a solution of 4.7 g (15.14 mmol) of benzyl 4-(2-oxo-1-pyrrolidinyl)phenylcarbamate is slowly added. Then a further 4 ml of n-BuLi solution are added until the color of the indicator changes to pink. The mixture is stirred at −78° C. for 10 minutes and 2.62 g (18.17 mmol) of R-glycidyl butyrate are added, and the mixture is stirred at −78° C. for 30 minutes.

The mixture is allowed to reach room temperature overnight, 200 ml of water are added to the mixture, and the THF content is evaporated in vacuo. The aqueous residue is extracted with ethyl acetate, and the organic phase is dried with MgSO₄ and concentrated in vacuo. The residue is triturated with 500 ml of diethyl ether, and the crystals which have separated out are filtered off with suction in vacuo.

3.76 g (90% of theory) of (5R)-5-(hydroxymethyl)-3-[4-(2-oxo-1-pyrrolidinyl)phenyl]-1,3-oxazolidin-2-one are obtained with a melting point of 148° C. and an R_{f} (SiO₂, toluene/ethyl acetate 1:1)=0.04 (precursor=0.3).

3.6 g (13.03 mmol) of (5R)-5-(hydroxymethyl)-3-[4-(2-oxo-1-pyrrolidinyl)phenyl]-1,3-oxazolidin-2-one and 2.9 g (28.67 mmol) of triethylamine are introduced into 160 ml of dichloromethane at 0° C. with stirring. 1.79 g (15.64 mmol) of methanesulfonyl chloride are added with stirring, and the mixture is stirred at 0° C. for 1.5 hours and at room temperature for 3 h.

The reaction mixture is washed with water and the aqueous phase is extracted once more with methylene chloride. The combined organic extracts are dried with MgSO₄ and evaporated. The residue (1.67 g) is then dissolved in 70 ml of acetonitrile, 2.62 g (14.16 mmol) of potassium phthalimide are added, and the mixture is stirred in a closed vessel at 180° C. in a microwave oven for 45 minutes.

The mixture is filtered off from the insoluble residue, the filtrate is concentrated in vacuo, the residue (1.9 g) is dissolved in methanol, and 0.47 g (9.37 mmol) of hydrazine hydrate is added. The mixture is boiled for 2 hours and cooled, saturated sodium bicarbonate solution is added, and the mixture is extracted six times with a total of 2 l of methylene chloride. The combined organic extracts of the crude (5S)-5-(aminomethyl)-3-[4-(2-oxo-1-pyrrolidinyl)phenyl]-1,3-oxazolidin-2-one are dried with MgSO₄ and concentrated in vacuo.

The final stage, 5-chloro-N-({5S)-2-oxo-3-[4-(2-oxo-1-pyrrolidinyl)phenyl]-1,3-oxazolidin-5-yl}methyl)-2-thiophenecarboxamide, is prepared by dissolving 0.32 g (1.16 mmol) of the (5S)-5-(aminomethyl)-3-[4-(2-oxo-1-pyrrolidinyl)phenyl]-1,3-oxazolidin-2-one prepared above, 5-chlorothiophene-2-carboxylic acid (0.19 g; 1.16 mmol) and 1-hydroxy-1H-benzotriazole hydrate (HOBT) (0.23 g, 1.51 mmol) in 7.6 ml of DMF. 0.29 g (1.51 mmol) of N′-(3-dimethylaminopropyl)-N-ethylcarbodiimide (EDCI) is added and, at room temperature, 0.3 g (0.4 ml; 2.32 mmol, 2 equivalents) of diisopropylethylamine (DIEA) is added dropwise. The mixture is stirred at room temperature overnight.

The mixture is evaporated to dryness in vacuo, and the residue is dissolved in 3 ml of DMSO and chromatographed on an RP-MPLC with acetonitrile/water/0.5% TFA gradients. The acetonitrile content is evaporated off from the appropriate fractions, and the precipitated compound is filtered off with suction. 0.19 g (39% of theory) of the target compound is obtained.

The following were prepared in an analogous manner:

### Example 18

### 5-Chloro-N-({(5S)-2-oxo-3-[4-(1-pyrrolidinyl)phenyl]-1,3-oxazolidin-5-yl}methyl)-2-thiophencarboxamide

The compound 5-chloro-N-({(5S)-2-oxo-3-[4-(1-pyrrolidinyl)phenyl]-1,3-oxazolidin-5-yl}methyl)-2-thiophenecarboxamide is obtained from 4-pyrrolidin-1-ylaniline (Reppe et al., Justus Liebigs Ann. Chem.; 596; 1955; 151) in analogy to Example 17.

IC₅₀=40 nM;

M.p.: 216° C.;

R_{f} (SiO₂, toluene/ethyl acetate 1:1)=0.31 [precursor: =0.0].

### Example 19

### 5-Chloro-N-({(5S)-2-oxo-3-[4-(diethylamino)phenyl]-1,3-oxazolidin-5-yl}methyl)-2-thiophenecarboxamide

The compound 5-chloro-N-({(5S)-2-oxo-3-[4-(diethylamino)phenyl]-1,3-oxazolidin-5-yl}methyl)-2-thiophenecarboxamide is obtained analogously from N,N-diethylphenyl-1,4-diamine (U.S. Pat. No. 2,811,555; 1955).

IC₅₀=270 nM;

M.p.: 181° C.

R_{f} (Si %, toluene/ethyl acetate 1:1)=0.25 [precursor: =0.0].

### Example 36

### 5-Chloro-N-({(5S)-3-[2-methyl-4-(4-morpholinyl)phenyl]-2-oxo-1,3-oxazolidin-5-yl}methyl)-2-thiophenecarboxamide

starting from 2-methyl-4-(4-morpholinyl)aniline (J. E. LuValle et al. J. Am. Chem. Soc. 1948, 70, 2223):

MS (ESI): m/z (%)=436 ([M+H]⁺, 100), Cl pattern;

HPLC (method 1); rt (%)=3.77 (98).

IC₅₀: 1.26 μM

### Example 37

### 5-Chloro-N-{[(5S)-3-(3-chloro-4-morpholinophenyl)-2-oxo-1,3-oxazolidin-5-yl]methyl}-2-thiophenecarboxamide

starting from 3-chloro-4-(4-morpholinyl)aniline (H. R. Snyder et al. J. Pharm. Sci. 1977, 66, 1204):

MS (ESI): m/z (%)=456 ([M+H]⁺, 100), Cl₂ pattern;

HPLC (method 2): rt (%)=4.31 (100).

IC₅₀: 33 nM

### Example 38

### 5-Chloro-N-({5S)-3-[4-(4-morpholinylsulfonyl)phenyl]-2-oxo-1,3-oxazolidin-5-yl}methyl)-2-thiophenecarboxamide

starting from 4-(4-morpholinylsulfonyl)aniline (Adams et al. J. Am. Chem. Soc. 1939, 61, 2342):

MS (ESI): m/z (%)=486 ([M+H]⁺, 100), (Cl pattern;

HPLC (method 3): rt (%)=4.07 (100).

IC₅₀:2 μM

### Example 39

### 5-Chloro-N-({(5S)-3-[4-(1-azetidinylsulfonyl)phenyl]-2-oxo-1,3-oxazolidin-5-yl}methyl)-2-thiophenecarboxamide

starting from 4-(1-azetidinylsulfonyl)aniline:

MS (DCI, NH₃): m/z (%)=473 ([M+NH₄]⁺, 100), Cl pattern;

HPLC (method 3): rt (%)=4.10 (100).

IC₅₀: 0.84 μM

### Example 40

### 5-Chloro-N-[((5S)-3-{4-[(dimethylamino)sulfonyl]phenyl}-2-oxo-1,3-oxazolidin-5-yl)methyl]-2-thiophenecarboxamide

starting from 4-amino-N,N-dimethylbenzenesulfonamide (I. K. Khanna et al. J. Med. Chem. 1997, 40, 1619):

MS (ESI): m/z (%)=444 ([M+H]⁺, 100), Cl pattern;

HPLC (method 3): rt (%)=4.22 (100).

IC₅₀: 90 nM

General Method for the Acylation of 5-(aminomethyl)-3-[4-(2-oxo-1-pyrrolidinyl)phenyl]-1,3-oxazolidin-2-one with carbonyl chlorides.

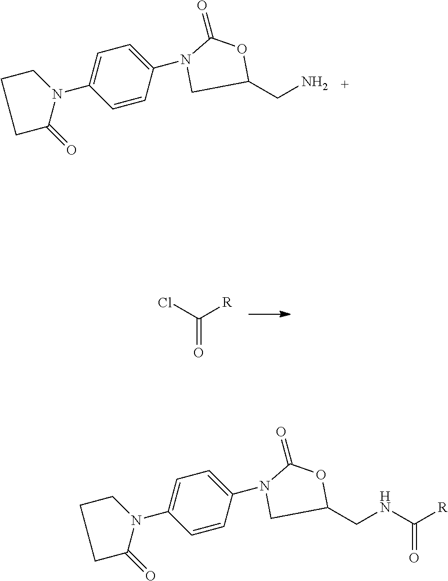

An approx. 0.1 molar solution of 5-(aminomethyl)-3-[4-(2-oxo-1-pyrrolidinyl)phenyl]-1,3-oxazolidin-2-one (from Example 45) (1.0 eq.) and absolute pyridine (approx. 6 q) in absolute dichloromethane is added dropwise to the appropriate acid chloride (2.5 eq.) under argon at room temperature. The mixture is stirred at room temperature for about 4 h before approx. 5.5 eq of PS-trisamine (Argonaut Technologies) are added. The suspension is stirred gently for 2 h and, after dilution with dichloromethane/DMF (3:1), filtered (the resin is washed with dichloromethane/DMF) and the filtrate is concentrated. The resulting product is purified by preparative RP-HPLC where appropriate.

The following was prepared in an analogous manner:

### Example 41

### N-({2-oxo-3-[4-(2-oxo-1-pyrrolidinyl)phenyl]-1,3-oxazolidin-5-yl}methyl)-2-thiophenecarboxamide

LC-MS (method 6): m/z (%)=386 (M+H, 100);

LC-MS: rt (%)=3.04 (100).

IC₅₀: 1.3 μM

General Method for Preparing Acyl Derivatives Starting from 5-(aminomethyl)-3-[4-(2-oxo-1-pyrrolidinyl)phenyl]-1,3-oxazolidin-2-one and carboxylic acids

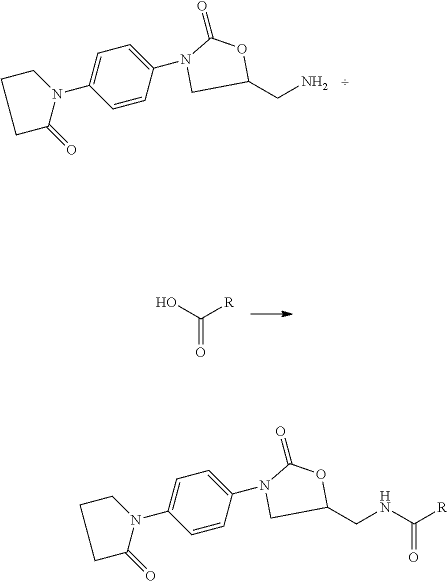

The appropriate carboxylic acid (approx. 2 eq) and a mixture of absolute dichloromethane/DMF (approx. 9:1) are added to 2.9 eq. of resin-bound carbodiimide (PS-Carbodiimide, Argonaut Technologies). After shaking gently at room temperature for about 15 min, 5-(aminomethyl)-3-[4-(2-oxo-1-pyrrolidinyl)phenyl]-1,3-oxazolidin-2-one (from Example 45) (1.0 eq.) is added, and the mixture is shaken overnight before the resin is filtered off (washing with dichloromethane) and the filtrate is concentrated. The resulting product is purified by preparative RP-HPLC where appropriate.

The following were prepared in an analogous manner:

### Example 42

### 5-Methyl-N-({2-oxo-3-[4-(2-oxo-1-pyrrolidinyl)phenyl]-1,3-oxazolidin-5-yl}methyl)-2-thiophenecarboxamide

LC-MS: m/z (%)=400 (M+H, 100);

LC-MS (method 6): rt (%)=3.23 (100).

IC₅₀: 0.16 μM

### Example 43

### 5-Bromo-N-({2-oxo-3-[4-(2-oxo-1-pyrrolidinyl)phenyl]-1,3-oxazolidin-5-yl}methyl)-2-thiophenecarboxamide

LC-MS: m/z (%)=466 (M+1H, 100);

LC-MS (method 5): rt (%)=3.48 (78).

IC₅₀: 0.014 μM

### Example 44

### 5-Chloro-N-({(5S)-2-oxo-3-[4-(3-oxo-4-morpholinyl)phenyl]-1,3-oxazolidin-5-yl}methyl)-2-thiophencarboxamide

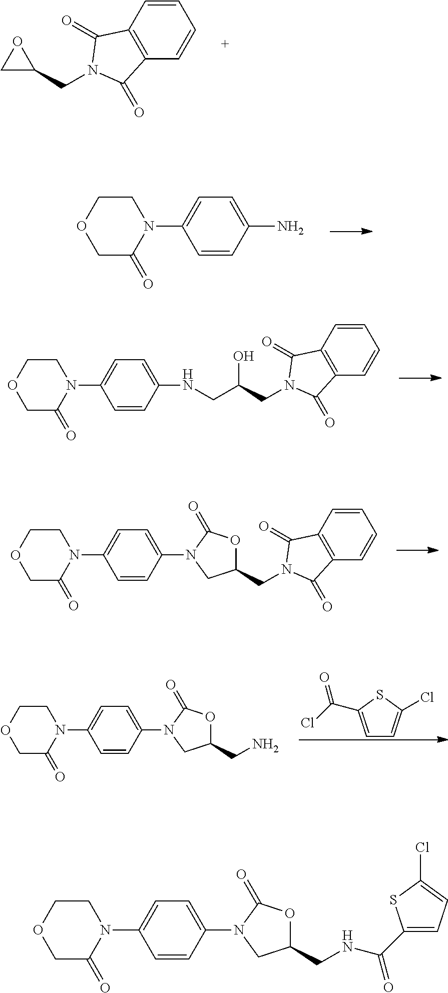

### a) 2-((2R)-2-Hydroxy-3-{[4-(3-oxo-4-morpholinyl)phenyl]amino}propyl)-1H-isoindole-1,3(2H)-dione

A suspension of 2-[(2S)-2-oxiranylmethyl]-1H-isoindole-1,3(2H)-dione (A. Gutcait et al. Tetrahedron Asym. 1996, 7, 1641) (5.68 g, 27.9 mmol) and 4-(4-aminophenyl)-3-morpholinone (5.37 g, 27.9 mmol) in ethanol/water (9:1, 140 ml) is refluxed for 14 h (the precipitate dissolves and, after some time, there is renewed formation of a precipitate). The precipitate (desired product) is filtered off, washed three times with diethyl ether and dried. The combined mother liquors are concentrated in vacuo and, after addition of a second portion of 2-[(2S)-2-oxiranylmethyl]-1H-isoindole-1,3(2H)-dione (2.84 g, 14.0 mmol), are suspended in ethanol/water (9:1, 70 ml) and refluxed for 13 h (the precipitate dissolves and, after some time, there is renewed formation of a precipitate). The precipitate (desired product) is filtered off, washed three times with diethyl ether and dried. Overall yield: 10.14 g, 92% of theory.

MS (ESI): m/z (%)=418 ([M+Na]⁺, 84), 396 ([M+H]⁺, 93);

HPLC (method 3): rt (%)=3.34 (100).

### b) 2-({(5S)-2-Oxo-3-[4-(3-oxo-4-morpholinyl)phenyl]-1,3-oxazolidin-5-yl}methyl)-1H-isoindole-1,3(2H)-dione

N,N′-Carbonyldiimidazole (2.94 g, 18.1 mmol) and dimethylaminopyridine (catalytic amount) are added to a suspension of the amino alcohol (3.58 g, 9.05 mmol) in tetrahydrofuran (90 ml) under argon at room temperature. The reaction suspension is stirred at 60° C. for 12 h (the precipitate dissolves and, after some time, there is renewed formation of a precipitate), a second portion of N,N′-carbonyldiimidazole (2.94 g, 18.1 mmol) is added, and the mixture is stirred at 60° C. for a further 12 h. The precipitate (desired product) is filtered off, washed with tetrahydrofuran and dried. The filtrate is concentrated in vacuo and further product is purified by flash chromatography (dichloromethane/methanol mixtures). Overall yield: 3.32 g, 87% of theory.

MS (ESI): m/z (%)=422 ([M+H]⁺, 100);

HPLC (method 4): rt (%)=3.37 (100).

### c) 5-Chloro-N-({(5S)-2-oxo-3-[4-(3-oxo-4-morpholinyl)phenyl]-1,3-oxazolidin-5-yl}methyl)-2-thiophenecarboxamide

Methylamine (40% strength in water, 10.2 ml, 0.142 mol) is added dropwise to a suspension of the oxazolidinone (4.45 g, 10.6 mmol) in ethanol (102 ml) at room temperature. The reaction mixture is refluxed for 1 h and concentrated in vacuo. The crude product is employed without further purification in the next reaction.

5-Chlorothiophene-2-carbonyl chloride (2.29 g, 12.7 mmol) is added dropwise to a solution of the amine in pyridine (90 ml) under argon at 0° C. The ice cooling is removed and the reaction mixture is stirred at room temperature for 1 h, and water is added. Addition of dichloromethane and phase separation are followed by extraction of the aqueous phase with dichloromethane. The combined organic phases are dried (sodium sulfate), filtered and concentrated in vacuo. The desired product is purified by flash chromatography (dichloromethane/methanol mixtures). Overall yield: 3.92 g, 86% of theory.

M.p.: 232-233° C.;

¹H NMR (DMSO-d⁶, 200 MHz): 9.05-8.90 (t, J=5.8 Hz, 1H), 7.70 (d, J=4.1 Hz, 1H), 7.56 (d, J=9.0 Hz, 2H), 7.41 (d, J=9.0 Hz, 2H), 7.20 (d, J=4.1 Hz, 1H), 4.93-4.75 (m, 1H), 4.27-4.12 (m, 3H), 4.02-3.91 (m, 2H), 3.91-3.79 (dd, J=6.1 Hz, 9.2 Hz, 1H), 3.76-3.66 (m, 2H), 3.66-3.54 (m, 2H);

MS (ESI): m/z (%)=436 ([M+H]⁺, 100, Cl pattern):

HPLC (method 2): rt (%)=3.60 (100):

[α]²³_{D}=−38° (c 0.2985, DMSO); ee: 99%.

IC₅₀: 0.7 nM

The following were prepared in an analogous manner:

### Example 45

### 5-Methyl-N-({(5S)-2-oxo-3-[4-(3-oxo-4-morpholinyl)phenyl]-1,3-oxazolidin-5-yl}methyl)-2-thiophenecarboxamide

MS (ESI): m/z (%)=831 ([2M+H]⁺, 100), 416 ([M+H]⁺, 66);

HPLC (method 3): rt (%)=3.65 (100).

IC₅₀: 4.2 nM

### Example 46

### 5-Bromo-N-({(5S)-2-ox-3-[4-(3-oxo-4-morpholinyl)phenyl]-1,3-oxazolidin-5-yl}methyl)-2-thiophenecarboxamide

MS (ESI): m/z (%)=480 ([M+H]⁺, 100, Br pattern);

HPLC (method 3): rt (%)=3.87 (100).

IC₅₀: 0.3 nM

### Example 47

### 5-Chloro-N-{[(5S)-3-(3-isopropyl-2-oxo-2,3-dihydro-1,3-benzoxazol-6-yl)-2-oxo-1,3-oxazolidin-5-yl]methyl}-2-thiophenecarboxamide

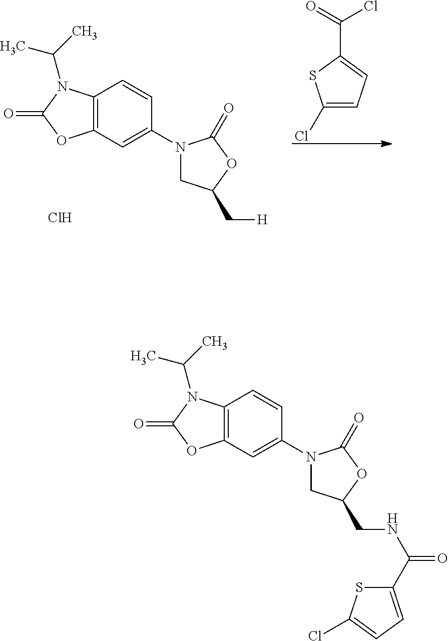 [SMILES: CC(C)n1c(=O)oc2cc(N3C[C@H](C)OC3=O)ccc21 ; Cl ; O=C(Cl)c1ccc(Cl)s1 ; CC(C)n1c(=O)oc2cc(N3C[C@H](CNC(=O)c4ccc(Cl)s4)OC3=O)ccc21]

200 mg (0.61 mmol) of 6-[(5S)-5-(aminomethyl)-2-oxo-1,3-oxazolidin-3-yl]-3-isopropyl-1,3-benzoxazol-2(33H)-one hydrochloride (EP 738726) are suspended in 5 ml of tetrahydrofuran, and 0.26 ml (1.83 mmol) of triethylamine and 132 mg (0.73 mmol) of 5-chlorothiophene-2-carbonyl chloride are added. The reaction mixture is stirred at room temperature overnight and then concentrated. The product is isolated by column chromatography 4 silica gel, methylene chloride/ethanol=50/l to 20/l). 115 mg (43% of theory) of the desired compound are obtained.

MS (ESI): m/z (%)=436 (M+H, 100);

HPLC (method 4): rt=3.78 min.

The following compounds were prepared in an analogous manner:

| Example No. | Structure | M.p. [° C.] | IC₅₀ [μM] |
|---|---|---|---|
| 48 | 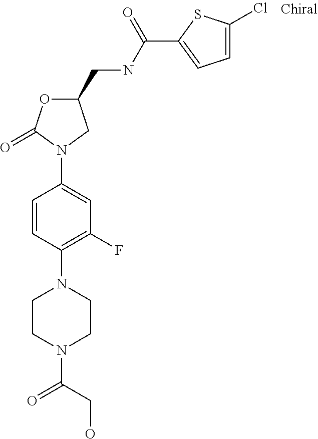 [SMILES: O=C(NC[C@H]1CN(c2ccc(N3CCN(C(=O)CO)CC3)c(F)c2)C(=O)O1)c1ccc(Cl)s1] | 210 | 0.12 |
| 49 | 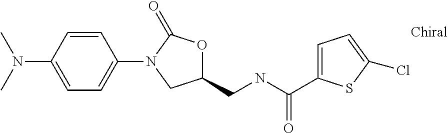 [SMILES: CN(C)c1ccc(N2C[C@H](CNC(=O)c3ccc(Cl)s3)OC2=O)cc1] | 234 | 0.074 |
| 50 | 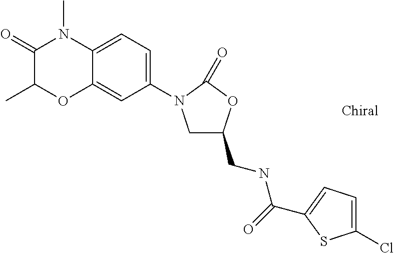 [SMILES: CC1Oc2cc(N3C[C@H](CNC(=O)c4ccc(Cl)s4)OC3=O)ccc2N(C)C1=O] | 195 | 1.15 |
| 51 | 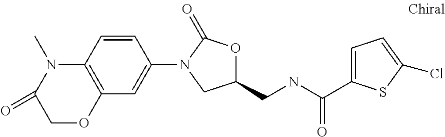 [SMILES: CN1C(=O)COc2cc(N3C[C@H](CNC(=O)c4ccc(Cl)s4)OC3=O)ccc21] | 212 | 1.19 |
| 52 | 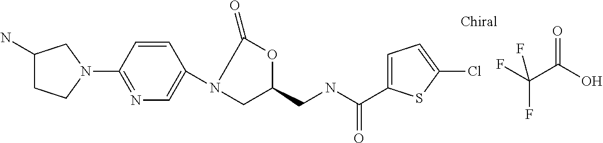 | 160 | 0.19 |
| 53 | 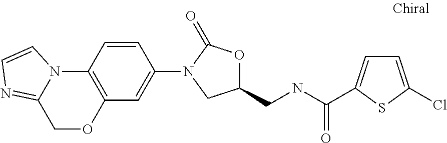 [SMILES: O=C(NC[C@H]1CN(c2ccc3c(c2)OCc2nccn2-3)C(=O)O1)c1ccc(Cl)s1] | MS (ESI): m/z (%) = 431 ([M + H]⁺, 100), Cl pattern | 0.74 |
| 54 | 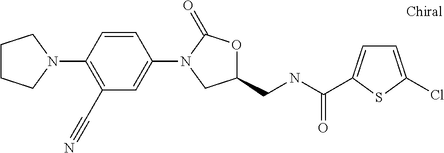 [SMILES: N#Cc1cc(N2C[C@H](CNC(=O)c3ccc(Cl)s3)OC2=O)ccc1N1CCCC1] | 221 | 0.13 |
| | from 5-amino-2- | | |
| | pyrrolidinobenzonitrile (Grell, | | |
| | W., Hurnaus, R.; Griss, G., Sauter, R.; | | |
| | Rupprecht, E. et al.; | | |
| | J. Med. Chem. 1998, 41; 5219 | | |
| 55 | 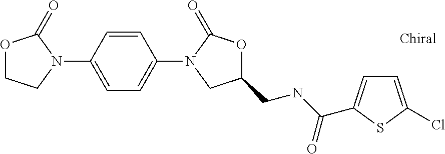 [SMILES: O=C(NC[C@H]1CN(c2ccc(N3CCOC3=O)cc2)C(=O)O1)c1ccc(Cl)s1] | 256 | 0.04 |
| | from 3-(4-aminophenyl)-oxazolidin- | | |
| | 2-one (Artico, M. et al.; Farmaco | | |
| | Ed. Sci. 1969, 24; 179) | | |
| 56 | 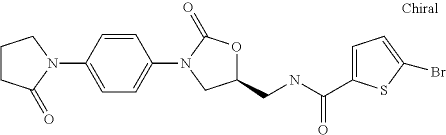 [SMILES: O=C(NC[C@H]1CN(c2ccc(N3CCCC3=O)cc2)C(=O)O1)c1ccc(Br)s1] | 218 | 0.004 |
| 57 | 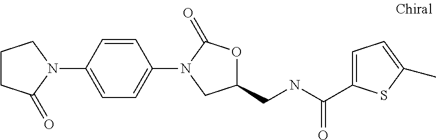 [SMILES: Cc1ccc(C(=O)NC[C@H]2CN(c3ccc(N4CCCC4=O)cc3)C(=O)O2)s1] | 226 | 0.58 |
| 58 | 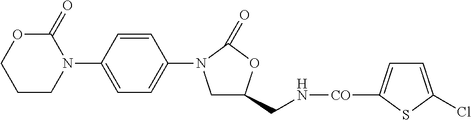 [SMILES: O=C(NC[C@H]1CN(c2ccc(N3CCCOC3=O)cc2)C(=O)O1)c1ccc(Cl)s1] | 228-230 | |

Examples 20 to 30 and 58 to 139 which follow relate to process variant [B], with Examples 20 and 21 describing the preparation of precursors.

### Example 20

### Preparation of N-allyl-5-chloro-2-thiophenecarboxamide

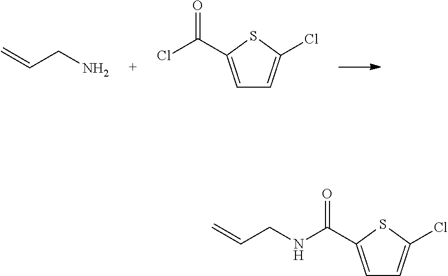

5-Chlorothiophene-2-carbonyl chloride (7.61 g, 42 mmol) is added dropwise to an ice-cooled solution of 2.63 ml (35 mmol) of allylamine in 14.2 ml of absolute pyridine and 14.2 ml of absolute THF. The ice cooling is removed and the mixture is stirred at room temperature for 3 h before being concentrated in vacuo. Water is added to the residue, and the solid is filtered off. The crude product is purified by flash chromatography on silica gel (dichloromethane).

Yield: 7.20 g (99% of theory):

MS (DCI, NH₄): m/z (%)=219 (M+NH₄, 100), 202 (M+H, 32);

HPLC (method 1): rt (%)=3.96 min (98.9).

### Example 21

### Preparation of 5-chloro-N-(2-oxiranylmethyl)-2-thiophenecarboxamide

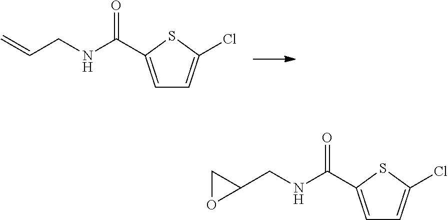

meta-Chloroperbenzoic acid (3.83 g, approx. 60% pure) is added to an ice-cooled solution of 2.0 g (9.92 mmol) of N-allyl-5-chloro-2-thiophenecarboxamide in 10 ml of dichloromethane. The mixture is stirred overnight while warming to room temperature, and then washed with 10% sodium bisulfate solution (three times). The organic phase is washed with saturated sodium bicarbonate solution (twice) and with saturated sodium chloride solution, dried over magnesium sulfate and concentrated. The product is purified by chromatography on silica gel (cyclohexane/ethyl acetate 1:1).

Yield: 837 mg (39% of theory):

MS (DCI, NH₄): m/z (%)=253 (M+NH₄, 100), 218 (M+H, 80);

HPLC (method 1): rt (%)=3.69 min (approx. 80).

General Method for Preparing Substituted N-(3-amino-2-hydroxypropyl)-5-chloro-2-thiophenecarboxamide Derivatives starting from 5-chloro-N-(2-oxiranylmethyl)-2-thiophenecarboxamide

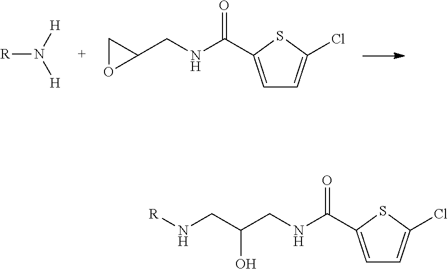

5-Chloro-N-(2-oxiranylmethyl)-2-thiophenecarboxamide (1.0 eq.) is added in portions to a solution of primary amine or aniline derivative (1.5 to 2.5 eq.) in 1,4-dioxane, 1,4-dioxane/water mixtures or ethanol, ethanol/water mixtures (approx. 0.3 to 1.0 mol/l) at room temperature or at temperatures up to 80° C. The mixture is stirred for 2 to 6 hours before being concentrated. The product can be isolated from the reaction mixture by chromatography on silica gel (cyclohexane/ethyl acetate mixtures, dichloromethane/methanol mixtures or dichloromethane/methanol/triethylamine mixtures).

The following were prepared in an analogous manner:

### Example 22

### N-[(3-(Benzylamino)-2-hydroxypropyl]-5-chloro-2-thiophenecarboxamide

MS (ESI): m/z (%)=325 (M+H, 100);

HPLC (method 1): rt (%)=3.87 min (97.9).

### Example 23

### 5-Chloro-N-[(3-(3-cyanoanilino)-2-hydroxypropyl]-2-thiophenecarboxamide

MS (ESI): m/z (%)=336 (M+H, 100):

HPLC (method 2): rt (%)=4.04 min (100).

### Example 24

### 5-Chloro-N-[3-(4-cyanoanilino)-2-hydroxypropyl]-2-thiophenecarboxamide

MS (ESI): m/z (%)=336 (M+H, 100):

HPLC (method 1): rt (%)=4.12 min (100).

### Example 25

### 5-Chloro-N-{3-[4-cyanomethyl)anilino]-2-hydroxypropyl}-2-thiophencarboxamide

MS (ESI): m/z (%)=350 (M+H, 100);

HPLC (method 4): rt (%)=3.60 min (95.4).

### Example 26

### 5-(Chloro-N-{3-[3-(cyanomethyl)anilino]-2-hydroxypropyl}-2-thiophencarboxamide

MS (ESI): m/z (%)=350 (M+H, 100):

HPLC (method 4): rt (%)=3.76 min (94.2).

### Example 58

### tert-Butyl 4-[(3-{[(5-chloro-2-thienyl)carbonyl]amino}-2-hydroxypropyl)amino]-benzylcarbamate

Starting from tert-butyl 4-aminobenzylcarbamate (Bioorg. Med. Chem. Lett.; 1997; 1921-1926):

MS (ES-pos): m/z (%)=440 (M+H, 100), (ES-neg): m/z (%)=438 (M−H, 100);

HPLC (method 1): rt (%)=4.08 (100).

### Example 59

### tert-Butyl 4-[(3-{[(5-chloro-2-thienyl)carbonyl]amino}-2-hydroxypropyl)amino]-phenylcarbamate

Starting from N-tert-butyloxycarbonyl-1,4-phenylenediamine:

MS (ESI): m/z (%)=426 (M+H, 45), 370 (100);

HPLC (method 1): rt (%)=4.06 (100).

### Example 60

### tert-Butyl 2-hydroxy-3-{[4-(2-oxo-1-pyrrolidinyl)phenyl]amino}propylcarbamate

Starting from 1-(4-aminophenyl)-2-pyrrolidinone (Justus Liebigs Ann. Chem.; 1955: 596, 204):

MS (DCI, NH₃): m/z (%)=350 (M+H, 100);

HPLC (method 1): rt (%)=3.57 (97).

### Example 61

### 5-Chloro-N-(3-{[3-fluoro-4-(3-oxo 4-morpholinyl)phenyl]amino}-2-hydroxypropyl)-2-thiophenecarboxamide

800 mg (3.8 mmol) of 4-(4-amino-2-fluoro phenyl)-3-morpholinone and 700 mg (3.22 mmol) of 5-chloro-N-(2-oxiranylmethyl)-2-thiophenecarboxamide are heated in 15 ml of ethanol and 1 ml of water under reflux for 6 hours. The mixture is evaporated in vacuo, the crystals which have separated out after treatment with ethyl acetate are filtered off with suction, and chromatography of the mother liquor results in 276 mg (17% of theory) of the target compound.

R_{f} (ethyl acetate): 0.25.

### Example 62

### (N-(3-Anilino-2-hydroxypropyl)-5-chloro-2-thiophenecarboxamide

starting from aniline:

MS (DCI, NH₃): m/z (%)=311 ([M+H]⁺, 100), Cl pattern;

HPLC (method 3): rt (%)=3.79 (100).

### Example 63

### 5-Chloro-2-hydroxy-3-{[4-(3-oxo-4-morpholinyl)phenyl]amino}propyl)-2-thiophenecarboxamide

starting from 4-(4-aminophenyl)-3-morpholinone:

MS (ESI): m/z (%)=410 ([M+H]⁺, 50), Cl pattern;

HPLC (method 3): rt (%)=3.58 (100).

### Example 64

### N-[3-({4-[Acetyl(cyclopropyl)amino]phenyl}amino)-2-hydroxypropyl]-5-chloro-2-thiophenecarboxamide

starting from N-(4-aminophenyl)-N-cyclopropylacetamide:

MS (ESI): m/z (%)=408 ([M+H]⁺, 100), Cl pattern;

HPLC (method 3): rt (%)=3.77 (100).

### Example 65

### N-[3-({4-[Acetyl(methyl)amino]phenyl}amino)-2-hydroxypropyl]-5-chloro-2-thiophenecarboxamide

starting from N-(4-aminophenyl)-N-methylacetamide:

MS (ESI): m/z (%)=382 (M+H, 100);

HPLC (method 4): rt=3.31 min.

### Example 66

### 5-Chloro-N-(2-hydroxy-3-{[4-(1H-1,2,3-triazol-1-yl)phenyl]amino}propyl)-2-thiophenecarboxamide

starting from 4-4H-1,2,3-triazol-1-yl)aniline (Bouchet et al.; J. Chem. Soc. Perkin Trans. 2; 1974; 449):

MS (ESI): m/z (%)=378 (M+H, 100):

HPLC (method 4): rt=3.55 min.

### Example 67

### Tert-butyl 1-{4-[(3-{[(5-chloro-2-thienyl)carbonyl]amino}-2-hydroxypropyl)amino]phenyl}-L-prolinate

MS (ESI): m/z (%)=480 (M+H, 100);

HPLC (method 4): rt=3.40) min.

### Example 68

### 1-[4-[(3-{[5-Chloro-2-thienyl)carbonyl]amino]-2-hydroxypropyl)amino]phenyl}-4-piperidinecarboxamide

MS (ESI): m/z (%)=437 (M+H, 100);

HPLC (method 4): rt=2.39 min.

### Example 69

### 1-{4-[(3-{[(5-Chloro-2-thienyl)carbonyl]amino}-2-hydroxypropyl)amino]phenyl}-3-piperdinecarboxamide

MS (ESI): m/z (%)=437 (M+H, 100);

HPLC (method 4): rt=2.43 min.

### Example 70

### 5-Chloro-N-(2-hydroxy-3-{[4-(4-oxo-1-piperidinyl)phenyl]amino}propyl)-2-thiophenecarboxamide

MS (ESI): m/z (%)=408 (M+H, 100);

HPLC (method 4): rt=2.43 min.

### Example 71

### 1-{4-[(3-{[(5-Chloro-2-thienyl)carbonyl]amino}-2-hydroxypropyl)amino]phenyl}L-prolinamide

MS (ESI): m/z (%)=423 (M+H, 100);

HPLC (method 4): rt=2.51 min.

### Example 72

### 5-Chloro-N-[2-hydroxy-3-({4-[3-(hydroxymethyl)-1-piperidinyl]phenyl}amino)propyl]-2-thiophenecarboxamide

MS (ESI): m/z (%)=424 (M+H, 100);

HPLC (method 4): rt=2.43 min.

### Example 73

### 5-Chloro-N-[2-hydroxy-3-({4-[2-(hydroxymethyl)-1-piperidinyl]phenyl}amino)propyl]-2-thiophenecarboxamide

MS (ESI): m/z (%)=424 (M+H, 100);

HPLC (method 4): rt=2.49 min.

### Example 74

### Ethyl 1-{4-[(3-{([(5-chloro-2-thienyl)carbonyl]amino}-2-hydroxypropyl)amino]phenyl}-2-piperidincarboxylate

MS (ESI): m/z (%)=466 (M+H, 100);

HPLC (method 4): rt=3.02 min.

### Example 75

### 5-Chloro-N-[2-hydroxy-3-{(4-[2-(hydroxymethyl)-1-pyrrolidinyl]phenyl}amino)propyl]-2-thiophenecarboxamide

MS (ESI): m/z (%)=410 (M+H, 100);

HPLC (method 4): rt=2.48 min.

### Example 76

### 5-Chloro-N-(2-hydroxy-3-{[4-(2-methylhexahydro-5H-pyrrolo[3,4d]isoxazol-5-yl)-phenyl]amino}propyl)-2-thiophenecarboxamide

MS (ESI): m/z (%)=437 (M+H, 100).

HPLC (method 5): rt=1.74 min.

### Example 77

### 5-Chloro-N-(2-hydroxy-3-{[4-(1-pyrrolidinyl)-3-(trifluromethyl)phenyl]amino}propyl)-2-thiophenecarboxamide

MS (ESI): m/z (%)=448 (M+H, 100);

HPLC (method 4): rt=3.30 min.

### Example 78

### 5-Chloro-N-(2-hydroxy-3-{[4-(2-oxo-1-pyrrolidinyl)-3-(trifluoromethyl)phenyl]-amino}propyl)-2-thiophenecarboxamide

MS (ESI): m/z (%)=462 (M+H, 100);

HPLC (method 4): rt=3.50 min.

### Example 79

### 5-Chloro-(3-{[3-chloro-4-(3-oxo-4-morpholinyl)phenyl]amino}-2-hydroxypropyl)-2-thiophenecarboxamide

MS (ESI): m/z (%)=444 (M+H, 100);

HPLC (method 4): rt=3.26 min.

### Example 80

### 5-Chloro-N-(2-hydroxy-3-{[4-(3-oxo-4-morpholinyl-3-trifluoromethyl)phenyl]-amino}propyl)-2-thiophenecarboxamide

MS (ESI): m/z (%)=478 (M+H, 100);

HPLC (method 4): rt=3.37 min.

### Example 81

### 5-Chloro-N-(2-hydroxy-3-{[3-methyl-4-(3-oxo-4-morpholinyl)phenyl]amino}propy)-2-thiophenecarboxamide

MS (ESI): m/z (%)=424 (M+H, 100);

HPLC (method 4): rt=2.86 min.

### Example 82

### 5-Chloro-N-(3-{[-cyano-4-(3-oxo-4-morpholinyl)phenyl]amino}-2-hydroxypropy)-2-thiophenecarboxamide

MS (ESI): m/z (%)=435 (M+H, 100):

HPLC (method 4): rt=3.10 min.

### Example 83

### 5-Chloro-N-(3-{[3-chloro-(1-pyrrolidinyl)phenyl]amino}-2-hydroxypropy)-2-thiophenecarboxamide

MS (ESI): m/z (%)=414 (M+H, 100);

HPLC (method 4): rt=2.49 min.

### Example 84

### 5-Chloro-N-(3-{[3-chloro-4-(2-oxo-1-pyrrolidinyl)phenyl]amino}-2-hydroxypropy)-2-thiophenecarboxamide

MS (ESI): m/z (%)=428 (M+H, 100);

HPLC (method 4): rt=3.39 min.

### Example 85

### 5-Chloro-N-(3-{[3,5-dimethyl-4-(3-oxo-4-morpholinyl)phenyl]amino}-2-hydroxypropyl)-2-thiophenecarboxamide

MS (ESI): m/z (%)=438 (M+H, 100);

HPLC (method 4): rt=2.84 min.

### Example 86

### N-(3-{[3-(Aminocarbonyl)-4-(4-morpholinyl)phenyl]amino}-2-hydroxypropy)-5-chloro-2-thiophenecarboxamide

MS (ESI): m/z (%)=439 (M+H, 100);

HPLC (method 4): rt=2.32 min.

### Example 87

### 5-Chloro-N-(2-hydroxy-3-{[3-methoxy-4-(4-morpholinyl)phenyl]amino}propyl)-2-thiophenecarboxamide

MS (ESI): m/z (%)=426 (M+H, 100);

HPLC (method 4): rt=2.32 min.

### Example 88

### N-(3-{[3-Acetyl-4-(4-morpholinyl)phenyl]amino}-2-hydroxypropyl)-5-chloro-2-thiophenecarboxamide

MS (ESI): m/z (%)=438 (M+H, 100);

HPLC (method 4): rt=2.46 min.

### Example 89

### N-(3-{[3-Amino-4-(3-oxo-4-morpholinyl)phenyl]amino}-2-hydroxypropy)-5-chloro-2-thiophenecarboxamide

MS (ESI): m/z (%)=425 (M+H, 100);

HPLC (method 4): rt=2.45 min.

### Example 90

### 5-Chloro-N-(3-{[3-chloro-4-(2-methyl-3-oxo-4-morpholinyl)phenyl]amino}-2-hydroxypropyl)-2-thiophenecarboxamide

MS (ESI): m/z (%)=458 (M+H, 100);

HPLC (method 4): rt=3.44 min.

### Example 91

### 5-Chloro-N-(3-{[3-chloro-4-(2-methyl-5-exo-4-morpholinyl)phenyl]amino}-2-hydroxypropyl)-2-thiophenecarboxamide

MS (ESI): m/z (%)=458 (M+H, 100);

HPLC (method 4): rt=3.48 min.

### Example 91a

### 5-Chloro-N-[2-hydroxy-3-({4-[(3-oxo-4-morpholinyl)methyl]phenyl}amino)propyl]-2-thiophenecarboxamide

Starting from 4-(4-aminobenzyl)-3-morpholinone (Surrey et al.; J. Amer. Chem. Soc.; 77; 1955; 633):

MS (ESI): m/z (%)=424 (M+H, 100):

HPLC (method 4): rt=2.66 min.

### General Method for Preparing 3-substituted 5-chloro-N-[(2-oxo-1,3-oxazolidin-5-yl)methyl]-2-thiophenecarboxamide Derivatives Starting from Substituted N-(3-amino-2-hydroxypropyl)-5-chloro-2-thiophenecarboxamide Derivatives

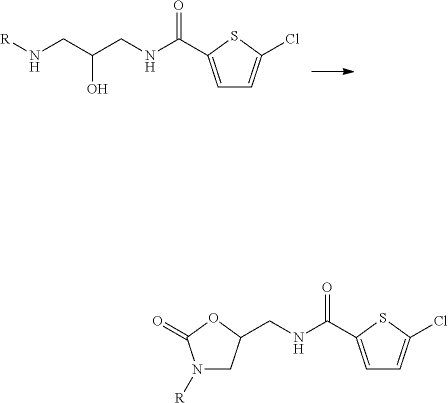

Carbodiimidazole (1.2 to 1.8 eq.) or a comparable phosgene equivalent is added to a solution of substituted N-(3-amino-2-hydroxypropyl)-5-chloro-2-thiophenecarboxamide derivative (1.0 eq.) in absolute THF (approx. 0.1 mol/l) at room temperature. The mixture is stirred at room temperature or, where appropriate, at elevated temperature (up to 70° C.) for 2 to 18 h before being concentrated in vacuo. The product can be purified by chromatography on silica gel (dichloromethane/methanol mixtures or cyclohexane/ethyl acetate mixtures).

The following were prepared in an analogous manner:

### Example 27

### N-[(3-Benzyl-2-oxo-1,3-oxazolidin-5-yl)methyl]-5-chloro-2-thiophenecarboxamide

MS (DCI, NH₄): m/z (%)=372 (M+Na, 100), 351 (M+H, 45);

HPLC (method 1): rt (%)=4.33 min (100).

### Example 28

### 5-Chloro-N-{[3-(3-cyanophenyl)-2-oxo-1,3-oxazolidin-5-yl]methyl}-2-thiophenecarboxamide

MS (DCI, NH₄): m/z (%)=362 (M+H, 42), 145 (100);

HPLC (method 2): rt (%)=4.13 min (100).

### Example 29

### 5-Chloro-N-({3-[4-(cyanomethyl)phenyl]-2-oxo-1,3-oxazolidin-5-yl}methyl)-2-thiophenecarboxamide

MS (ESI): m/z (%)=376 (M+H, 100):

HPLC (method 4): rt=4.12 min

### Example 30

### 5-Chloro-N-({3-[3-(cyanomethyl)phenyl]-2-oxo-1,3-oxazolidin-5-yl}methyl)-2-thiophenecarboxamide

MS (ESI): m/z (%)=376 (M+H, 100);

HPLC (method 4): rt=4.17 min

### Example 92

### tert-Butyl 4-[5-({[(5-chloro-2-thienyl)carbonyl]amino}methyl)-2-oxo-1,3-oxazolidin-3-yl]benzylcarbamate

starting from Example 58:

MS (ESI): m/z (%)=488 (M+Na, 23), 349 (100);

HPLC (method 1): rt (%)=4.51 (98.5).

### Example 93

### tert-Butyl 4-[5-({[(5-chloro-2-thienyl)carbonyl]amino}methyl)-2-oxo-1,3-oxazolidin-3-yl]phenylcarbamate

starting from Example 59:

MS (ESI): m/z (%)=493 (M+Na, 70), 452 (M+H, 10), 395 (100);

HPLC (method 1): rt (%)=4.41 (100).

### Example 94

### tert-Butyl 2-oxo-3-[4-(2-oxo-1-pyrrolidinyl)phenyl]-3-oxazolidin-5-yl}methylcarbamate

starting from Example 60:

MS (DCI, NH₃): m/z (%)=393 (M+NH₄, 100);

HPLC (method 3): rt (%)=3.97 (100).

### Example 95

### 5-Chloro-N-({3-[3-fluoro-4-(3-oxo-4-morpholinyl)phenyl]-2-oxo-1,3-oxazolidin-5-yl}methyl)-2-thiophenecarboxamide

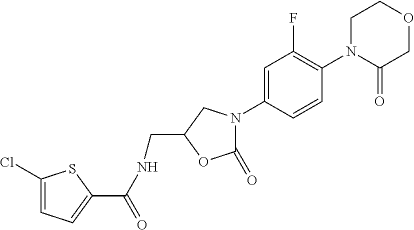 [SMILES: O=C(NCC1CN(c2ccc(N3CCOCC3=O)c(F)c2)C(=O)O1)c1ccc(Cl)s1]

260 ng (0.608 mmol) of 5-chloro-N-(3-{([3-fluoro-4-(3-oxo-4-morpholinyl)phenyl]amino}-2-hydroxypropyl)-2-thiophenecarboxamide (from Example 61), 197 mg (1.22 mmol) of carbonylimidazole and 7 mg of dimethylaminopyridine are boiled in 20 ml of dioxane under reflux for 5 hours. 20 ml of acetonitrile are then added, and the mixture is stirred in a close container in a microwave oven at 180° C. for 30 minutes. The solution is concentrated in a rotary evaporator and chromatographed on an RP-HPLC column. 53 mg (19% of theory) of the target compound are obtained.

NMR (300 MHz, d₆-DMSO): δ=3.6-3.7 (m, 4H), 3.85 (dd, 1H), 3.95 (m, 2H), 4.2 (m, 1H), 4.21 (s, 2H), 4.85 (m, 1H), 4.18 (s, 2H), 7.19 (d, 1H, thiophene), 7.35 (dd, 1H), 7.45 (t, 1H), 7.55 (dd, 1H), 7.67 (d, 1H, thiophene), 8.95 (t, 1H, CONH).

### Example 96

### 5-Chloro-N-[(2-oxo-3-phenyl-1,3-oxazolidin-5-yl)methyl]-2-thiophenecarboxamide

starting from Example 62:

MS (ESI): m/z (%)=359 ([M+Na]⁺, 71), 337 ([M+H]⁺, 100), Cl pattern;

HPLC (method 3): rt (%)=4.39 (100).

IC₅₀: 2 μM

### Example 97

### 5-Chloro-N-({2-oxo-3-[4-(3-oxo-4-morpholinyl)phenyl]-1,3-oxazolidin-5-yl}methyl)-2-thiophenecarboxamide

starting from Example 63:

MS (ESI): m/z (%)=458 ([M+Na]⁺, 66), 436 ([M+H]⁺, 100), Cl pattern;

HPLC (method 3): rt (%)=3.89 (100).

IC₅₀: 1.4 nM

### Example 98

### N-[(3-{4-[Acetyl(cyclopropyl)amino]phenyl}-2-oxo-1,3-oxazolidin-5-yl)methyl]-5-chloro-2-thiophenecarboxamide

starting from Example 64:

MS (ESI): m/z (%)=456 ([M+Na]⁺, 55), 434 ([M+H]⁺, 100), Cl pattern;

HPLC (method 3): rt (%)=4.05 (100).

IC₅₀: 50 nM

### Example 99

### N-[(3-{4-[Acetyl(methyl)amino]phenyl}-2-oxo-1,3-oxazolidin-5-yl)methyl]-5-cloro-2-thiophenecarboxamide

MS (ESI): m/z (%)=408 (M+H, 30), 449 (M+H+M+MeCN, 100);

HPLC (method 4): rt=3.66 min.

### Example 100

### 5-Chloro-N-({2-oxo-3-[4-(1H-1,2,3-triazol-1-yl)phenyl]-1,3-oxazolidin-5-yl}methyl)-2-thiophenecarboxamide

MS (ESI): m/z (%)=404 (M+H, 45), 445 (M+H+MeCN, 100);

HPLC (method 4): rt=3.77 min.

### Example 101

### Tert-butyl 1-{4-[5-({[(5-chloro-2-thienyl)carbonyl]amino}methyl)-2-oxo-1,3-oxazolidin-3-yl]phenyl}-L-prolinate

MS (ESI): m/z (%)=450 (M+H-56, 25), 506 (M+H, 100);

HPLC (method 4): rt=5.13 min.

### Example 102

### 1-{4-[5-({[(5-Chloro-2-thienyl)carbonyl]amino}methy)-2-oxo-1,3-oxazolidin-3-yl]phenyl}-4-piperidinecarboxamide

MS (ESI): m/z (%)=463 (M+H, 100);

HPLC (method 4): rt=2.51 min.

### Example 103

### 1-{4-[5-({[5-Chloro-2-thienyl)carbonyl]amino}methyl)-2-oxo-1,3-oxazolidin-3-yl]phenyl}-3-piperidinecarboxamide

MS (ESI): m/z (%)=463 (M+H, 100);

HPLC (method 4): rt=2.67 min.

### Example 104

### 5-Chloro-N-({2-oxo-3-[4-(4-oxo-1-piperidinyl)phenyl]-1,3-oxazolidin-5-yl}methyl)-2-thiophenecarboxamide

MS (ESI): m/z (%)=434 (M+H, 40), 452 (M+H+H₂O, 100), 475 (M+H+MeCN, 60);

HPLC (method 4): rt=3.44 min.

### Example 105

### 1-{4-[5-({[5-Chloro-2-thienyl)carbonyl]amino}methyl)-2-oxo-1,3-oxazolidin-3-yl]phenyl}-L-prolinamide

MS (ESI): m/z (%)=449 (M+H, 100);

HPLC (method 4): rt=3.54 min.

### Example 106

### 5-Chloro-N-[(3-{4-[3-(hydroxymethyl)-1-piperidinyl]phenyl}-2-oxo-1,3-oxazolidin-5-yl)methyl]-2-thiophenecarboxamide

MS (ESI): m/z (%)=450 (M+H, 100);

HPLC (method 5): rt=2.53 min.

### Example 107

### 5-Chloro-N-[(3-{4-[2-(hydroxymethyl)-1-piperidinyl]phenyl}-2-oxo-1,3-oxazolidin-5-yl)methyl]-2-thiophenecarboxamide

MS (ESI): m/z (%)=450 (M+H, 100):

HPLC (method 5): rt=2.32 min.

### Example 108

### Ethyl 1-{4[5-({[(5-chloro-2-thienyl)carbonyl]amino}methyl)-2-oxo-1,3-oxazolin-3-yl]phenyl}-2-piperidinecarboxylate

MS (ESI): m/z (%)=492 (M+H, 100);

HPLC (method 5): rt=4.35 min.

### Example 109

### 5-Chloro-N-[(3-{4-[2-(hydroxymethyl)-1-piperidinyl]phenyl}-2-oxo-1,3-oxazolidin-5-yl)methyl]-2-thiophenecarboxamide

MS (ESI): m/z (%)=436 (M+H, 100);

HPLC (method 4): rt=2.98 min.

### Example 110

### 5-Chloro-N-({2-oxo-3-[4-(1-pyrrolidinyl)-3-(trifluoromethyl)phenyl]-1,3-oxazolidin-5-yl}methyl)-2-thiophenecarboxamide

MS (ESI): m/z (%)=474 (M+H, 100);

HPLC (method 4): rt=4.63 min.

### Example 111

### 5-Chloro-N-({3-[4-(2-methylhexahydro-5H-pyrrolo[3,4-d]isoxazol-5-yl)phenyl]-2-oxo-1,3-oxazolidin-5-yl}methyl)-2-thiophenecarboxamide

MS (ESI): m/z (%)=463 (M+H, 100);

HPLC (method 4): rt=2.56 min.

### Example 112

### 5-Chloro-N-({2-oxo-3-[4-(2-oxo-1-pyrrodinyl)-3-(trifluoromethyl)phenyl]1,3-oxazolidin-5-yl}methyl)-2-thiophenecarboxamide

MS (ESI): m/z (%)=488 (M+H, 100);

HPLC (method 4): rt=3.64 min.

### Example 113

### 5-Chloro-N-({3-[3-chloro-4-(3-oxo-4-morpholinyl)phenyl]-2-oxo-1,3-oxazolidin-5-yl}methyl)-2-thiophenecarboxamide

MS (ESI): m/z (%)=470 (M+H, 100);

HPLC (method 4): rt=3.41 min.

### Example 114

### 5-Chloro-N-({2-oxo-3-[4-(3-oxo-4-morpholinyl)-3-(trifluoromethyl)phenyl]-1,3-oxazolidin-5-yl}methyl)-2-thiophenecarboxamide

MS (ESI): m/z (%)=504 (M+H, 100);

HPLC (method 4): rt=3.55 min.

### Example 115

### 5-Chloro-N-({3-[3-methyl-4-(3-oxo-4-morpholinyl)phenyl]-2-oxo-1,3-oxazolidin-5-yl}methyl)-2-thiophenecarboxamide

MS (ESI): m/z (%)=450 (M+H, 100);

HPLC (method 4): rt=3.231 min.

### Example 116

### 5-Chloro-N-({3-[3-cyano-4-(3-oxo-4-morpholinyl)phenyl]-2-oxo-1,3-oxazolidin-5-yl}methyl)-2-thiophenecarboxamide

MS (ESI): m/z (%)=461 (M+H, 100);

HPLC (method 4): rt=3.27 min.

### Example 117

### 5-Chloro-N-({3-[3-chloro-4-(1-pyrrolidinyl)phenyl]-2-oxo-1,3-oxazolidin-5-yl}methyl)-2-thiophenecarboxamide

MS (ESI): m/z (%)=440 (M+H, 100);

HPLC (method 4): rt=3.72 min.

### Example 118

### 5-Chloro-N-({3-[3-chloro-4-(2-oxo-1-pyrrolidinyl)phenyl]-2-oxo-1,3-oxazolidin-5-yl}methyl)-2-thiophenecarboxamide

MS (ESI): m/z (%)=454 (M+H, 100);

HPLC (method 4): rt=3.49 min.

### Example 119

### 5-Chloro-N-({3-[3,5-dimethyl-4-(3-oxo-4-morpholinyl)phenyl]-2-oxo-1,3-oxazolidin-5-yl}methyl)-2-thiophenecarboxamide

MS (ESI): m/z (%)=464 (M+H, 100);

HPLC (method 4): rt=3.39 min.

### Example 120

### N-({3-[3-(Aminocarbonyl)-4-(4-morpholinyl)phenyl]-2-oxo-1,3-oxazolidin-5-yl}methyl)-5-chloro-2-thiophenecarboxamide

MS (ESI): m/z (%)=464 (M+H, 100);

HPLC (method 4): rt=3.07 min.

### Example 121

### 5-Chloro-N-({3-[3-methoxy-4-(4-morpholinyl)phenyl]-2-oxo-1,3-oxazolidin-5-yl}methyl)-2-thiophenecarboxamide

MS (ESI): m/z (%)=452 (M+H, 100);

HPLC (method 4): rt=2.86 min.

### Example 122

### N-({3-[3-Acetyl-4-(4-morpholinyl)phenyl]-2-oxo-1,3-oxazolidin-5-yl}methyl)-5-chloro-2-thiophenecarboxamide

MS (ESI): m/z (%)=464 (M+H, 100);

HPLC (method 4): rt=3.52 min.

### Example 123

### N-({3-[3-Amino-4-(3-oxo-4-morpholinyl)phenyl]-2-oxo-1,3-oxazolidin-5-yl}methyl)-5-chloro-2-thiophenecarboxamide

MS (ESI) m/z (%)=451 (M+H, 100);

HPLC (method 6): rt=3.16 min.

### Example 124

### 5-Chloro-N-({3-[3-chloro-4-(2-methyl-3-oxo-4-morpholinyl)phenyl]-2-oxo-1,3-oxazolidin-5-yl}methyl)-2-thiophenecarboxamide

MS (ESI): m/z (%)=484 (M+H, 100);

HPLC (method 4): rt=3.59 min.

### Example 125

### 5-Chloro-N-({3-[3-chloro-4-(2-methyl-5-oxo-4-morpholinyl)phenyl]-2-oxo-1,3-oxazolidin-5-yl}methyl)-2-thiophenecarboxamide

MS (ESI): m/z (%)=484 (M+H, 100);

HPLC (method 4): rt=3.63 min.

### Example 125a

### 5-Chloro-N-[(2-oxo-3-{4-[(3-oxo-4-morpholinyl)methyl]phenyl}-1,3-oxazolidin-5-yl)methyl]-2-thiophenecarboxamide

MS (ESI): m/z (%)=450 (M+H, 100);

HPLC (method 4): rt=3.25 min.

In addition, the following compounds were prepared by the route of epoxide opening with an amine and subsequent cyclization to give the corresponding oxazolidinone:

| Example No. | Structure | M.p. [° C.] | IC₅₀ [μM] |
|---|---|---|---|
| 126 | 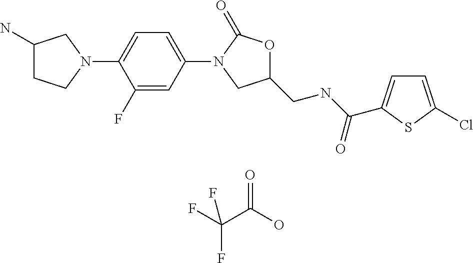 [SMILES: NC1CCN(c2ccc(N3CC(CNC(=O)c4ccc(Cl)s4)OC3=O)cc2F)C1 ; O=C(O)C(F)(F)F] | 229 decomp. | 0.013 |
| 127 | 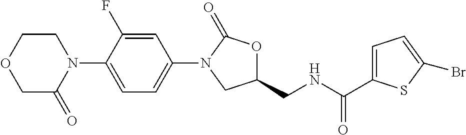 [SMILES: O=C(NC[C@H]1CN(c2ccc(N3CCOCC3=O)c(F)c2)C(=O)O1)c1ccc(Br)s1] | 159 | 0.0007 |
| 128 | 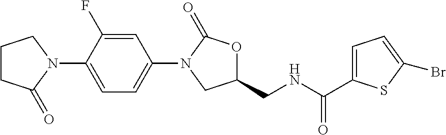 [SMILES: O=C(NC[C@H]1CN(c2ccc(N3CCCC3=O)c(F)c2)C(=O)O1)c1ccc(Br)s1] | 198 | 0.002 |
| 129 | 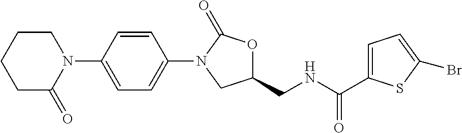 [SMILES: O=C(NC[C@H]1CN(c2ccc(N3CCCCC3=O)cc2)C(=O)O1)c1ccc(Br)s1] | 196 | 0.001 |
| 130 | 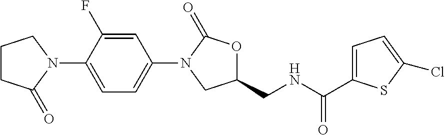 [SMILES: O=C(NC[C@H]1CN(c2ccc(N3CCCC3=O)c(F)c2)C(=O)O1)c1ccc(Cl)s1] | 206 | 0.0033 |
| 130a | 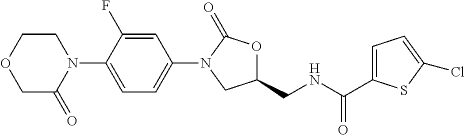 [SMILES: O=C(NC[C@H]1CN(c2ccc(N3CCOCC3=O)c(F)c2)C(=O)O1)c1ccc(Cl)s1] | 194 | |
| 131 | 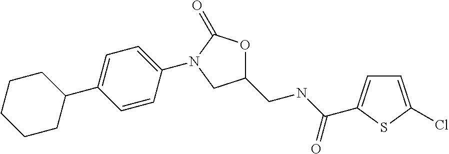 [SMILES: O=C(NCC1CN(c2ccc(C3CCCCC3)cc2)C(=O)O1)c1ccc(Cl)s1] | 195 | 0.85 |
| 132 | 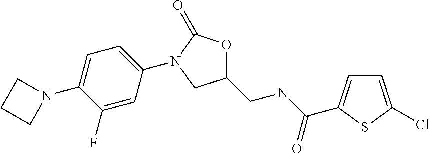 [SMILES: O=C(NCC1CN(c2ccc(N3CCC3)c(F)c2)C(=O)O1)c1ccc(Cl)s1] | 206 | 0.12 |
| 133 | 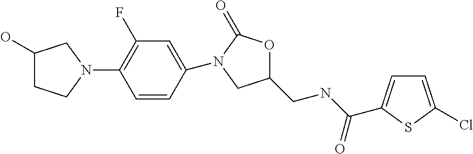 [SMILES: O=C(NCC1CN(c2ccc(N3CCC(O)C3)c(F)c2)C(=O)O1)c1ccc(Cl)s1] | 217 | 0.062 |
| 134 | 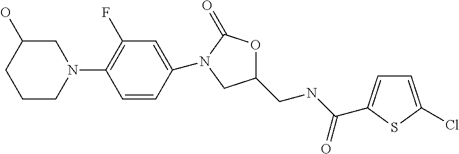 [SMILES: O=C(NCC1CN(c2ccc(N3CCCC(O)C3)c(F)c2)C(=O)O1)c1ccc(Cl)s1] | 207 | 0.48 |
| | from 1-(4-amino-phenyl)piperidin-3- | | |
| | ol (Tong, L.K.J. et al; | | |
| | J. Amer. Chem. Soc 1960; 82, 1988). | | |
| 135 | 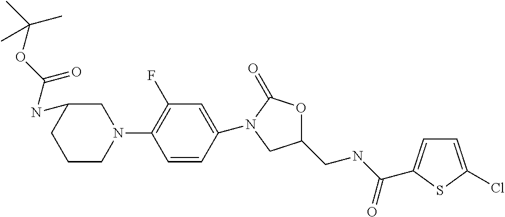 [SMILES: CC(C)(C)OC(=O)NC1CCCN(c2ccc(N3CC(CNC(=O)c4ccc(Cl)s4)OC3=O)cc2F)C1] | 202 | 1.1 |
| 136 | 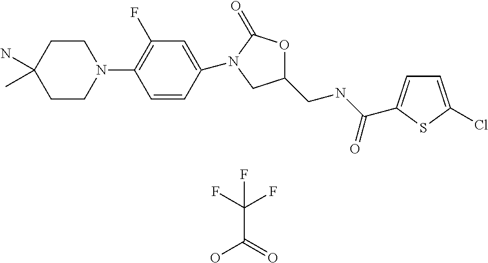 [SMILES: CC1(N)CCN(c2ccc(N3CC(CNC(=O)c4ccc(Cl)s4)OC3=O)cc2F)CC1 ; O=C(O)C(F)(F)F] | 239 | 1.2 |
| 137 | 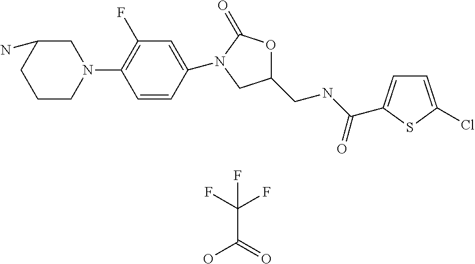 [SMILES: NC1CCCN(c2ccc(N3CC(CNC(=O)c4ccc(Cl)s4)OC3=O)cc2F)C1 ; O=C(O)C(F)(F)F] | 219 | 0.044 |
| 138 | 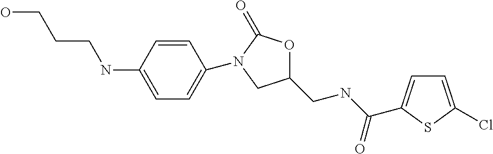 [SMILES: O=C(NCC1CN(c2ccc(NCCCO)cc2)C(=O)O1)c1ccc(Cl)s1] | 95 | 0.42 |
| 139 | 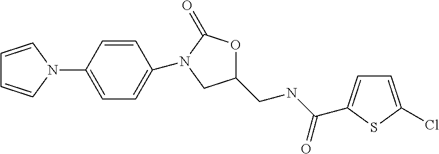 [SMILES: O=C(NCC1CN(c2ccc(-n3cccc3)cc2)C(=O)O1)c1ccc(Cl)s1] | 217 | 1.7 |

Examples 14 to 16 which follow are exemplary embodiments of the optional oxidation process step, i.e. one which takes place where appropriate.

### Example 14

### 5-Chloro-N-({5S)-3-[3-fluoro-4-(1-oxo-1[lambda]⁴,4-thiazinan-4-yl)phenyl]-2-oxo-1,3-oxazolidin-5-yl}methyl)-2-thiophenecarboxamide

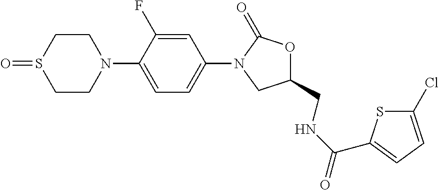 [SMILES: O=C(NC[C@H]1CN(c2ccc(N3CCS(=O)CC3)c(F)c2)C(=O)O1)c1ccc(Cl)s1]

5-Chlor-N-({(5S)-3-[3-fluoro-4-(1,4-thiazinan-4-yl}phenyl]-2-oxo-1,3-oxazolidin-5-yl)methyl)-2-thiophenecarboxamide (0.1 g, 0.22 mmol) from Example 3 in methanol (0.77 ml) is added at 0° C. to a solution of sodium periodate (0.05 g, 0.23 mmol) in water (0.54 ml) and stirred at 0° C. for 3 h. Then 1 ml of DMF is added, and the mixture is stirred at RT for 8 h. Addition of a further 50 mg of sodium periodate is followed by stirring at RT once again overnight. 50 ml of water are then added to the mixture, and the insoluble product is filtered off with suction. Washing with water and drying result in 60 mg (58% of theory) of crystals.

M.p.: 257° C.;

R_{f} (silica gel, toluene/ethyl acetate 1:1)=0.54 (precursor=0.46);

IC₅₀=1.1 μM;

MS (DCI) 489 (M+NH₄), Cl pattern.

### Example 15

### Preparation of 5-chloro-N-({(5S)-3-[4-(1,1-dioxo-1[lambda]⁶,4-thiazinan-4-yl)-3-fluorophenyl]-2-oxo-1,3-oxazolidin-5-yl}methyl)-2-thiophenecarboxamide

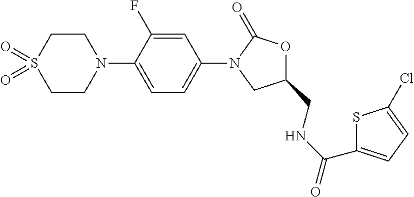 [SMILES: O=C(NC[C@H]1CN(c2ccc(N3CCS(=O)(=O)CC3)c(F)c2)C(=O)O1)c1ccc(Cl)s1]

80 mg (0.66 mmol) of N-methylmorpholine N-oxide (NMO) and 0.1 ml of a 2.5% strength solution of osmium tetroxide in 2-methyl-2-propanol are added to 5-chloro-N-({(5S)-3-[3-fluoro-4-(1,4-thiazinan-4-yl)phenyl]-2-oxo-1,3-oxazolidin-5-yl}methyl)-2-thiophenecarboxamide from Example 3 (0.1 g, 0.22 mmol) in 3.32 ml of a mixture of 1 part of water and 3 parts of acetone. The mixture is stirred at room temperature overnight and a further 40 mg of NMO are added. After being stirred for a further night, the mixture is added to 50 ml of water and extracted three times with ethyl acetate. Drying and evaporation of the organic phase result in 23 mg, and filtration with suction of the insoluble solid from the aqueous phase results in 19 mg of the target compound (total 39% of theory).

M.p.: 238° C.;

R_{f}(toluene/ethyl acetate 1:1)=0.14 (precursor=0.46);

IC₅₀=210 nM;

MS (DCI): 505 (M+NH₄), Cl pattern.

### Example 16

### 5-Chloro-N-{[(5S)-3-(3-fluoro-4-morpholinophenyl)-2-oxo-1,3-oxazolidin-5-yl]methyl}-2-thiophenecarboxamide N-oxide

is obtained by treating 5-chloro-N-{[(5S)-3-(3-fluoro-4-morpholinophenyl)-2-oxo-1,3-oxazolidin-5-yl]methyl}-2-thiophenecarboxamide from Example 1 with monoperoxyphthalic acid magnesium salt.

MS (ESI): 456 (M+H, 21%, Cl pattern), 439 (100%).

Examples 31 to 35 and 140 to 147 which follow relate to the optional amidination process step, i.e. one which takes place where appropriate.

General Method for Preparing Amidines and Amidine Derivatives Starting from cyanomethylphenyl-substituted 5-chloro-N-[(2-oxo-1,3-oxazolidin-5-yl)methyl]-2-thiophenecarboxamide Derivatives

The particular cyanomethylphenyl-substituted 5-chloro-N-[(2-oxo-1,3-oxazolidin-5-yl)methyl]-2-thiophenecarboxamide derivate (1.0 eq.) is stirred together with triethylamine (8.0 eq.) in a saturated solution of hydrogen sulfide in pyridine (approx. 0.05-0.1 mol/l) at RT for one to two days. The reaction mixture is diluted with ethyl acetate (EtOAc) and washed with 2 N hydrochloric acid. The organic phase is dried with MgSO₄, filtered and evaporated in vacuo.

The crude product is dissolved in acetone (0.01-0.1 mol/l), and methyl iodide (40 eq.) is added. The reaction mixture is stirred at room temperature (RT) for 2 to 5 h and then concentrated in vacuo.

The residue is dissolved in methanol (0.01-0.1 mol/l) and, to prepare the unsubstituted amidines, ammonium acetate (3 eq.) and ammonium chloride (2 eq.) are added. The substituted amidine derivatives are prepared by adding primary or secondary amines (1.5 eq.) and acetic acid (2 eq.) to the methanolic solution. After 5-30 h, the solvent is removed in vacuo and the residue is purified by chromatography on an RP8 silica gel column (water/acetonitrile 9/1-1/1+0.1% trifluoroacetic acid).

The following were prepared in an analogous manner:

### Example 31

### N-({3-[4-(2-Amino-2-iminoethyl)phenyl]-2-oxo-1,3-oxazolidin-5-yl}methyl)-5-chloro-2-thiophenecarboxamide

MS (ESI): m/z (%)=393 (M+H, 100));

HPLC (method 4): rt=2.63 min

### Example 32

### 5-Chloro-N-({3-[3-(4,5-dihydro-1H-imidazol-2-ylmethyl)phenyl]-2-oxo-1,3-oxazolidin-5-yl}methyl)-2-thiophenecarboxamide

MS (ESI): m/z (%)=419 (M+H, 100);

HPLC (method 4): rt=2.61 min

### Example 33

### 5-Chloro-N-[(3-{3-[2-imino-2-(4-morpholinyl)ethyl]phenyl}-2-oxo-1,3-oxazolidin-5-yl)methyl]-2-thiophenecarboxamide

MS (ESI): m/z (%)=463 (M+H, 100);

HPLC (method 4): rt=2.70 min

### Example 34

### 5-Chloro-N-[(3-{3-[2-imino-2-(1-pyrrolidinyl)ethyl]phenyl}-2-oxo-1,3-oxazolidin-5-yl)methyl]-2-thiophenecarboxamide

MS (ESI): m/z (%)=447 (M+H, 100):

HPLC (method 4): rt=2.82 min

### Example 35

### N-({3-[3-(2-Amino-2-iminoethyl)phenyl]-2-oxo-1,3-oxazolidin-5-yl}methyl)-5-chloro-2-thiophenecarboxamide

MS (ESI): m/z (%)=393 (M+H, 100);

HPLC (method 4): rt=2.60 min

### Example 140

### 5-Chloro-N-({3-[4-(4,5-dihydro-1H-imidazol-2-ylmethyl)phenyl]-2-oxo-1,3-oxazolidin-5-yl}methyl)-2-thiophenecarboxamide

MS (ESI): m/z (%)=419 (M+H, 100);

HPLC (method 4): rt=2.65 min

### Example 141

### 5-Chloro-N-[(3-{4-[2-imino-2-(4-morpholinyl)ethyl]phenyl}-2-oxo-1,3-oxazolidin-5-yl)methyl]-2-thiophenecarboxamide

MS (ESI): m/z (%)=463 (M+H, 100);

HPLC (method 4): rt=2.65 min

### Example 142

### 5-Chloro-N-[(3-{4-[2-imino-2-(1-piperidinyl)ethyl]phenyl}-2-oxo-1,3-oxazolidin-5-yl)methyl]-2-thiophenecarboxamide

MS (ESI): m/z (%)=461 (M+H, 100);

HPLC (method 4): rt=2.83 min

### Example 143

### 5-Chloro-N-[(3-{4-[2-imino-2-(1-pyrrolidinyl)ethyl]phenyl}-2-oxo-1,3-oxazolidin-5-yl)methyl]-2-thiophencarboxamide

MS (ESI): m/z (%)=447 (M+H, 100);

HPLC (method 4): rt=2.76 min

### Example 144

### 5-Chloro-N-[3-{4-[2-cyclopentylamino)-2-iminoethyl]phenyl}-2-oxo-1,3-oxazolidin-5-yl)methyl]-2-thiophenecarboxamide

MS (ESI): m/z (%)=461 (M+H, 100);

HPLC (method 4): rt=2.89 min

### Example 145

### 5-Chloro-N-{[3-(4-{2-imino-2-[(2,2,2-trifluoroethyl)amino]ethyl}phenyl)-2-oxo-1,3-oxazolidin-5-yl]methyl}-2-thiophenecarboxamide

MS (ESI): m/z (%)=475 (M+H, 100);

HPLC (method 4): rt=2.79 min

### Example 146

### N-({3-[4-(2-Amino-2-iminoethyl)phenyl]-2-oxo-1,3-oxazolidin-5-yl}methyl)-5-chloro-2-thiophenecarboxamide

MS (ESI): m/z (%)=469 (M+H, 100);

HPLC (method 4): rt=2.83 min

### Example 147

### 5-Chloro-N-[(3-{4-[2-imino-2-(2-pyridinylamino)ethyl]phenyl}-2-oxo-1,3-oxazolidin-5-yl)methyl]-2-thiophenecarboxamide

MS (ESI): m/z (%)=470 (M+H, 100);

HPLC (method 4): rt=2.84 min

Examples 148 to 151 which follow relate to the elimination of BOC amino protective groups:

General Method for Eliminating Boc Protective Groups (Tert-Butyloxycarbonyl):

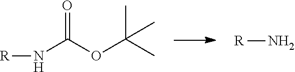

Aqueous trifluoroacetic acid (TPA, approx. 90%) is added dropwise to an ice-cooled solution of a tert-butyloxycarbonyl-(Boc)-protected compound in chloroform or dichloromethane (approx. 0.1 to 0.3 mol/l). After about 15 min, the ice cooling is removed and the mixture is stirred at room temperature for about 2-3 h before the solution is concentrated and dried under high vacuum. The residue is taken up in dichloromethane or dichloromethane/methanol and washed with saturated sodium bicarbonate or 1N sodium hydroxide solution. The organic phase is washed with saturated sodium chloride solution, dried over a little magnesium sulfate and concentrated. Purification takes place where appropriate by crystallization from ether or ether/dichloromethane mixtures.

The following were prepared in an analogous manner from the appropriate Boc-protected precursors:

### Example 148

### N-({3-[4-(Aminomethyl)phenyl]-2-oxo-1,3-oxazolidin-5-yl}methyl)-5-chloro-2-thiophenecarboxamide

starting from Example 92:

MS (ESI): m/z (% k)=349 (M-NH₂, 25), 305 (100);

HPLC (method 1): rt (%)=3.68 (98).

IC₅₀: 2.2 μM

### Example 149

### N-{[3-(4-Aminophenyl)-2-oxo-1,3-oxazolidin-5-yl]methyl}-5-chloro-2-thiophencarboxamide starting from Example 93

MS (ESI): m/z (%)=352 (M+H, 25);

HPLC (method 1): rt (%)=3.50 (100).

IC₅₀: 2 μM

An enantiopure alternative synthesis of this compound is depicted in the following scheme (cf. also Delalande S. A., DE 2836305, 1979; Chem. Abstr. 90, 186926):

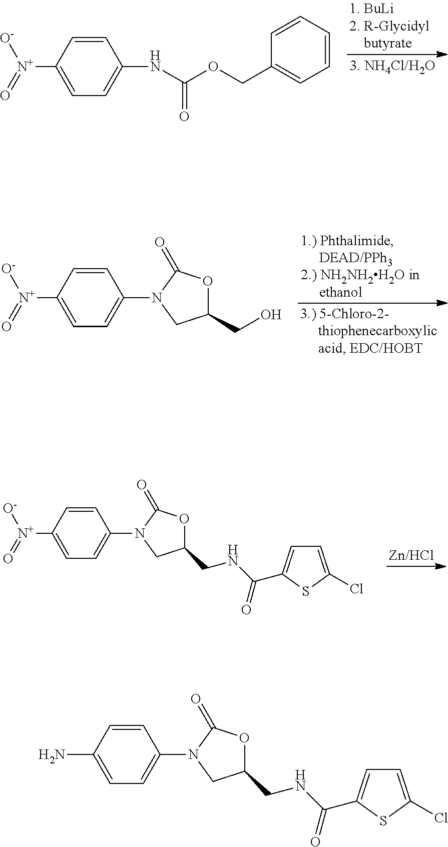 [SMILES: O=C(Nc1ccc([N+](=O)[O-])cc1)OCc1ccccc1 ; O=C1O[C@@H](CO)CN1c1ccc([N+](=O)[O-])cc1 ; O=C(NC[C@H]1CN(c2ccc([N+](=O)[O-])cc2)C(=O)O1)c1ccc(Cl)s1 ; Nc1ccc(N2C[C@H](CNC(=O)c3ccc(Cl)s3)OC2=O)cc1]

### Example 150

### 5-Chloro-N-({3-[4-(glycylamino)phenyl]-2-oxo-1,3-oxazolidin-5-yl}methyl)-2-thiophenecarboxamide

starting from Example 152:

MS (ES-pos): m/z (%)=408 (100);

HPLC (method 3): rt (%)=3.56 (97).

IC₅₀: 2 μM

### Example 151

### 5-(Aminomethyl)-3-[4-(2-oxo-1-pyrrolidinyl)phenyl]-1,3-oxazolidin-2-one

starting from Example 60:

MS (ESI): m/z (%)=276 (M+H, 100);

HPLC (method 3): rt (%)=2.99 (100).

IC₅₀:2 μM

Examples 152 to 166 which follow relate to the amino group-derivatization of aniline- or benzylamine-substituted oxazolidinones with various reagents:

### Example 152

### 5-(Chloro-N-({3-[4-(N-tert-butyloxycarbonylglycylamino)phenyl]-2-oxo-1,3-oxazolidin-5-yl}methyl)-2-thiophenecarboxamide

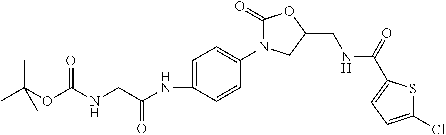 [SMILES: CC(C)(C)OC(=O)NCC(=O)Nc1ccc(N2CC(CNC(=O)c3ccc(Cl)s3)OC2=O)cc1]

754 mg (2.1 mmol) of N-{[3-(4-aminophenyl)-2-oxo 1,3-oxazolidin-5-yl]methyl}-5-chloro-2-thiophenecarboxamide (from Example 149) are added to a solution of 751 mg (4.3 mmol) of Boc-glycine, 870 mug (6.4 mmol) of HOBT (1-hydroxy-1H-benzotriazole×H₂O), 1790 mg (4.7 mmol) of HBTU [O-(benzotriazol-1-yl)-N,N,N′,N′-tetramethyluronium hexafluorophosphate] and 1.41 ml (12.9 mmol) of N-methylmorpholine in 15 ml of DMF/CH₂Cl₂ (1:1) at 0° C. The mixture is stirred at room temperature overnight before being diluted with water. The precipitated solid is filtered off and dried. Yield: 894 mg (79.7% of theory);

MS (DCI, NH₃): m/z (%)=526 (M+NH₄, 100);

HPLC (method 3): rt (%)=4.17 (97).

### Example 153

### N-[(3-{4-[(Acetylamino)methyl]phenyl}-2-oxo-1,3-oxazolidin-5-yl)methyl]-5-chloro-2-thiophenecarboxamide

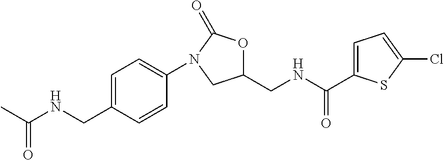 [SMILES: CC(=O)NCc1ccc(N2CC(CNC(=O)c3ccc(Cl)s3)OC2=O)cc1]

Acetic anhydride (0.015 ml, 0.164 mmol) is added to a mixture of 30 mg (0.082 nmol) of N-({3-[4-(aminomethyl)phenyl]-2-oxo-1,3-oxazolidin-5-yl}methyl)-5-chloro-2-thiophenecarboxamide (from Example 148) in 1.5 ml of absolute THF and 1.0 ml of absolute dichloromethane, 0.02 ml of absolute pyridine at 0° C. The mixture is stirred at room temperature overnight. The product is obtained after addition of ether and crystallization. Yield: 30 mg (87% of theory).

MS (ESI): m/z (%)=408 (M+H, 18), 305 (85);

HPLC (method 1): rt (%)=3.78 (97).

IC₅₀: 0.6 μM

### Example 154

### N-{[3-(4-{[(Aminocarbonyl)amino]methyl}phenyl)-2-oxo-1,3-oxazolidin-5-yl]methyl}-5-chloro-2-thiophenecarboxamide

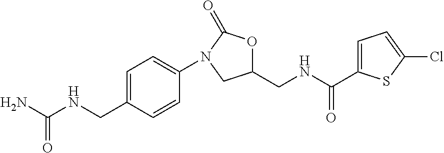 [SMILES: NC(=O)NCc1ccc(N2CC(CNC(=O)c3ccc(Cl)s3)OC2=O)cc1]

0.19 ml (0.82 mmol) of trimethylsilyl isocyanate is added dropwise to a mixture of 30 mg (0.082 mmol) of N-((3-[4-(aminomethyl)phenyl]-2-oxo-1,3-oxazolidin-5-yl)methyl)-5-chloro-2-thiophenecarboxamide (from Example 148) in 1.0 ml of dichloromethane at room temperature. The mixture is stirred overnight before, after addition of ether, the product is obtained by filtration.

Yield: 21.1 mg (52% of theory),

MS (ESI): m/z (%)=409 (M+H, 5), 305 (72);

HPLC (method 1): rt (%)=3.67 (83).

IC₅₀: 1.3 μM

### General Method for acylating N-{[3-(4-aminophenyl)-2-oxo-1,3-oxazolidin-5-yl]methyl}-5-chloro-2-thiophenecarboxamide with Carbonyl Chlorides

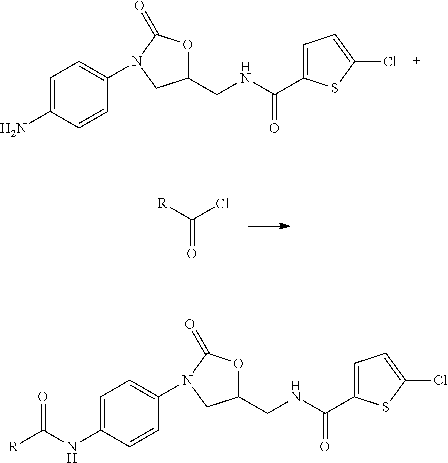

An approx. 0.1 molar solution of N-{[3-(4-aminophenyl)-2-oxo-1,3-oxazolidin-5-yl]methyl}-5-chloro-2-thiophenecarboxamide (from Example 149) (1.0 eq.) in absolute dichloromethane/pyridine (19:1) is added dropwise under argon to the appropriate acid chloride (2.5 eq.). The mixture is stirred overnight before addition of approx. 5 eq of PS-trisamine (Argonaut Technologies) and 2 ml of absolute dichloromethane. Gentle stirring for 1 h is followed by filtration and concentration of the filtrate. The products are purified where appropriate by preparative RP-HPLC.

The following were prepared in an analogous manner:

### Example 155

### N-({3-[4-(Acetylamino)phenyl]-2-oxo-1,3-oxazolidin-5-yl}methyl)-5-chloro-2-thiophenecarboxamide

LC-MS: m/z 4%)=394 (M+H, 100);

LC-MS (method 6): rt (%)=3.25 (100).

IC₅₀: 1.2 μM

### Example 156

### 5-Chloro-N-[(2-oxo-3-{4-[(2-thienylcarbonyl)amino]phenyl}-1,3-oxazolidin-5-yl)methyl]-2-thiophenecarboxamide

LC-MS: m/z (%)=462 (M+H, 100);

LC-MS (method 6): rt (%)=3.87 (100).

IC₅₀: 1.3 μM

### Example 157

### 5-Chloro-N-[(3-{4-[(methoxyacetyl)amino]phenyl}-2-oxo-1,3-oxazolidin-5-yl)methyl]2-thiophenecarboxamide

LC-MS: m/z (%)=424 (M+H, 100);

LC-MS (method 6): rt (%)=3.39 (100).

IC₅₀: 0.73 μM

### Example 158

### N-{4-[5-({[(5-Chloro-2-thienyl)carbonyl]amino}methyl)-2-oxo-1,3-oxazolidin-3-yl]phenyl}-3,5-dimethyl-4-isoxazolecarboxamide

LC-MS: m/z (%)=475 (M+H, 100).

IC₅₀: 0.46 μM

### Example 159

### 5-Chloro-N-{[3-(4-{[(3-chloropropyl)sulfonyl]amino}phenyl)-2-oxo-1,3-oxazolidin-5-yl]methyl}-2-thiophenecarboxamide

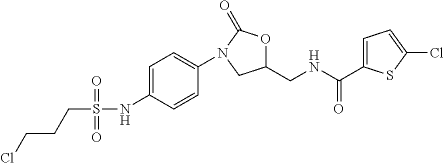 [SMILES: O=C(NCC1CN(c2ccc(NS(=O)(=O)CCCCl)cc2)C(=O)O1)c1ccc(Cl)s1]

35 mg (0.1 mmol) of N-{[3-(4-aminophenyl)-2-oxo-1,3-oxazolidin-5-yl]methyl}-5-chloro-2-thiophenecarboxamide (from Example 149) are added to an ice-cooled solution of 26.4 mg (0.15 mmol) of 3-chloro-1-propanesulfonyl chloride and 0.03 ml (0.2 mmol) of triethylamine in 3.5 ml of absolute dichloromethane. After 30 min, the ice cooling is removed and the mixture is stirred at room temperature overnight before adding 150 mg (approx. 5.5 eq) of PS-trisamine (Argonaut Technologies) and 0.5 ml of dichloromethane. The suspension is stirred gently for 2 h and filtered (the resin is washed with dichloromethane/methanol), and the filtrate is concentrated. The product is purified by preparative RP-HPLC. Yield: 19.6 mg (40% of theory),

LC-MS: m/z (%)=492 (M+H, 100);

LC-MS (method 5): rt (%)=3.82 (91).

IC₅₀: 1.7 μM

### Example 160

### 5-Chloro-N-({3-[4-(1,1-dioxido-2-isothiazolidinyl)phenyl]-2-oxo-1,3-oxazolidin-5-yl}methyl)-2-thiophenecarboxamide

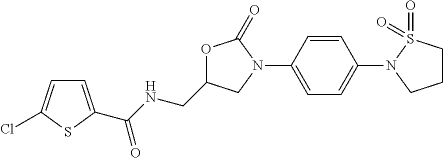 [SMILES: O=C(NCC1CN(c2ccc(N3CCCS3(=O)=O)cc2)C(=O)O1)c1ccc(Cl)s1]

A mixture of 13.5 mg (0.027 mmol) of 5-chloro-N-{[3-(4-{[(3-chloropropyl)sulfonyl]amino}phenyl)-2-oxo-1,3-oxazolidin-5-yl]methyl}-2-thiophenecarboxamide (from Example 159) and 7.6 mg (0.055 mmol) of potassium carbonate in 0.2 ml of DMF is heated at 100° C. for 2 h. Cooling is followed by dilution with dichloromethane and washing with water. The organic phase is dried and concentrated. The residue is purified by preparative thin-layer chromatography (silica gel, dichloromethane/methanol, 95:5). Yield: 1.8 mg (14.4% of theory).

MS (ESI): mm/z (%)=456 (M+H, 15), 412 (100);

LC-MS (method 4): rt (%)=3.81 (90).

IC₅₀: 0.14 M

### Example 161

### 5-Chloro-N-[((5S)-3-{4-[(5-chloropentanoyl)amino]phenyl}-2-oxo-1,3-oxazolidin-5-yl)methyl]-2-thiophenecarboxamide

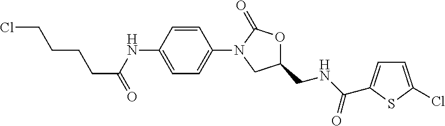 [SMILES: O=C(CCCCCl)Nc1ccc(N2C[C@H](CNC(=O)c3ccc(Cl)s3)OC2=O)cc1]

0.5 g (1.29 mmol) of N-{[(5S)-3-(4-aminophenyl)-2-oxo-1,3-oxazolidin-5-yl]methyl}-5-chloro-2-thiophenecarboxamide (from Example 149) is dissolved in 27 ml of tetrahydrofuran, and 0.2 g (1.29 mmol) of 5-chlorovaleryl chloride and 0.395 ml (2.83 mmol) of triethylamine are added. The mixture is evaporated in vacuo and chromatographed on silica gel with a toluene/ethyl acetate=1:1->ethyl acetate gradient. 315 mg (52% of theory) of a solid are obtained.

M.p.: 211° C.

### Example 162

### 5-Chloro-N-({(5S)-2-oxo-3-[4-(2-oxo-1-piperidinyl)phenyl]-1,3-oxazolidin-5-yl}methyl)-2-thiophenecarboxamide

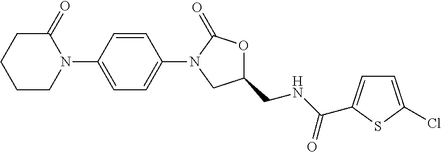 [SMILES: O=C(NC[C@H]1CN(c2ccc(N3CCCCC3=O)cc2)C(=O)O1)c1ccc(Cl)s1]

30 mg of 60 percent NaH in liquid paraffin are added under inert conditions to 5 ml of DMSO, and the mixture is heated at 75° C. for 30 min until gas evolution ceases. Then a solution of 290 mg (0.617 mmol) of 5-chloro-N-[((5S)-3-{4-[(5-chloropentanoyl)amino]phenyl}-2-oxo-1,3-oxazolidin-5-yl)methyl]-2-thiophenecarboxamide (from Example 161) in 5 ml of methylene chloride is added dropwise, and the mixture is stirred at room temperature overnight. The reaction is stopped and the mixture is added to 100 ml of water and extracted with ethyl acetate. The evaporated organic phase is chromatographed on an RP-8 column and eluted with acetonitrile/water. 20 mg (7.5% of theory) of the target compound are obtained.

M.p.: 205° C.;

NMR (300 MHz, d₆-DMSO): δ=1.85 (m, 4H), 2.35 (m, 2H), 3.58 (m, 4H), 3.85 (m, 1H), 4.2 (t, 1H), 4.82 (m, 1H), 7.18 (d, 1H, thiophene), 7.26 (d, 2H), 7.5 (d, 2H), 2.68 (d, 1H, thiophene), 9.0 (t, 1H, CONH).

IC₅₀: 2.8 nM

### Example 163

### 5-Chloro-N-[((S)-3-{4-[(3-bromopropionyl)amino]phenyl}-2-oxo-1,3-oxazolidin-5-yl)methyl]-2-thiophenecarboxamide

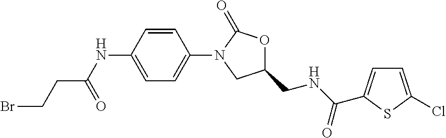 [SMILES: O=C(CCBr)Nc1ccc(N2C[C@H](CNC(=O)c3ccc(Cl)s3)OC2=O)cc1]

is obtained in an analogous manner from Example 149.

### Example 164

### 5-Chloro-N-({(5S)-2-oxo-3-[4-(2-oxo-1-azetidinyl)phenyl]-1,3-oxazolidin-5-yl}methyl)-2-thiophenecarboxamide

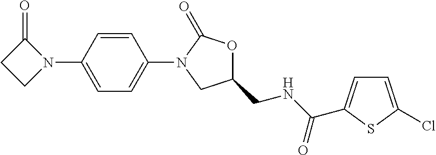 [SMILES: O=C(NC[C@H]1CN(c2ccc(N3CCC3=O)cc2)C(=O)O1)c1ccc(Cl)s1]

is obtained in an analogous manner by cyclization of the open-chain bromopropionyl compound from Example 163 using NaH/DMSO.

MS (ESI): m/z (%)=406 ([M+H]⁺, 100), Cl pattern.

IC₅₀: 380 nM

### Example 165

### tert-Butyl 4-{4-[5-({[(5-chloro-2-thienyl)carbonyl]amino}methyl)-2-oxo-1,3-oxazolidin-3-yl]phenyl}-3,5-dioxo-1-piperazinecarboxylate

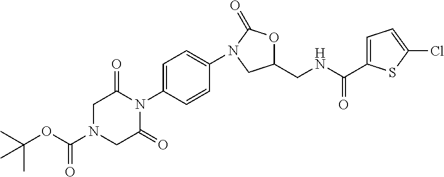 [SMILES: CC(C)(C)OC(=O)N1CC(=O)N(c2ccc(N3CC(CNC(=O)c4ccc(Cl)s4)OC3=O)cc2)C(=O)C1]

300 mg (0.85 nmol) of N-([3-(4-aminophenyl)-2-oxo-1,3-oxazolidin-5-yl]-methyl)-5-chloro-2-thiophenecarboxamide in 6 ml of a mixture of DMF and dichloromethane (1:1) are added to a solution of 199 mg (0.85 mmol) of Boc-iminodiacetic acid, 300 mg (2.2 mmol) of HOBT, 0.66 ml (6 mmol) of N-methylmorpholine and 647 mg (1.7 mmol) of HBTU. The mixture is stirred overnight before, after dilution with dichloromethane, being washed with water, saturated ammonium chloride solution, saturated sodium bicarbonate solution, water and saturated sodium chloride solution. The organic phase is dried over magnesium sulfate and concentrated. The crude product is purified by chromatography on silica gel (dichloromethane/methanol 98:2). Yield: 134 mg (29% of theory):

MS (ESI): m/z (%)=571 (M+Na, 82), 493 (100);

HPLC (method 3): rt (%)=4.39 (90).

IC₅₀: 2 μM

### Example 166

### N-[((5S)-3-{4-[(3R)-3-Amino-2-oxo-1-pyrrolidinyl]phenyl}-2-oxo-1,3-oxazolidin-5-yl)methyl]-5-chloro-2-thiophenecarboxamide trifluoroacetate

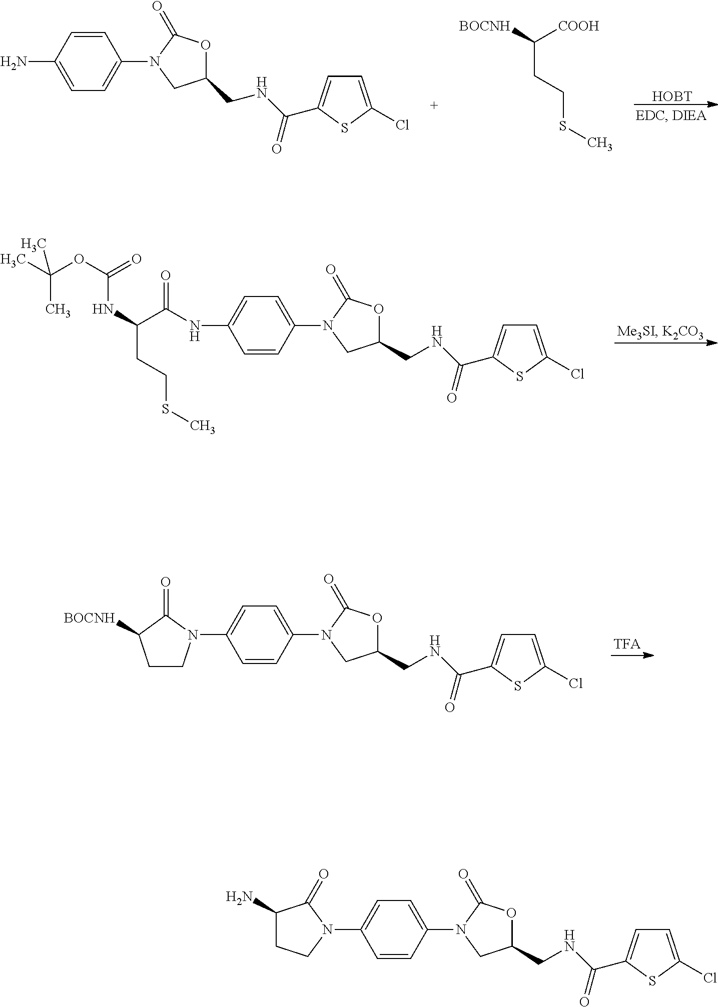

### N2-(tert-Butoxycarbonyl)-N1-{4-[(5S)-5-({[(5-chloro-2-thienyl)carbonyl]amino}methyl)-2-oxo-1,3-oxazolidin-3-yl]phenyl}-D-methioninamide

429 mg (1.72 mmol) of N-BOC-D-methionine, 605 mg (1.72 mmol) of N-{[(5S)-3-(4-aminophenyl)-2-oxo-1,3-oxazolidin-5-yl]methyl}-5-chloro-2-thiophenecarboxamide and 527 mg (3.44 mmol) of HOBT hydrate are dissolved in 35 ml of DMF, and 660 mg (3.441 mmol) of EDCI hydrochloride and then, dropwise, 689 mg (5.334 mmol) of N-ethyldiisopropylamine are added. The mixture is stirred at room temperature for two days. The resulting suspension is filtered with suction and the residue is washed with DMF. The combined filtrates are mixed with a little silica gel, evaporated in vacuo and chromatographed on silica gel with a toluene->T10EA7 gradient. 170 mg (17% of theory) of the target compound are obtained with a melting point of 183° C.

R_{f} (SiO₂, toluene/ethyl acetate=1:1):0.2.

¹H-NMR (300 MHz, d₆-DMSO): δ=1.4 (s, 1H, BOC), 1.88-1.95 (m, 2H), 2.08 (s, 3H, SMe), 2.4-2.5 (m, 2H, partly covered by DMSO), 3.6 (m, 2H), 3.8 (m, 1H), 4.15 (m, 2H), 4.8 (m, 1H), 7.2 (1H, thiophene), 7.42 (d, part of an AB system, 2H), 7.6 (d, part of an AB system, 2H), 7.7 (d, 1H, thiophene), 8.95 (t, 1H, CH₂NHCO), 9.93 (bs, 1H, NH).

### tert-Butyl (3R)-1-{4-[(5S)-5-({[(5-chloro-2-thienyl)carbonyl]amino}methyl)-2-oxo-1,3-oxazolidin-3-yl]phenyl}-2-oxo-3-pyrrolidinylcarbamate

170 mg (0.292 mmol) of N2-(tert-butoxycarbonyl)-N1-{4-[(5S)-5-({[(5-chloro-2-thienyl)carbonyl]amino}methyl)-2-oxo-1,3-oxazolidin-3-yl]phenyl}-D-methioninamide are dissolved in 2 ml of DMSO, and 178.5 mg (0.875 mmol) of trimethylsulfonium iodide and 60.4 mg (0.437 mmol) of potassium carbonate are added, and the mixture is stirred at 80° C. for 3.5 hours. It is then evaporated under high vacuum, and the residue is washed with ethanol. 99 mg of the target compound remain.

¹H-NMR (300 MHz, d₆-DMSO): δ=1.4 (s, 1H, BOC), 1.88-2.05 (m, 1H), 2.3-2.4 (m, 1H), 3.7-3.8 (m, 3H), 3.8-3.9 (m, 1H), 4.1-4.25 (m, 1H), 4.25-4.45 (m, 1H), 4.75-4.95 (m, 1H), 7.15 (1H, thiophene), 7.25 (d, 1H), 7.52 (d, part of an AB system, 2H), 7.65 (d, part of an AB system, 2H), 7.65 (d, 1H, thiophene), 9.0 (broad s, 1H).

### N-[((5S)-3-{4-[(3R)-3-Amino-2-oxo-1-pyrrolidinyl]phenyl}-2-oxo-1,3-oxazolidin-5-yl)methyl]-5-chloro-2-thiophenecarboxamide trifluoroacetate

97 mg (0.181 mmol) of tert-butyl (3R)-1-{4-[(5S)-5-({[(5-chloro-2-thienyl)carbonyl]amino}methyl)-2-oxo-1,3-oxazolidin-3-yl]phenyl}-2-oxo-3-pyrrolidinylcarbamate are suspended in 4 ml of methylene chloride and, after addition of 1.5 ml of trifluoroacetic acid, stirred at room temperature for 1 hour. The mixture is then evaporated in vacuo and purified on an RP-HPLC (acetonitril/water/0.1% TFA gradient). Evaporation of the relevant fraction results in 29 mg (37% of theory) of the target compound with a melting point of 241° C. (decomposition).

R_{f} (SiO₂EtOH/TEA=17:1) 0.19.

¹H-NMR (300 MHz, d₆-DMSO): δ=1.92-2.2 (m, 1H), 2.4-2.55 (m, 1H, partially covered by DMSO peak), 3.55-3.65 (m, 2H), 3.75-3.95 (m, 3H), 4.1-4.3 (m, 2H), 4.75-4.9 (m, 1H), 7.2 (1H, thiophene), 7.58 (d, part of an AB system, 2H), 7.7 (d, part of an AB system, 2H), 7.68 (d, 1H, thiophene), 8.4 (broad s, 3H, NH3), 8.9 (t, 1H, NHCO).

Examples 167 to 170 which follow relate to the introduction of sulfonamide groups into phenyl-substituted oxazolidinones:

### General Method for Preparing Substituted Sulfonamides Starting from 5-chloro-N-[(2-oxo-3-phenyl-1,3-oxazolidin-5-yl)methyl]-2-thiophenecarboxamide

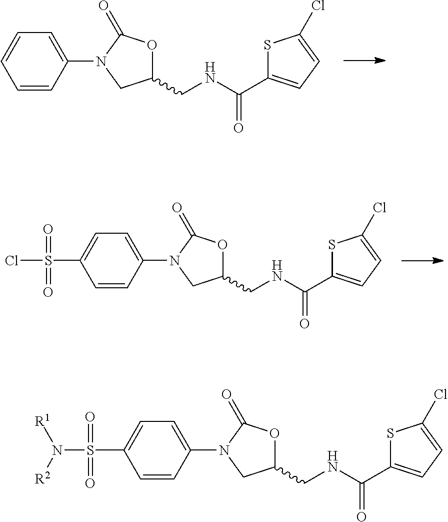

5-Chloro-N-[(2-oxo-3-phenyl-1,3-oxazolidin-5-yl)methyl]-2-thiophenecarboxamide (from Example 96) is added to chlorosulfonic acid (12 eq.) under argon at 5° C. The reaction mixture is stirred at room temperature for 2 h and then added to ice-water. The precipitate which separates out is filtered, washed with water and dried.

It is then dissolved in tetrahydrofuran (0.1 mol/l) under argon at room temperature, and the appropriate amine (3 eq.), triethylamine (1.1 eq.) and dimethylaminopyridine (0.1 eq.) are added. The reaction mixture is stirred for 1-2 h and then concentrated in vacuo. The desired product is purified by flash chromatography (dichloromethane/methanol mixtures).

The following were prepared in an analogous manner:

### Example 167

### 5-Chloro-N-({2-oxo-3-[4-(1-pyrrolidinylsulfonyl)phenyl]-1,3-oxazolidin-5-yl}methyl)-2-thiophenecarboxamide

MS (ESI): m/z (%)=492 ([M+Na]⁺, 100), 470 ([M+H]⁺, 68), Cl pattern;

HPLC (method 3): rt (%)=4.34 (100).

IC₅₀: 0.5 μM

### Example 168

### 5-Chloro-N-[3-{4-[(4-methyl-1-piperazinyl)sulfonyl]phenyl}-2-oxo-1,3-oxazolidin-5-yl)methyl]-2-thiophenecarboxamide

MS (ESI): m/z (%)=499 ([M+H]⁺, 100), Cl pattern;

HPLC (method 2): rt (%)=3.3 (100).

### Example 169

### 5-Chloro-N-({2-ox-3-[4-(1-piperidinylsulfonyl)phenyl]-1,3-oxazolidin-5-yl}methyl)-2-thiophenecarboxamide

MS (ESI): m/z (%)=484 ([M+H]⁺, 100). Cl pattern;

HPLC (method 2): rt (%)=4.4 (100).

### Example 170

### 5-Chloro-N-[(3-{4-[(4-hydroxy-1-piperidinyl)sulfonyl]phenyl}-2-oxo-1,3-oxazolidin-5-yl)methyl]-2-thiophenecarboxamide

MS (ESI): m/z (%)=500) ([M+H]⁺, 100), Cl pattern;

HPLC (method 3): rt 1%)=3.9 (100).

### Example 71

### 5-Chloro-N-{2-oxo-3-[4-(1-pyrrolidinyl)phenyl]-1,3-oxazolidin-5-yl}methyl)-2-thiophenecarboxamide

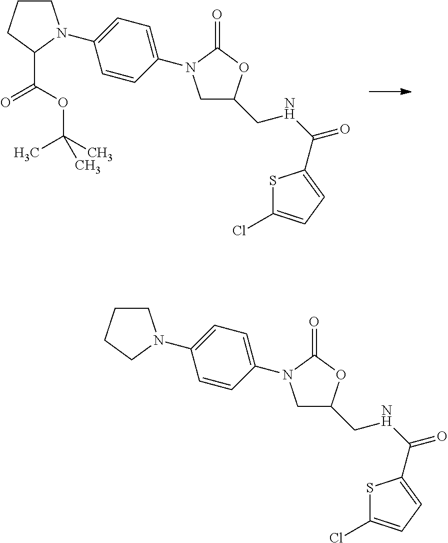 [SMILES: CC(C)(C)OC(=O)C1CCCN1c1ccc(N2CC(CNC(=O)c3ccc(Cl)s3)OC2=O)cc1 ; O=C(NCC1CN(c2ccc(N3CCCC3)cc2)C(=O)O1)c1ccc(Cl)s1]

780 mg (1.54 mmol) of tert-butyl 1-{4-[5-({[(5-chloro-2-thienyl)carbonyl]amino}methyl)-2-oxo-1,3-oxazolidin-3-yl]phenyl}prolinate are dissolved in 6 ml of dichloromethane and 9 ml of trifluoroacetic acid, and the mixture is stirred at 40° C. for two days. The reaction mixture is then concentrated and stirred with ether and 2 N sodium hydroxide solution. The aqueous phase is concentrated and stirred with ether and 2 N hydrochloric acid. The organic phase from this extraction is dried over MgSO₄, filtered and concentrated. The crude product is chromatographed on silica gel (CH₂Cl/EtOH/conc. aq. NH₃ solution=100/1/0.1 to 20/1/0.1).

280 mg (40% of theory) of the product are obtained.

MS (ESI): m/z (%)=406 (M+H, 100);

HPLC (method 4): rt=3.81 min.

HPLC Parameters and LC-MS Parameters for the HPLC and LC-MS Data Stated in the Preceding Examples (the Unit of Retention Time (Rt) is Minutes):

[1] Column: Kromasil C18, L-R temperature: 30° C., flow rate=0.75 mlmin⁻¹, eluent: A=0.01 M HClO₄, B═CH₃CN, gradient: ->0.5 min 98% A->4.5 min 10% A->6.5 min 10% A

[2] Column: Kromasil C18 60*2, L-R temperature: 30° C., flow rate=0.75 mlmin⁻¹, eluent: A=0.01 M H₃PO₄, B═CH₃CN, gradient: ->0.5 min 90% A->4.5 min 10% A->6.5 min 10% A

[3] Column: Kromasil C18 60*2, L-R temperature: 30° C., flow rate=0.75 mlmin⁻¹, eluent: A=0.005 M HClO₄, B═CH₃CN, gradient: ->0.5 min 98% A->4.5 min 10% A->6.5 min 10% A

[4] Column: Symmetry C18 2.1×150 mm, column oven: 50° C., flow rate=0.6 mlmin⁻¹, eluent: A=0.6 g of 30% HCl/l of water. B═CH₃CN, gradient: 0.0 min 90% A->4.0 min 10% A->9 min 10% A

[5] MHZ-2Q, Instrument Micromass Quattro LCZ

Column Symmetry C18, 50 mm×2.1 mm, 3.5 m, temperature: 40° C., flow rate=0.5 ml min⁻¹, eluent A=CH₃CN+0.1% formic acid, eluent B=water+0.1% formic acid, gradient: 0.0 min 10% A->4 min 90% A->6 min 90% A

[6] MHZ-2P. Instrument Micromass Platform LCZ

Column Symmetry C18, 50 mm×2.1 mm, 3.5 μm, temperature: 40° C., flow rate=0.5 mlmin⁻¹, eluent A=CH₃CN+0.1% formic acid, eluent B=water+0.1% formic acid, gradient: 0.0 min 10% A->4 min 90% A->6 min 90% A

[7] MHZ-7Q, Instrument Micromass Quattro LCZ

Column Symmetry C18, 50 mm×2.1 mm, 3.5 μm, temperature: 40° C., flow rate=0.5 mlmin⁻¹, eluent A=CH₃CN+0.1% formic acid, eluent B=water+0.1% formic acid, gradient: 0.0 min 5% A->1 min 5% A->5 min 90% A->6 min 90% A

General Method for Preparing Oxazolidinones of the General Formula B by Solid Phase-Assisted Synthesis

Reactions with various resin-bound products look place in a set of separate reaction vessels.

5-(Bromomethyl)-3-(4-fluoro-3-nitrophenyl)-1,3-oxazolidin-2-one A (prepared from epibromohydrin and 4-fluoro-3-nitrophenyl isocyanate with LiBr/Bu₃PO in xylene in analogy to U.S. Pat. No. 4,128,654, Ex.2) (1.20 g 3.75 mmol) and ethyldiisoproylamine (DIEA, 1.91 ml, 4.13 mmol) were dissolved in DMSO (70 ml), mixed with a secondary amine (1.1 eq, amine component 1) and reacted at 55° C. for 5 h. TentaGel SAM resin (5.00 g, 0.25 mmol/g) was added to this solution and reacted at 75° C. for 48 h. The resin was filtered and repeatedly washed with methanol (MeOH), dimethylformamide (DMF). MeOH, dichloromethane (DCM) and diethyl ether and dried. The resin (5.00 g) was suspended in dichloromethane (80 ml), mixed with DIEA (10 eq) and 5-chlorothiophene-2-carbonyl chloride [prepared by reacting 5-chlorothiophene-2-carboxylic acid (5 eq) and 1-chloro-1-dimethylamino-2-methylpropene (5 eq) in DCM (20 ml) at room temperature for 15 minutes] and reacted at room temperature for 5 h. The resulting resin was filtered and washed repeatedly with MeOH, DCM and diethyl ether and dried. The resin was then suspended in DMF/water (v/v 9:2, 80 ml), mixed with SnCl₂*2H₂O (5 eq) and reacted at room temperature for 18 h. The resin was again washed repeatedly with MeOH, DMF, water, MeOH, DCM and diethyl ether and dried. This resin was suspended in DCM, mixed with DIEA (10 eq) and, at 0° C., with an acid chloride (5 eq of acid derivative 1) and reacted at room temperature overnight. Before the reaction, carboxylic acids were converted into the corresponding acid chlorides by reacting with 1-dimethylamino-1-chloro-2-methylpropene (1 eq, based on the carboxylic acid) in DCM at room temperature for 15 min. The resin was washed repeatedly with DMF, water, DMF, MeOH, DCM and diethyl ether and dried. Where Fmoc-protected amino acids were used as acid derivative 1, the Fmoc-protective group was eliminated in the last reaction step by reacting with piperidine/DMF (v/v, 1/4) at room temperature for 15 minutes, and the resin was washed with DMF, MeOH, DCM and diethyl ether and dried. The products were then cleaved off the solid phase with trifluoroacetic acid (TFA)/DCM (v/v, 1/1), the resin was filtered off, and the reaction solutions were evaporated. The crude products were filtered through silica gel (DCM/MeOH, 9:1) and evaporated in order to obtain a set of products B.

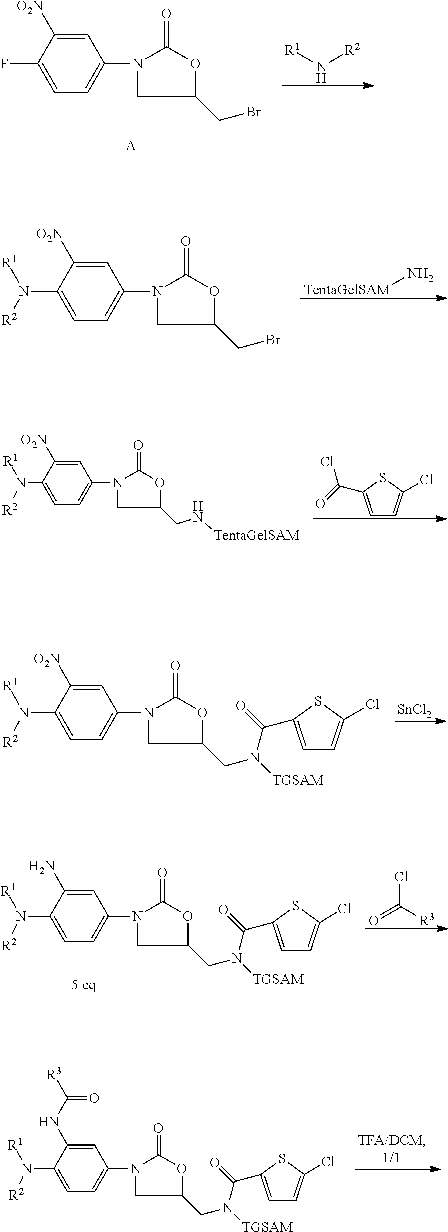

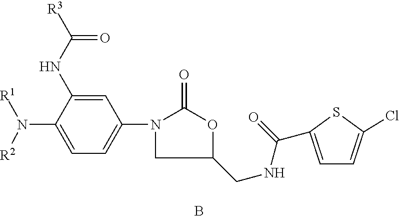

### Compounds Prepared by Solid Phase-Assisted Synthesis

### Example 172

### N-({3-[3-Amino-4-(1-pyrrolidinyl)phenyl]-2-oxo-1,3-oxazolidin-5-yl}methyl)-5-chloro-2-thiophenecarboxamide

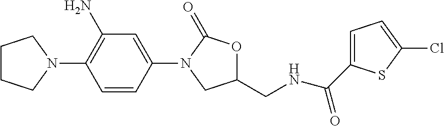 [SMILES: Nc1cc(N2CC(CNC(=O)c3ccc(Cl)s3)OC2=O)ccc1N1CCCC1]

5 g (1.25 mmol) of TentaGel SAM resin were reacted with pyrrolidine as amine derivative 1 in analogy to the general procedure for preparing the derivatives B. The aniline obtained after reduction with SnCl₂*2H₂ was eliminated from the solid phase, without a further acylation step, and evaporated. The crude product was partitioned between ethyl acetate and NaHCO₃ solution, and the organic phase was salted out with NaCl, decanted and evaporated to dryness. This crude product was purified by vacuum flash chromatography on silica gel (dichloromethane/ethyl acetate, 3:1-1:2).

¹H-NMR (300 MHz, CDCl₃): 1.95-2.08, br, 4H; 3.15-3.30, br, 4H; 3.65-3.81, m, 2H; 3.89, ddd, 1H; 4.05, dd, 1H; 4.81, dddd, 1H; 6.46, dd, 1H; 6.72, dd, 1H; 6.90, dd, 1H; 6.99, dd, 1H; 7.03, dd, 1H; 7.29, d, 1H.

### Example 173

### N-[(3-{3-(β-Alanylamino)-4-[(3-hydroxypropyl)amino]phenyl}-2-oxo-1,3-oxazolidin-5-yl)methyl]-5-chloro-2-thiophenecarboxamide

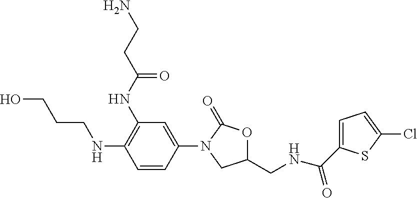 [SMILES: NCCC(=O)Nc1cc(N2CC(CNC(=O)c3ccc(Cl)s3)OC2=O)ccc1NCCCO]

5 g (1.25 mmol) of TentaGel SAM resin were reacted with azetidine as amine derivative 1 and Fmoc-β-alanine as acid derivative 1 in analogy to the general procedure for preparing the derivatives B. The crude product obtained after elimination was stirred in methanol at room temperature for 48 h and evaporated to dryness. This crude product was purified by reversed phase HPLC with a water/TFA/acetonitrile gradient.

¹H-NMR (400 MHz, CD₃OD): 2.31, tt, 2H; 3.36, t, 2H; 3.54, t, 2H; 3.62, t, 2H; 3.72, dd, 1H; 3.79, dd, 1H; 4.01, dd, 1H; 4.29, dd, 2H; 4.43, t, 2H; 4.85-4.95, m, 1H; 7.01, d, 1H; 4.48-7.55, m, 2H; 7.61, d, 1H; 7.84, d, 1H.

### Example 174

### N-({3-[4-(3-Amino-1-pyrrolidinyl)-3-nitrophenyl]-2-oxo-1,3-oxazolidin-5-yl}methyl)-5-chloro-2-thiophenecarboxamide

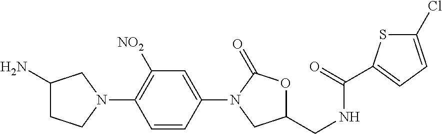 [SMILES: NC1CCN(c2ccc(N3CC(CNC(=O)c4ccc(Cl)s4)OC3=O)cc2[N+](=O)[O-])C1]

130 mg (32.5 μmol) of TentaGel SAM resin were reacted with tert-butyl 3-pyrrolidinylcarbamate as amine derivative 1 in analogy to the general procedure for preparing the derivatives B. The nitrobenzene derivative obtained after acylation with 5-chlorothiophenecarboxylic acid was eliminated from the solid phase and evaporated. This crude product was purified by reversed phase HPLC with a water/TFA/acetonitrile gradient.

¹H-NMR (400 MHz, CD₃OH): 2.07-2.17, m, 1H; 2.39-2.49, m, 1H; 3.21-3.40, m, 2H; 3.45, dd, 1H; 3.50-3.60, m, 1H; 3.67, dd, 1H; 3.76, dd, 1H; 3.88-4.00, m, 2H; 4.14-4.21, t, 1H; 4.85-4.95, m, 1H; 7.01, d, 1H; 7.11, d, 1H; 7.52, d, 1H; 7.66, dd, 1H; 7.93, d, 1H.

### Example 175

### N-({3-[3-amino-4-(1-piperidinyl)phenyl]-2-oxo-1,3-oxazolidin-5-yl}methyl)-5-chloro-2-thiophenecarboxamide

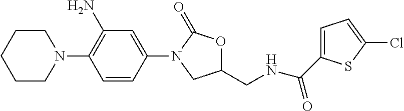 [SMILES: Nc1cc(N2CC(CNC(=O)c3ccc(Cl)s3)OC2=O)ccc1N1CCCCC1]

130 mg (32.5 μmol) of TentaGel SAM resin were reacted with piperidine as amine derivative 1 in analogy to the general procedure for preparing the derivatives B. The aniline obtained after reduction was eliminated, without a further acylation step, from the solid phase and evaporated. This crude product was purified by reversed phase HPLC with a water/TFA/acetonitrile gradient.

¹H-NMR (400 MHz, CD₃OH): 1.65-1.75, m, 2H; 1.84-1.95, m, 4H; 3.20-3.28, m, 4H; 3.68, dd, 1H; 3.73, dd, 1H; 3.90, dd, 1H; 4.17, dd, 1H; 4.80-4.90, m, 1H; 7.00, d, 1H; 7.05, dd, 1H; 7.30-7.38, m, 2H; 7.50, d, 1H.

### Example 176

### N-({3-[3-(Acetylamino)-4-(1-pyrrolidinyl)phenyl]-2-oxo-1,3-oxazolidin-5-yl}methyl)-5-chloro-2-thiophenecarboxamide

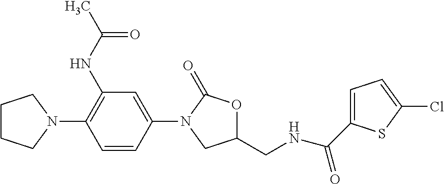 [SMILES: CC(=O)Nc1cc(N2CC(CNC(=O)c3ccc(Cl)s3)OC2=O)ccc1N1CCCC1]

130 mg (32.5 μmol) of TentaGel SAM resin were reacted with pyrrolidine as amine derivative 1 and acetyl chloride as acid derivative 1 in analogy to the general procedure for preparing the derivatives B. The crude product was partitioned between ethyl acetate and NaHCO₃ solution, and the organic phase was salted out with NaCl, decanted and evaporated to dryness. This crude product was purified by vacuum flash chromatography on silica gel (dichloromethane/ethyl acetate, 1:1-0.1).

¹H-NMR (400 MHz, CD₃OH): 1.93-2.03, br, 4H; 2.16, s, 3H; 3.20-3.30, br, 4H; 3.70, d, 2H; 3.86, dd, 1H; 4.10, dd, 1H; 4.14, dd, 1H; 4.80-4.90, m, 1H; 7.00, d, 1H; 7.07, d, 1H; 7.31, dd, 1H; 7.51, d, 1H; 7.60, d, 1H.

The following compounds were prepared in analogy to the general procedure.

| | | | HPLC |
|---|---|---|---|
| Example | Structure | Ret. time | [%] |
| 177 | 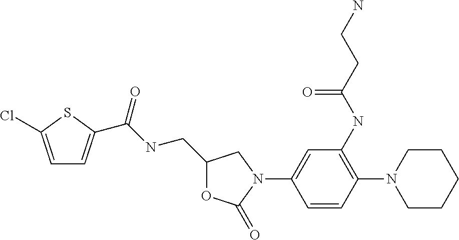 [SMILES: NCCC(=O)Nc1cc(N2CC(CNC(=O)c3ccc(Cl)s3)OC2=O)ccc1N1CCCCC1] | 2.62 | 79.7 |
| 178 | 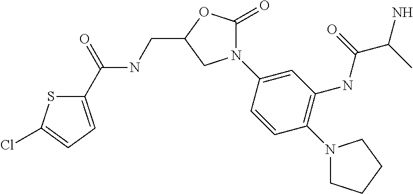 [SMILES: CC(N)C(=O)Nc1cc(N2CC(CNC(=O)c3ccc(Cl)s3)OC2=O)ccc1N1CCCC1] | 2.49 | 33.7 |
| 179 | 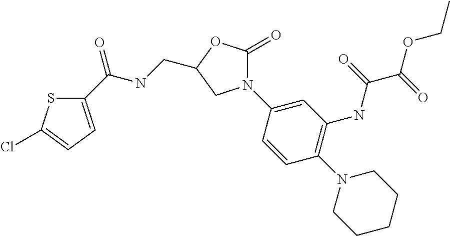 [SMILES: CCOC(=O)C(=O)Nc1cc(N2CC(CNC(=O)c3ccc(Cl)s3)OC2=O)ccc1N1CCCCC1] | 4.63 | 46.7 |
| 180 | 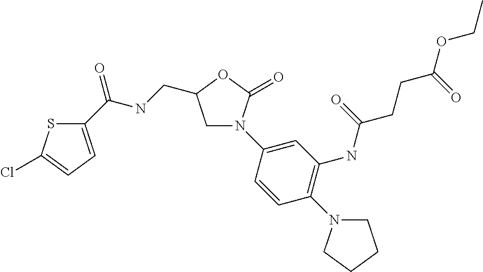 [SMILES: CCOC(=O)CCC(=O)Nc1cc(N2CC(CNC(=O)c3ccc(Cl)s3)OC2=O)ccc1N1CCCC1] | 3.37 | 44.8 |
| 181 | 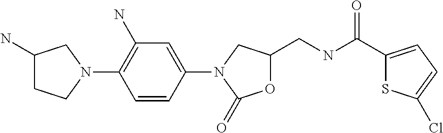 [SMILES: Nc1cc(N2CC(CNC(=O)c3ccc(Cl)s3)OC2=O)ccc1N1CCC(N)C1] | 2.16 | 83 |
| 182 | 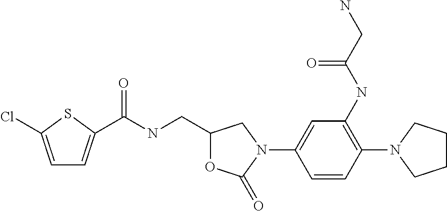 [SMILES: NCC(=O)Nc1cc(N2CC(CNC(=O)c3ccc(Cl)s3)OC2=O)ccc1N1CCCC1] | 2.31 | 93.3 |
| 183 | 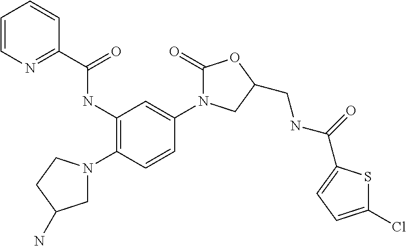 [SMILES: NC1CCN(c2ccc(N3CC(CNC(=O)c4ccc(Cl)s4)OC3=O)cc2NC(=O)c2ccccn2)C1] | 2.7 | 100 |
| 184 | 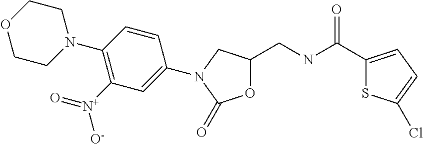 [SMILES: O=C(NCC1CN(c2ccc(N3CCOCC3)c([N+](=O)[O-])c2)C(=O)O1)c1ccc(Cl)s1] | 3.91 | 51 |
| 185 | 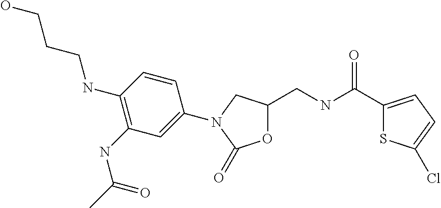 [SMILES: CC(=O)Nc1cc(N2CC(CNC(=O)c3ccc(Cl)s3)OC2=O)ccc1NCCCO] | 2.72 | 75.2 |
| 186 | 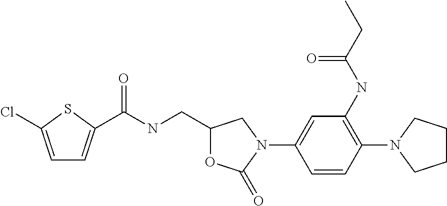 [SMILES: CCC(=O)Nc1cc(N2CC(CNC(=O)c3ccc(Cl)s3)OC2=O)ccc1N1CCCC1] | 3.17 | 46 |
| 187 | 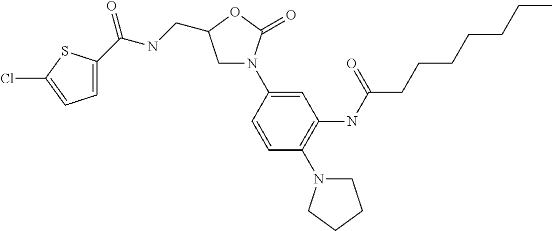 [SMILES: CCCCCCCC(=O)Nc1cc(N2CC(CNC(=O)c3ccc(Cl)s3)OC2=O)ccc1N1CCCC1] | 4.61 | 50.2 |
| 188 | 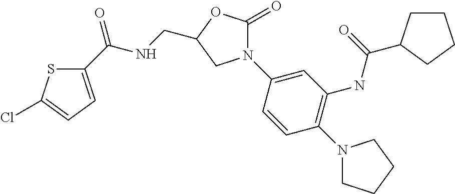 [SMILES: O=C(NCC1CN(c2ccc(N3CCCC3)c(NC(=O)C3CCCC3)c2)C(=O)O1)c1ccc(Cl)s1] | 3.89 | 56.6 |
| 189 | 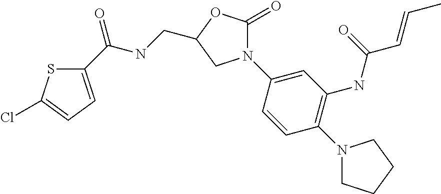 [SMILES: C/C=C/C(=O)Nc1cc(N2CC(CNC(=O)c3ccc(Cl)s3)OC2=O)ccc1N1CCCC1] | 3.37 | 52.9 |
| 190 | 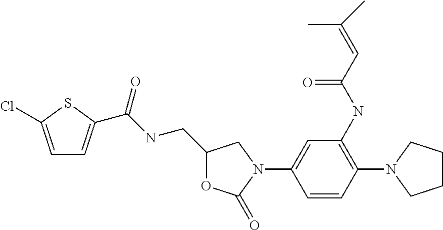 [SMILES: CC(C)=CC(=O)Nc1cc(N2CC(CNC(=O)c3ccc(Cl)s3)OC2=O)ccc1N1CCCC1] | 3.6 | 63.9 |
| 191 | 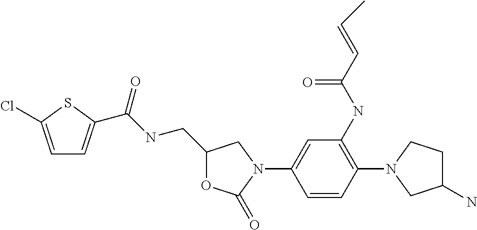 [SMILES: C/C=C/C(=O)Nc1cc(N2CC(CNC(=O)c3ccc(Cl)s3)OC2=O)ccc1N1CCC(N)C1] | 2.52 | 70.1 |
| 192 | 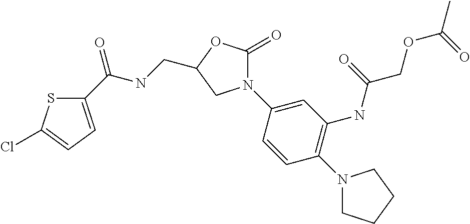 [SMILES: CC(=O)OCC(=O)Nc1cc(N2CC(CNC(=O)c3ccc(Cl)s3)OC2=O)ccc1N1CCCC1] | 3.52 | 46.6 |
| 193 | 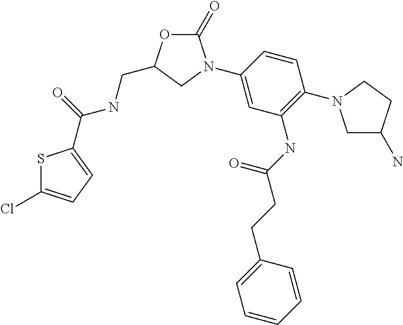 [SMILES: NC1CCN(c2ccc(N3CC(CNC(=O)c4ccc(Cl)s4)OC3=O)cc2NC(=O)CCc2ccccc2)C1] | 2.87 | 50.1 |
| 194 | 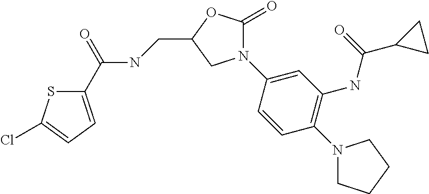 [SMILES: O=C(NCC1CN(c2ccc(N3CCCC3)c(NC(=O)C3CC3)c2)C(=O)O1)c1ccc(Cl)s1] | 3.25 | 71.1 |
| 195 | 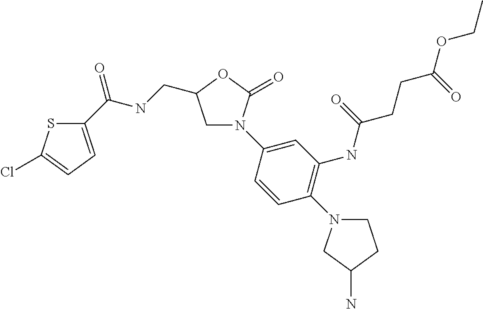 [SMILES: CCOC(=O)CCC(=O)Nc1cc(N2CC(CNC(=O)c3ccc(Cl)s3)OC2=O)ccc1N1CCC(N)C1] | 2.66 | 67 |
| 196 | 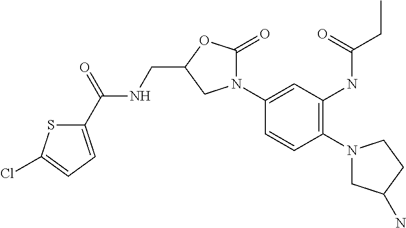 [SMILES: CCC(=O)Nc1cc(N2CC(CNC(=O)c3ccc(Cl)s3)OC2=O)ccc1N1CCC(N)C1] | 2.4 | 52.1 |
| 197 | 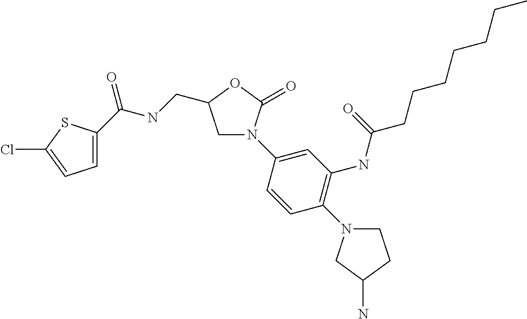 [SMILES: CCCCCCCC(=O)Nc1cc(N2CC(CNC(=O)c3ccc(Cl)s3)OC2=O)ccc1N1CCC(N)C1] | 3.13 | 48.9 |
| 198 | 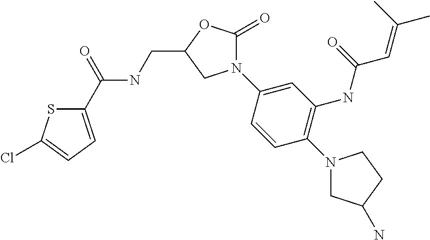 [SMILES: CC(C)=CC(=O)Nc1cc(N2CC(CNC(=O)c3ccc(Cl)s3)OC2=O)ccc1N1CCC(N)C1] | 2.67 | 75.5 |
| 199 | 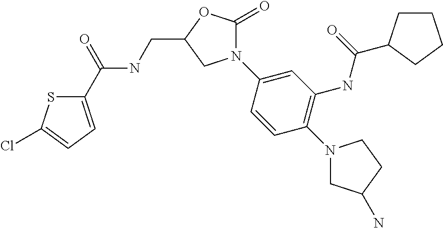 [SMILES: NC1CCN(c2ccc(N3CC(CNC(=O)c4ccc(Cl)s4)OC3=O)cc2NC(=O)C2CCCC2)C1] | 2.72 | 65.7 |
| 200 | 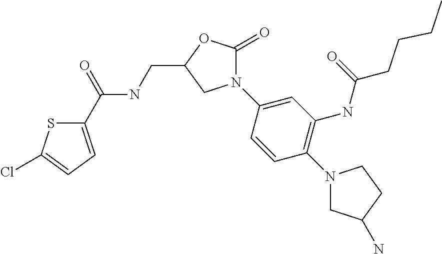 [SMILES: CCCCC(=O)Nc1cc(N2CC(CNC(=O)c3ccc(Cl)s3)OC2=O)ccc1N1CCC(N)C1] | 2.71 | 57.3 |
| 201 | 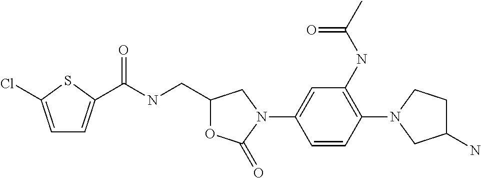 [SMILES: CC(=O)Nc1cc(N2CC(CNC(=O)c3ccc(Cl)s3)OC2=O)ccc1N1CCC(N)C1] | 2.22 | 100 |
| 202 | 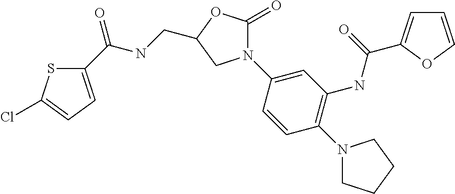 [SMILES: O=C(Nc1cc(N2CC(CNC(=O)c3ccc(Cl)s3)OC2=O)ccc1N1CCCC1)c1ccco1] | 3.89 | 75.7 |
| 203 | 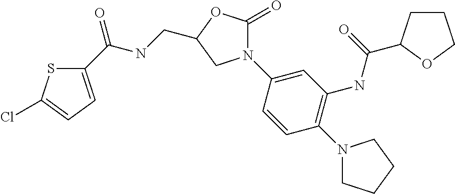 [SMILES: O=C(NCC1CN(c2ccc(N3CCCC3)c(NC(=O)C3CCCO3)c2)C(=O)O1)c1ccc(Cl)s1] | 3.19 | 49.6 |
| 204 | 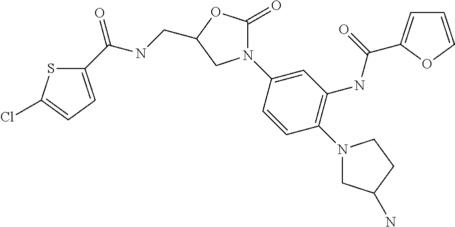 [SMILES: NC1CCN(c2ccc(N3CC(CNC(=O)c4ccc(Cl)s4)OC3=O)cc2NC(=O)c2ccco2)C1] | 2.55 | 88.2 |
| 205 | 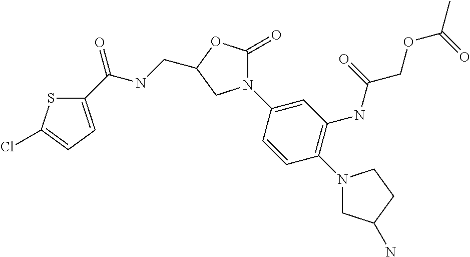 [SMILES: CC(=O)OCC(=O)Nc1cc(N2CC(CNC(=O)c3ccc(Cl)s3)OC2=O)ccc1N1CCC(N)C1] | 2.44 | 68.6 |
| 206 | 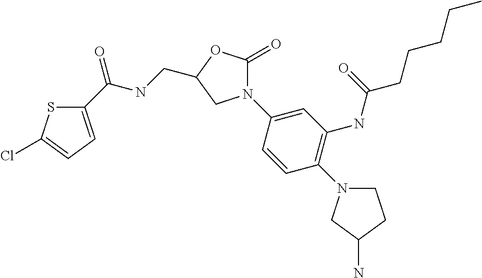 [SMILES: CCCCCC(=O)Nc1cc(N2CC(CNC(=O)c3ccc(Cl)s3)OC2=O)ccc1N1CCC(N)C1] | 2.86 | 71.8 |
| 207 | 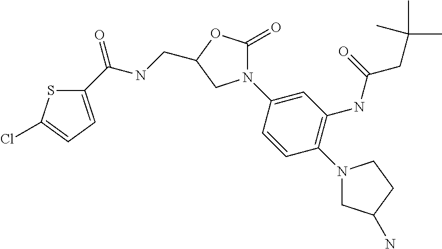 [SMILES: CC(C)(C)CC(=O)Nc1cc(N2CC(CNC(=O)c3ccc(Cl)s3)OC2=O)ccc1N1CCC(N)C1] | 2.8 | 63.6 |
| 208 | 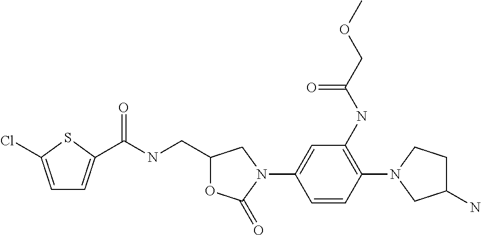 [SMILES: COCC(=O)Nc1cc(N2CC(CNC(=O)c3ccc(Cl)s3)OC2=O)ccc1N1CCC(N)C1] | 2.41 | 77 |
| 209 | 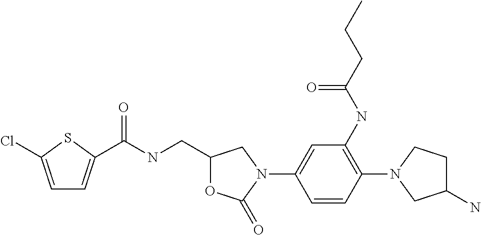 [SMILES: CCCC(=O)Nc1cc(N2CC(CNC(=O)c3ccc(Cl)s3)OC2=O)ccc1N1CCC(N)C1] | 2.56 | 67.9 |
| 210 | 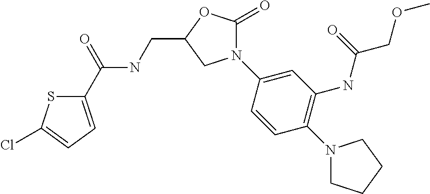 [SMILES: COCC(=O)Nc1cc(N2CC(CNC(=O)c3ccc(Cl)s3)OC2=O)ccc1N1CCCC1] | 3.67 | 78.4 |
| 211 | 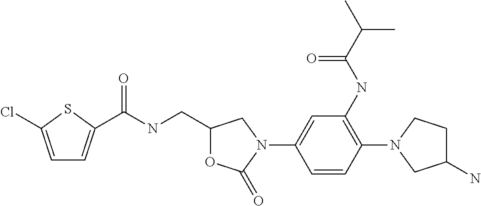 [SMILES: CC(C)C(=O)Nc1cc(N2CC(CNC(=O)c3ccc(Cl)s3)OC2=O)ccc1N1CCC(N)C1] | 2.54 | 69.8 |
| 212 | 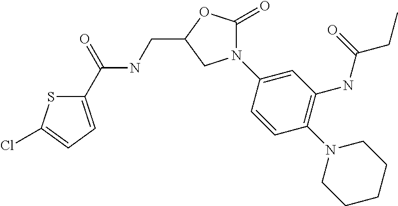 [SMILES: CCC(=O)Nc1cc(N2CC(CNC(=O)c3ccc(Cl)s3)OC2=O)ccc1N1CCCCC1] | 3.84 | 59.2 |
| 213 | 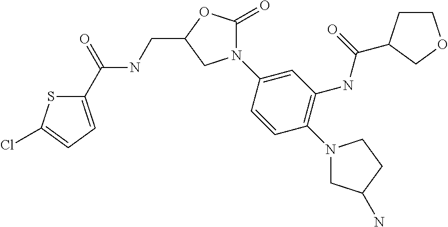 [SMILES: NC1CCN(c2ccc(N3CC(CNC(=O)c4ccc(Cl)s4)OC3=O)cc2NC(=O)C2CCOC2)C1] | 2.41 | 67.8 |
| 214 | 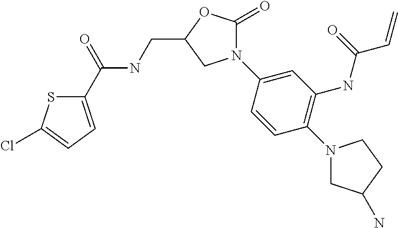 [SMILES: C=CC(=O)Nc1cc(N2CC(CNC(=O)c3ccc(Cl)s3)OC2=O)ccc1N1CCC(N)C1] | 2.41 | 75.4 |
| 215 | 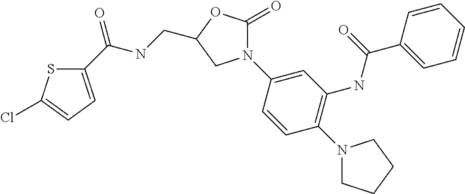 [SMILES: O=C(Nc1cc(N2CC(CNC(=O)c3ccc(Cl)s3)OC2=O)ccc1N1CCCC1)c1ccccc1] | 4.01 | 81.3 |
| 216 | 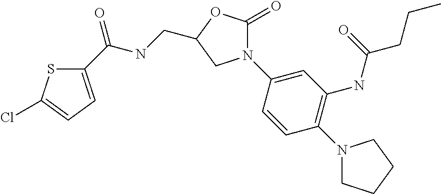 [SMILES: CCCC(=O)Nc1cc(N2CC(CNC(=O)c3ccc(Cl)s3)OC2=O)ccc1N1CCCC1] | 3.46 | 49.5 |
| 217 | 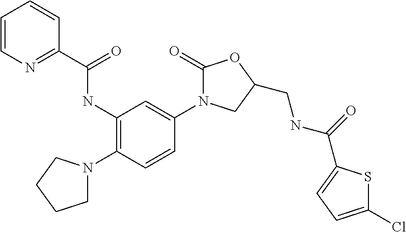 [SMILES: O=C(Nc1cc(N2CC(CNC(=O)c3ccc(Cl)s3)OC2=O)ccc1N1CCCC1)c1ccccn1] | 4.4 | 60.2 |
| 218 | 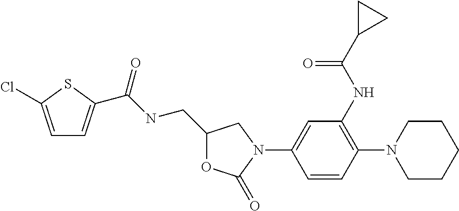 [SMILES: O=C(NCC1CN(c2ccc(N3CCCCC3)c(NC(=O)C3CC3)c2)C(=O)O1)c1ccc(Cl)s1] | 3.79 | 70.9 |
| 219 | 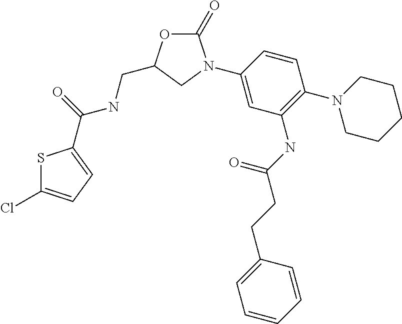 [SMILES: O=C(CCc1ccccc1)Nc1cc(N2CC(CNC(=O)c3ccc(Cl)s3)OC2=O)ccc1N1CCCCC1] | 4.57 | 51.5 |
| 220 | 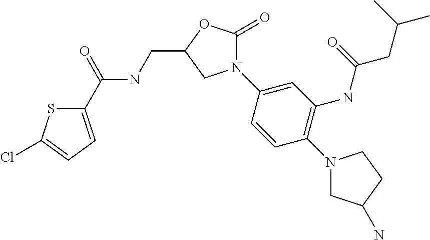 [SMILES: CC(C)CC(=O)Nc1cc(N2CC(CNC(=O)c3ccc(Cl)s3)OC2=O)ccc1N1CCC(N)C1] | 2.68 | 100 |
| 221 | 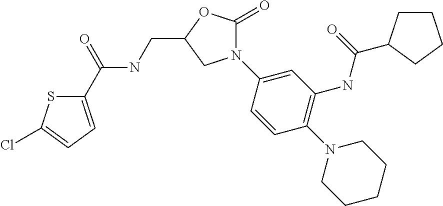 [SMILES: O=C(NCC1CN(c2ccc(N3CCCCC3)c(NC(=O)C3CCCC3)c2)C(=O)O1)c1ccc(Cl)s1] | 4.53 | 63.5 |
| 222 | 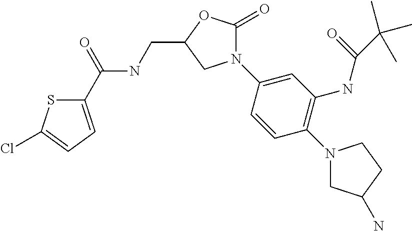 [SMILES: CC(C)(C)C(=O)Nc1cc(N2CC(CNC(=O)c3ccc(Cl)s3)OC2=O)ccc1N1CCC(N)C1] | 2.66 | 89.2 |
| 223 | 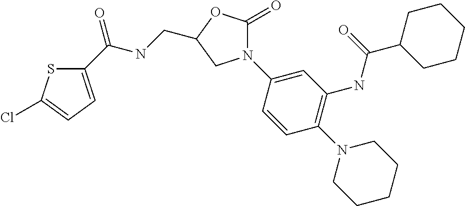 [SMILES: O=C(NCC1CN(c2ccc(N3CCCCC3)c(NC(=O)C3CCCCC3)c2)C(=O)O1)c1ccc(Cl)s1] | 4.76 | 69.3 |
| 224 | 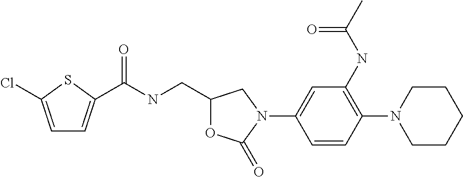 [SMILES: CC(=O)Nc1cc(N2CC(CNC(=O)c3ccc(Cl)s3)OC2=O)ccc1N1CCCCC1] | 3.45 | 77.4 |
| 225 | 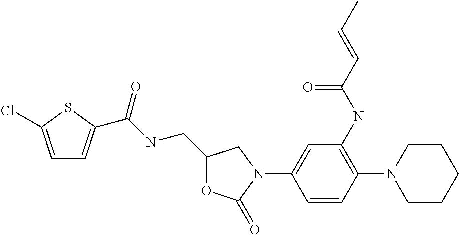 [SMILES: C/C=C/C(=O)Nc1cc(N2CC(CNC(=O)c3ccc(Cl)s3)OC2=O)ccc1N1CCCCC1] | 3.97 | 63.2 |
| 226 | 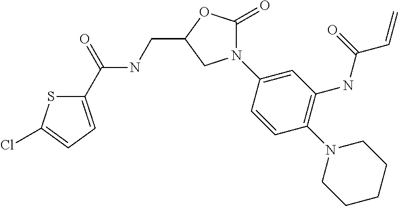 [SMILES: C=CC(=O)Nc1cc(N2CC(CNC(=O)c3ccc(Cl)s3)OC2=O)ccc1N1CCCCC1] | 3.94 | 61.4 |
| 227 | 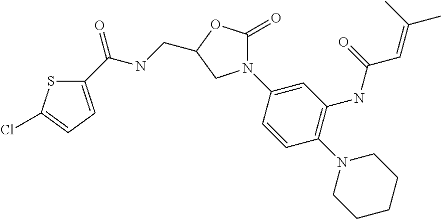 [SMILES: CC(C)=CC(=O)Nc1cc(N2CC(CNC(=O)c3ccc(Cl)s3)OC2=O)ccc1N1CCCCC1] | 4.15 | 66.3 |
| 228 | 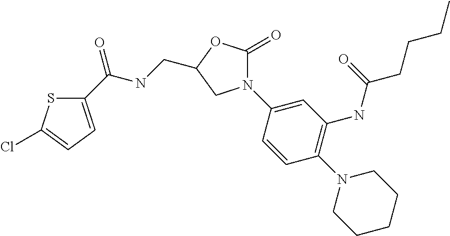 [SMILES: CCCCC(=O)Nc1cc(N2CC(CNC(=O)c3ccc(Cl)s3)OC2=O)ccc1N1CCCCC1] | 4.41 | 55.1 |
| 229 | 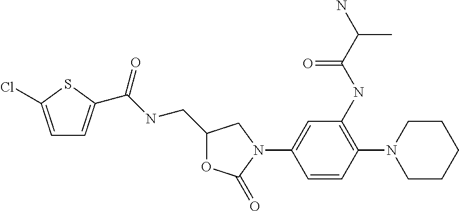 [SMILES: CC(N)C(=O)Nc1cc(N2CC(CNC(=O)c3ccc(Cl)s3)OC2=O)ccc1N1CCCCC1] | 2.83 | 41.1 |
| 230 | 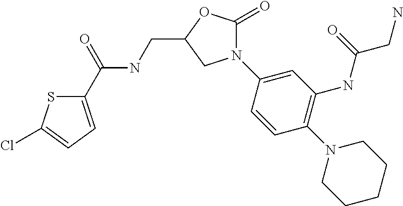 [SMILES: NCC(=O)Nc1cc(N2CC(CNC(=O)c3ccc(Cl)s3)OC2=O)ccc1N1CCCCC1] | 2.7 | 83 |
| 231 | 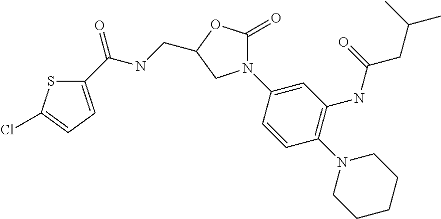 [SMILES: CC(C)CC(=O)Nc1cc(N2CC(CNC(=O)c3ccc(Cl)s3)OC2=O)ccc1N1CCCCC1] | 4.39 | 64.2 |
| 232 | 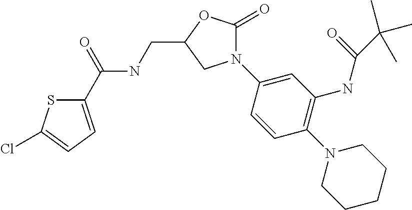 [SMILES: CC(C)(C)C(=O)Nc1cc(N2CC(CNC(=O)c3ccc(Cl)s3)OC2=O)ccc1N1CCCCC1] | 4.85 | 74.9 |
| 233 | 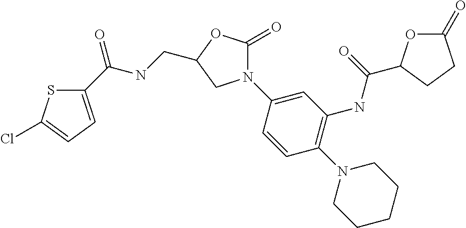 [SMILES: O=C1CCC(C(=O)Nc2cc(N3CC(CNC(=O)c4ccc(Cl)s4)OC3=O)ccc2N2CCCCC2)O1] | 4.17 | 41 |
| 234 | 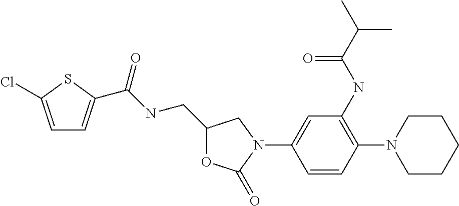 [SMILES: CC(C)C(=O)Nc1cc(N2CC(CNC(=O)c3ccc(Cl)s3)OC2=O)ccc1N1CCCCC1] | 4.21 | 61.8 |
| 235 | 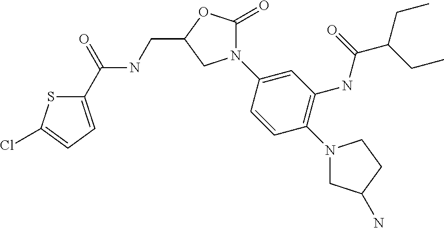 [SMILES: CCC(CC)C(=O)Nc1cc(N2CC(CNC(=O)c3ccc(Cl)s3)OC2=O)ccc1N1CCC(N)C1] | 2.75 | 100 |
| 236 | 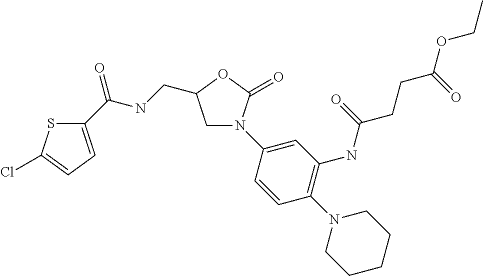 [SMILES: CCOC(=O)CCC(=O)Nc1cc(N2CC(CNC(=O)c3ccc(Cl)s3)OC2=O)ccc1N1CCCCC1] | 3.94 | 50 |
| 237 | 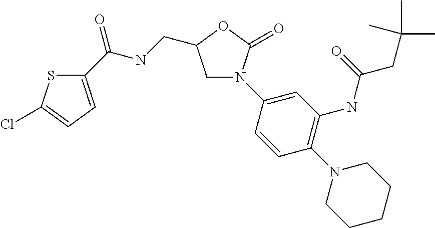 [SMILES: CC(C)(C)CC(=O)Nc1cc(N2CC(CNC(=O)c3ccc(Cl)s3)OC2=O)ccc1N1CCCCC1] | 4.65 | 75.8 |
| 238 | 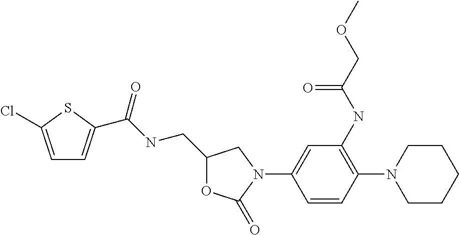 [SMILES: COCC(=O)Nc1cc(N2CC(CNC(=O)c3ccc(Cl)s3)OC2=O)ccc1N1CCCCC1] | 4.4 | 75.3 |
| 239 | 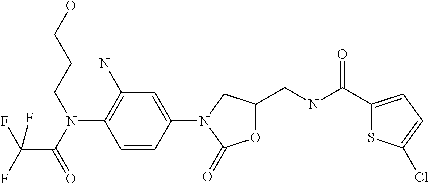 [SMILES: Nc1cc(N2CC(CNC(=O)c3ccc(Cl)s3)OC2=O)ccc1N(CCCO)C(=O)C(F)(F)F] | 4.24 | 62.2 |
| 240 | 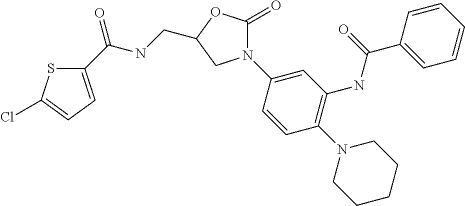 [SMILES: O=C(Nc1cc(N2CC(CNC(=O)c3ccc(Cl)s3)OC2=O)ccc1N1CCCCC1)c1ccccc1] | 4.76 | 75.1 |
| 241 | 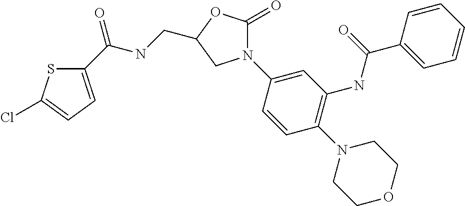 [SMILES: O=C(Nc1cc(N2CC(CNC(=O)c3ccc(Cl)s3)OC2=O)ccc1N1CCOCC1)c1ccccc1] | 4.17 | 72.5 |
| 242 | 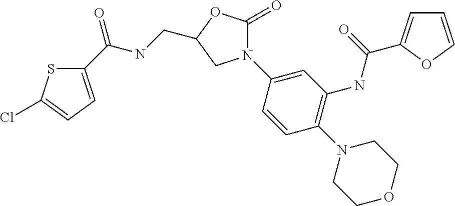 [SMILES: O=C(Nc1cc(N2CC(CNC(=O)c3ccc(Cl)s3)OC2=O)ccc1N1CCOCC1)c1ccco1] | 4.6 | 74.8 |
| 243 | 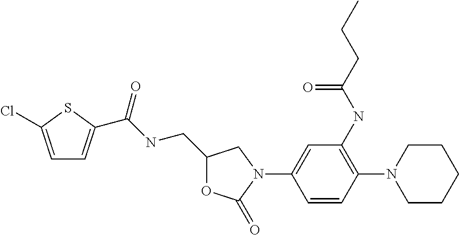 [SMILES: CCCC(=O)Nc1cc(N2CC(CNC(=O)c3ccc(Cl)s3)OC2=O)ccc1N1CCCCC1] | 4.12 | 51.6 |
| 244 | 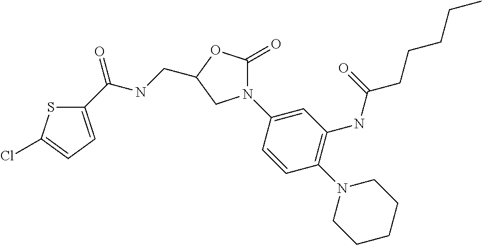 [SMILES: CCCCCC(=O)Nc1cc(N2CC(CNC(=O)c3ccc(Cl)s3)OC2=O)ccc1N1CCCCC1] | 4.71 | 66.2 |
| 245 | 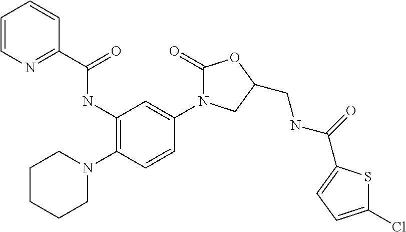 [SMILES: O=C(Nc1cc(N2CC(CNC(=O)c3ccc(Cl)s3)OC2=O)ccc1N1CCCCC1)c1ccccn1] | 4.86 | 62 |
| 246 | 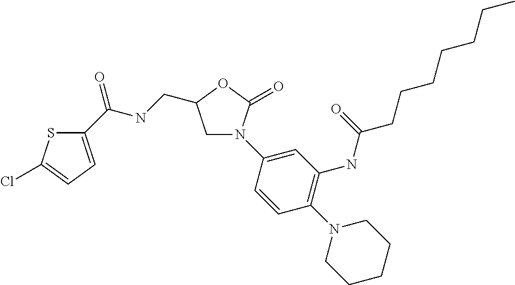 [SMILES: CCCCCCCC(=O)Nc1cc(N2CC(CNC(=O)c3ccc(Cl)s3)OC2=O)ccc1N1CCCCC1] | 5.23 | 58.3 |
| 247 | 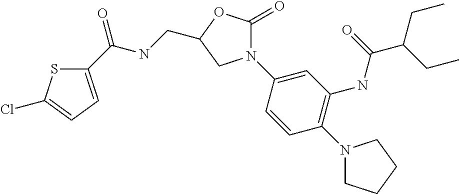 [SMILES: CCC(CC)C(=O)Nc1cc(N2CC(CNC(=O)c3ccc(Cl)s3)OC2=O)ccc1N1CCCC1] | 4.17 | 72.4 |
| 248 | 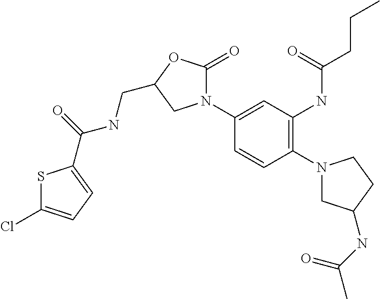 [SMILES: CCCC(=O)Nc1cc(N2CC(CNC(=O)c3ccc(Cl)s3)OC2=O)ccc1N1CCC(NC(C)=O)C1] | 3.35 | 59.6 |
| 249 | 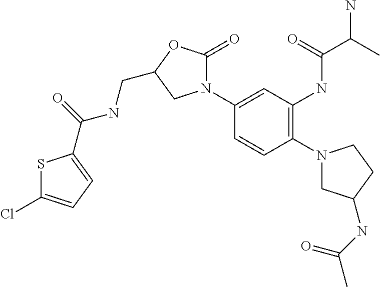 [SMILES: CC(=O)NC1CCN(c2ccc(N3CC(CNC(=O)c4ccc(Cl)s4)OC3=O)cc2NC(=O)C(C)N)C1] | 2.41 | 60.3 |
| 250 | 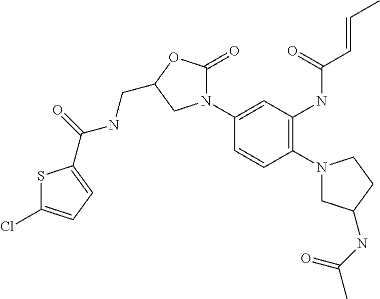 [SMILES: C/C=C/C(=O)Nc1cc(N2CC(CNC(=O)c3ccc(Cl)s3)OC2=O)ccc1N1CCC(NC(C)=O)C1] | 3.31 | 65.2 |
| 251 | 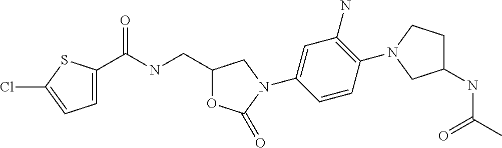 [SMILES: CC(=O)NC1CCN(c2ccc(N3CC(CNC(=O)c4ccc(Cl)s4)OC3=O)cc2N)C1] | 2.86 | 36.5 |
| 252 | 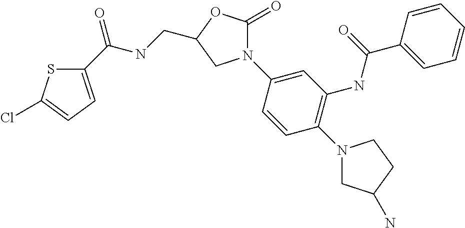 [SMILES: NC1CCN(c2ccc(N3CC(CNC(=O)c4ccc(Cl)s4)OC3=O)cc2NC(=O)c2ccccc2)C1] | 2.69 | 89.8 |
| 253 | 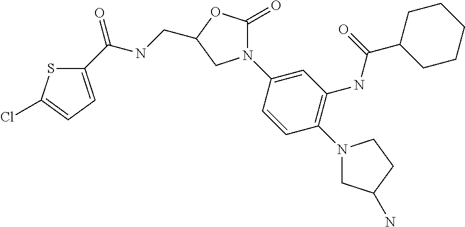 [SMILES: NC1CCN(c2ccc(N3CC(CNC(=O)c4ccc(Cl)s4)OC3=O)cc2NC(=O)C2CCCCC2)C1] | 2.81 | 67.4 |
| 254 | 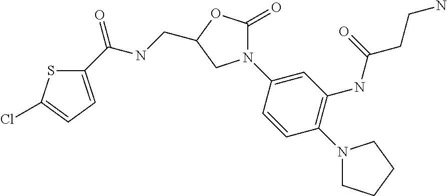 [SMILES: NCCC(=O)Nc1cc(N2CC(CNC(=O)c3ccc(Cl)s3)OC2=O)ccc1N1CCCC1] | 2.19 | 75.4 |

All products of the solid phase-assisted synthesis were characterized by LC-MS. The following separation system was routinely used for this: HP 1100 with UV detector (208-400 nm), 40° C. oven temperature, Waters Symmetry C18 column (50 mm×2.1 mm, 3.5 μm), mobile phase A: 99.9% acetonitrile/0.1% formic acid, mobile phase B: 99.9% water/0.1% formic acid; gradient:

| Time | A: % | B: % | Flow rate |
|---|---|---|---|
| 0.00 | 10.0 | 90.0 | 0.50 |
| 4.00 | 90.0 | 10.0 | 0.50 |
| 6.00 | 90.0 | 10.0 | 0.50 |
| 6.10 | 10.0 | 90.0 | 1.00 |
| 7.50 | 10.0 | 90.0 | 0.50 |

The substances were detected by means of a Micromass Quattro LCZ MS, ionization: ESI positive/negative.

The radical(s)

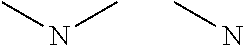

or —O present in the structures detailed above always mean a

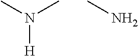

or —OH function.

1-11. (canceled)

12. A method for the prophylaxis and/or treatment of thromboembolic disorders in a patient in need thereof consisting essentially of administering a therapeutically effective amount of:
A) 5-chloro-N-({(5S)-2-oxo-3-[4-(3-oxo-4-morpholinyl)phenyl]-1,3-oxazolidin-5-yl}methyl)-2-thiophenecarboxamide of the formula 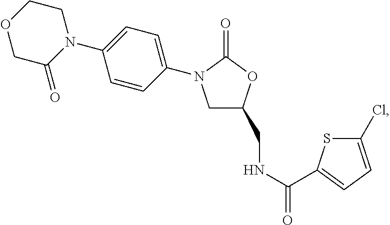 [SMILES: O=C(NC[C@H]1CN(c2ccc(N3CCOCC3=O)cc2)C(=O)O1)c1ccc(Cl)s1]
B) acetylsalicylic acid
and
C) clopidogrel
to the patient.

13. The method of claim 12, wherein the thromboembolic disorder is a myocardial infarction with ST segment elevation (STEMI) and without ST segment elevation (non-STEMI), stable angina pectoris, unstable angina pectoris, reocclusions and restenoses following coronary interventions, peripheral arterial occlusive diseases, pulmonary embolisms, deep vein thromboses and renal vein thromboses, transient ischemic attacks, or thrombotic and thromboembolic stroke.

14. The method of claim 12, wherein 5-chloro-N-({(5S)-2-oxo-3-[4-(3-oxo-4-morpholinyl)phenyl]-1,3-oxazolidin-5-yl}methyl)-2-thiophenecarboxamide of the formula 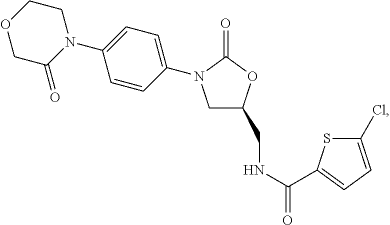 [SMILES: O=C(NC[C@H]1CN(c2ccc(N3CCOCC3=O)cc2)C(=O)O1)c1ccc(Cl)s1]
acetylsalicylic acid and clopidogrel are administered sequentially for the prophylaxis and/or treatment of the same thromboembolic disorder.

15. The method of claim 13, wherein 5-chloro-N-({(5S)-2-oxo-3-[4-(3-oxo-4-morpholinyl)phenyl]-1,3-oxazolidin-5-yl}methyl)-2-thiophenecarboxamide of the formula 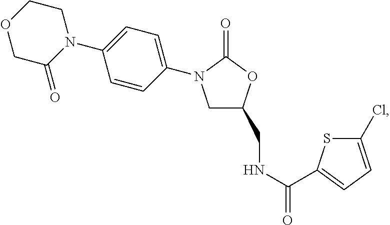 [SMILES: O=C(NC[C@H]1CN(c2ccc(N3CCOCC3=O)cc2)C(=O)O1)c1ccc(Cl)s1]
acetylsalicylic acid and clopidogrel are administered sequentially for the prophylaxis and/or treatment of the same thromboembolic disorder.

16. The method of claim 12, wherein the thromboembolic disorder is a myocardial infarction with ST segment elevation (STEMI) or without ST segment elevation (non-STEMI).

17. The method of claim 16, wherein 5-chloro-N-({(5S)-2-oxo-3-[4-(3-oxo-4-morpholinyl)phenyl]-1,3-oxazolidin-5-yl}methyl)-2-thiophenecarboxamide of the formula 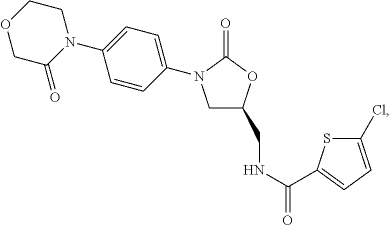 [SMILES: O=C(NC[C@H]1CN(c2ccc(N3CCOCC3=O)cc2)C(=O)O1)c1ccc(Cl)s1]
acetylsalicylic acid and clopidogrel are administered sequentially for the prophylaxis and/or treatment of the same thromboembolic disorder.

18. A method for the prophylaxis and/or treatment of thromboembolic disorders in a patient in need thereof consisting of administering a therapeutically effective amount of:
A) 5-chloro-N-({(5S)-2-oxo-3-[4-(3-oxo-4-morpholinyl)phenyl]-1,3-oxazolidin-5-yl}methyl)-2-thiophenecarboxamide of the formula 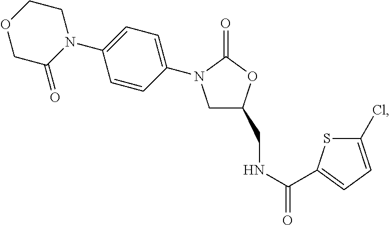 [SMILES: O=C(NC[C@H]1CN(c2ccc(N3CCOCC3=O)cc2)C(=O)O1)c1ccc(Cl)s1]
B) acetylsalicylic acid
and
C) clopidogrel
to the patient.

19. The method of claim 18, wherein the thromboembolic disorder is a myocardial infarction with ST segment elevation (STEMI) and without ST segment elevation (non-STEMI), stable angina pectoris, unstable angina pectoris, reocclusions and restenoses following coronary interventions, peripheral arterial occlusive diseases, pulmonary embolisms, deep vein thromboses and renal vein thromboses, transient ischemic attacks, or thrombotic and thromboembolic stroke.

20. The method of claim 18, wherein 5-chloro-N-({(5S)-2-oxo-3-[4-(3-oxo-4-morpholinyl)phenyl]-1,3-oxazolidin-5-yl}methyl)-2-thiophenecarboxamide of the formula 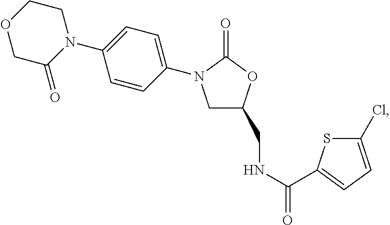 [SMILES: O=C(NC[C@H]1CN(c2ccc(N3CCOCC3=O)cc2)C(=O)O1)c1ccc(Cl)s1]
acetylsalicylic acid and clopidogrel are administered sequentially for the prophylaxis and/or treatment of the same thromboembolic disorder.

21. The method of claim 19, wherein 5-chloro-N-({(5S)-2-oxo-3-[4-(3-oxo-4-morpholinyl)phenyl]-1,3-oxazolidin-5-yl}methyl)-2-thiophenecarboxamide of the formula 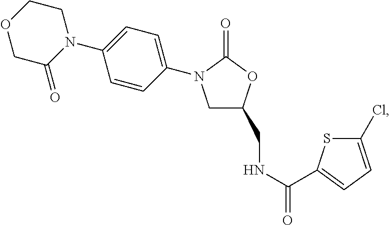 [SMILES: O=C(NC[C@H]1CN(c2ccc(N3CCOCC3=O)cc2)C(=O)O1)c1ccc(Cl)s1]
acetylsalicylic acid and clopidogrel are administered sequentially for the prophylaxis and/or treatment of the same thromboembolic disorder.

22. The method of claim 18, wherein the thromboembolic disorder is a myocardial infarction with ST segment elevation (STEMI) or without ST segment elevation (non-STEMI).

23. The method of claim 22, wherein 5-chloro-N-({(5S)-2-oxo-3-[4-(3-oxo-4-morpholinyl)phenyl]-1,3-oxazolidin-5-yl}methyl)-2-thiophenecarboxamide of the formula 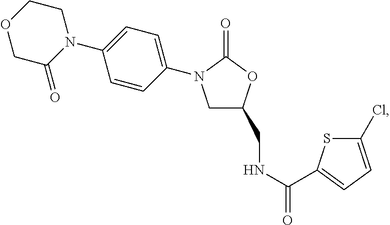 [SMILES: O=C(NC[C@H]1CN(c2ccc(N3CCOCC3=O)cc2)C(=O)O1)c1ccc(Cl)s1]
acetylsalicylic acid and clopidogrel are administered sequentially for the prophylaxis and/or treatment of the same thromboembolic disorder.
